(19) United States
(12) Patent Application Publication
(10) Pub. No.: US 2025/0281327 A1
(43) Pub. Date: Sep. 11, 2025

(54) CATARACT FRAGMENTATION AND REMOVAL DEVICES AND METHODS

(71) Applicant: THE JOHNS HOPKINS UNIVERSITY, Baltimore, MD (US)

(72) Inventors: Joshua De Souza, Baltimore, MD (US); Namratha Potharaj, Baltimore, MD (US); Kunal Parikh, Reynoldsburg, OH (US); George L Coles, Baltimore, MD (US); Samuel C Yiu, Baltimore, MD (US); Nicholas Calafat, Fort Worth, TX (US); Daniel Myers, Allentown, PA (US); Brittany Reed, Boston, MA (US); Krithik Srithar, Newburgh, IN (US); Kapil Mishra, Baltimore, MD (US); Nakul Shekhawat, Baltimore, MD (US)

(21) Appl. No.: 18/560,834

(22) PCT Filed: May 16, 2022
(86) PCT No.: PCT/US22/29475

§ 371 Date: Nov. 14, 2023

Related U.S. Application Data
(60) Provisional application No. 63/188,753, filed on May 14, 2021.

(51) Int. Cl. A61F 9/007; A61B 17/00; A61B 17/32; A61B 17/3205

(52) U.S. Cl. CPC A61F 9/00736; A61B 2017/00367; A61B 2017/0042; A61B 17/320016; A61B 2017/320052; A61B 2017/320064; A61B 17/32056; A61B 2217/007

(57) ABSTRACT

A low cost, handheld device, according to the present invention, enables surgeons to perform cataract surgery manually through a small incision in order to minimize surgically induced astigmatism and reduce recovery time. The present invention fragments cataracts into a number of individual fragments that can be removed manually or via irrigation through a small incision. The device has been designed for use in emerging markets, is less-invasive than current techniques, meets the cost and time constraints of high volume eye care systems, fits within the existing training and clinical paradigms, is comfortable and allows for dexterity of movement in the hands of a surgeon, and is able to fragment all grades of cataract, including mature cataracts, meeting the needs of the entire patient population.

### CROSS REFERENCE TO RELATED APPLICATIONS

This application claims the benefit of U.S. Provisional Patent Application No. 63/188,753 filed on May 14, 2021, which is incorporated by reference, herein, in its entirety.

### FIELD OF THE INVENTION

The present invention relates generally to medical devices. More particularly, the present invention relates to a cataract fragmentation and removal device.

### BACKGROUND OF THE INVENTION

Manual Small Incision Cataract Surgery (MSICS) is a popular method for treating cataracts in low and middle income regions and enables eye care centers within these low and middle income regions to efficiently provide low cost, high quality treatment. MSICS involves manual extraction of the whole lens nucleus through a 6-7.5 mm incision, can be performed in 4-9 minutes, and carries a low cost for the hospital. The MSICS procedure is also safe for mature cataracts which are more prevalent in rural, lower-income populations. However, the large incision can cause significant, surgically-induced astigmatism, which reduces the patient's overall quality of vision and requires an extended recovery period, which reduces quality of life and places an additional burden on the patient and their family.

The high degree of astigmatism induced in MSICS requires visual correction with expensive, cylindrical eyeglasses. However, the rate of adoption of the cylindrical glasses is low in MSICS patients who may have limited resources and for whom glasses may not be compatible or convenient. Moreover, the extended recovery period, due to the larger incision used in MSICS, increases the financial burden of treatment for patients and their families, perpetuating a cycle of delayed care, advanced disease progression, and worse surgical outcomes for the most vulnerable individuals.

Phacoemulsification, involving ultrasonic cataract emulsification and removal through a 3 mm incision, is the current gold-standard for cataract treatment, but is not optimal for emerging markets. It is neither time- nor cost-efficient, and requires significant capital expense, recurring costs, and maintenance. Moreover, it is not optimal for treating ultra-hard, late-stage cataracts which require increased ultrasound power that may be damaging.

There is, therefore, need in the art for a device and methods to allow manual cataract surgery to be safely performed on all types of cataracts through a small incision in a simple, time- and cost-effective manner.

### SUMMARY OF THE INVENTION

The foregoing needs are met, to a great extent, by the present invention which provides a device for cataract surgery including a handle and a cutting element having at least two loops. The cutting element is deployed to capture and fragment a cataract. An actuating mechanism is disposed within the handle. The actuating mechanism includes an actuating element disposed on a surface of the handle. The actuating mechanism is configured for deploying the cutting element. A passive element is configured to facilitate movement of the at least two loops of the cutting element during deployment of the cutting element.

In accordance with an aspect of the present invention, the device includes a tip coupled to the handle. The tip defines holes through which the at least two loops of the cutting element exit. The holes through which the at least two loops of the cutting element exit are positioned at the sides of the tip. The tip defines a center lumen. The tip is sized to be inserted through a 3 mm incision in the eye. The tip and handle have a hollow central lumen to facilitate removal and/or collection of fragments. The cutting element, passive element and tip act synergistically, resulting in the cutting element's lateral motion along the sides of the cataract toward a centerline of the cataract as the cutting element simultaneously expands toward a distal ends of the cataract. The tip further comprises guides that position the cutting elements around the cataract. The cutting element can include at least three loops. The cutting element is formed from a biocompatible metal or polymer. The actuating element can include a slider. The passive element includes a connector of varying lengths that is attached at a single point on each of the at least two loops. The device can also include an active supporting element. The active supporting element further comprises a retractable base.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1A illustrates a side view of a device for fragmentation and removal of cataracts, according to an embodiment of the present invention.

FIGS. 1B and 1C illustrate image views of an ex vivo human lens fragmentation, using the device according to the embodiment of the present invention illustrated in FIG. 1A.

FIGS. 1D-1G illustrate image views of the full cataract fragmentation and removal procedure, in a porcine eye, using the device according to the embodiment of the present invention illustrated in FIG. 1A.

FIGS. 2A-2I illustrate views of a views of a horizontal loop design, according to embodiments of the present invention.

FIGS. 3A-3G illustrate side views of a horizontal-loop device according to an embodiment of the present invention.

FIGS. 3H-3J illustrate side and image views of a general workflow for cataract capture and fragmentation according to an embodiment of the present invention.

FIGS. 4A-4K illustrate views of fan loop embodiments according to a device of the present invention.

FIG. 5A-5D illustrate views of a fan loop device for fragmentation and removal of cataracts according to an embodiment of the present invention.

FIGS. 5E and 5F illustrate image views of an ex vivo lens fragmentation, using the device according to the embodiment of the present invention illustrated in FIG. 4A.

FIGS. 5G-5J illustrate image views of the insertion of the fan loop device through retraction of the fan, using a device according to the embodiment illustrated in FIG. 4A.

FIGS. 5K-5N illustrate image views of the full cataract fragmentation and removal procedure, using the device according to the embodiment of the present invention illustrated in FIG. 1A.

FIG. 6A illustrates a side view of a side-curved tip embodiment according to the present invention. FIGS. 6B-6E illustrate image views of the side-curved tip deployed in an eye, according to an embodiment of the present invention.

FIGS. 7A-7H illustrate views of different embodiments of the tip of the device, according to an embodiment of the present invention.

FIGS. 8A-8F illustrate views of tip features, according to embodiments of the present invention and applicable to the embodiments of the invention illustrated in FIGS. 1-7.

FIGS. 9A and 9B illustrate side and sectional views of a handle according to an embodiment of the present invention.

FIGS. 10A-10F illustrate views of the cutting element, according to an embodiment of the present invention.

FIGS. 11A-11E illustrate views of the position of the cutting element as it exits a tip of the device, according to an embodiment of the present invention.

FIGS. 12A-12E illustrate views of motion of embodiments of the cutting element, according to an embodiment of the present invention.

FIGS. 13A-13D illustrate views of motion of embodiments of the cutting element, according to an embodiment of the present invention.

FIGS. 14A-14D illustrate views of a steerable tiered deployment of the cutting element, according to an embodiment of the present invention.

FIGS. 15A-15L illustrate views of the cutting element, according to an embodiment of the present invention.

FIGS. 16A-16D illustrate views of an actuating element for the cutting element, according to an embodiment of the present invention.

FIGS. 17A-17F illustrate views of a single actuation element, according to an embodiment of the present invention. FIGS. 17A and 17B illustrate views of a slider actuation element, according to an embodiment of the present invention. FIGS. 17C and 17D illustrate views of a rack and pinion slider actuation element, according to an embodiment of the present invention. FIGS. 17E and 17F illustrate views of the actuation of a two loop design configured to hug the cataract, according to an embodiment of the present invention.

FIGS. 18A-18F illustrate views of a multi-actuation element, according to an embodiment of the present invention.

FIGS. 19A-19F illustrate views of a stabilization element used in conjunction with the cutting element, according to an embodiment of the present invention.

FIGS. 20A-20G illustrate views of a stabilization element use in conjunction with the tip of the device, according to an embodiment of the present invention.

FIGS. 21A-21D illustrate views of a retractable stabilization element that skirts around the nucleus, according to an embodiment of the present invention.

FIGS. 22A-22C illustrate views of a retractable, penetrating stabilization element, according to an embodiment of the present invention.

FIGS. 23A-23H illustrate views of tips designed for removal of cataract fragments, according to an embodiment of the present invention.

FIGS. 24A-24E illustrate views of additional tip designs configured for removal of cataract fragments, according to embodiments of the present invention.

FIGS. 25A-25E illustrate views of a device designed to hug the nucleus, according to an embodiment of the present invention.

FIGS. 26A-26I illustrate additional views of a device designed to hug the nucleus, according to an embodiment of the present invention.

FIGS. 27A-27F illustrate a workflow for using the device designed to hug the nucleus, according to an embodiment of the present invention.

FIGS. 28A-28C illustrate schematic views of nucleus fragmentation that results from using the device designed to hug the nucleus using a 2 loop configuration in FIGS. 28A and 28B and a 3 loop configuration in FIG. 28C, according to an embodiment of the present invention.

FIGS. 29A and 29B illustrate views of a device with a bi-flap design, according to an embodiment of the present invention.

FIGS. 30A and 30B illustrate top-down views of the bi-flap, and various configurations of the flap design, according to an embodiment of the present invention.

FIGS. 31A-31F illustrate a workflow for using the bi-flap device, according to an embodiment of the present invention.

FIGS. 32A-32E illustrate side and top-down views of resting and deployed positions of the base acting as the guiderails, according to an embodiment of the present invention.

FIGS. 33A-33D illustrate views of various configurations of the base, according to the embodiment of the invention illustrated in FIG. 32.

FIGS. 34A-34G illustrate a workflow for using an embodiment of the present invention to create a three-piece fragmentation.

FIGS. 35A-35E illustrate views of a tip and a base, according to an embodiment of the present invention.

FIGS. 36A-36G illustrate views of a tip, a base, and a cutting element, according to an embodiment of the present invention.

FIGS. 37A and 37B illustrate a tip design for use with a single slider, according to an embodiment of the present invention.

FIG. 38 illustrates views of various implementations of the tip design of FIGS. 37A and 37B.

FIGS. 39A-39H illustrate views of the tip embodiments of FIGS. 37A and 37B.

FIGS. 40A-40I illustrate views of the tip embodiments of FIGS. 37A and 37B.

FIGS. 41A-41H illustrate manufacturing tools for manufacturing components of a device according to an embodiment of the present invention.

FIGS. 42A and 42B illustrate views of a device for fragmenting a cataract, according to an embodiment of the present invention.

FIGS. 43A-43C illustrate views of a handle, gear body, and slider, according to an embodiment of the present invention.

FIGS. 44A and 44B illustrate views of gear bodies, according to an embodiment of the present invention.

FIGS. 45A and 45B illustrate perspective views of a slider crank mechanism, according to an embodiment of the present invention.

FIGS. 46A-46C illustrate views of sliding mechanisms, according to an embodiment of the present invention.

FIGS. 47A-47D and 48A-48C illustrate perspective views of slider body, according to an embodiment of the present invention.

FIGS. 49A and 49B illustrate perspective views of another embodiment of a tip of a device, according to the present invention.

FIGS. 50A-50D illustrate an exemplary workflow, using a device according to an embodiment of the present invention.

FIGS. 51A-51D illustrate views of fragment removal instruments, according to an embodiment of the present invention.

FIGS. 52A and 52B illustrate views of an alternate embodiment of a fragment removal instrument, according to an embodiment of the present invention.

FIGS. 53A-53D illustrate views of removal instruments and tip attachments, according to an embodiment of the present invention.

FIGS. 54A-54D illustrate views of a tip and hub of a device, according to another embodiment of the present invention.

FIGS. 55A and 55B illustrate views of a tip and hub of a device, according to the embodiment of FIGS. 54A-54D.

FIGS. 56A-56E illustrate a workflow for fragmentation of a cataract, using a device according to an embodiment of FIGS. 54A-54D.

FIGS. 57A-57G illustrate a workflow for fragmentation of a cataract, according to an embodiment of the present invention.

### DETAILED DESCRIPTION OF THE PREFERRED EMBODIMENTS

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

The present invention relates to a low-cost, hand-held device that can enable surgeons to safely perform manual cataract surgery in all types of cataracts through a small incision in order to minimize surgically induced astigmatism and reduce recovery time. The hand-held device of the present invention fragments cataracts into a number of oblong pieces that can be removed manually, via the device itself, suction, a secondary instrument, irrigation through the incision, or in conjunction with a phacoemulsification probe or irrigation/aspiration. The device is less-invasive than current techniques, meets the cost and time constraints of eye care systems in emerging markets, fits within the existing cataract surgery workflow (minimizing the need for additional training or skill), is comfortable and allows for dexterity of movement in the hands of a surgeon, and is able to fragment all grades of cataract, including mature cataracts.

Several needs and goals were identified in the development of the device associated with the present invention. In performing the cataract surgery, a surgeon must be able to manually fragment and remove all grades of cataract nuclei through a ≤4 mm corneal or sclero-corneal incision and 8 mm internal incision to reduce astigmatism. Surgeons employing the MSICS technique, where the cataract nucleus is prolapsed, partially or completely, into the anterior chamber of the eye prior to removal, must be able to deploy and utilize the device to capture and fragment the nucleus in, and remove the fragments from the anterior chamber of the eye without injuring pertinent tissues (the iris, capsular bag, the angle, corneal endothelium) and without causing significant trauma to the original incision or the sclero-corneal tunnel/wound. Surgeons must be able to maintain current time efficiency of MSICS (near 5-9 minutes per procedure) and need a solution that is affordable for a high surgical volume and can be safely used on immature and mature cataracts. Additionally, surgeons need a tool that doesn't require significant ongoing maintenance or recurring costs.

To accomplish these goals and others, the technology meets at least the following characteristics: 1) it can be deployed through a small incision; 2) its elements fit within the anterior chamber during deployment and after deployment; 3) it stabilizes the cataract during the procedure; 4) it encapsulates and fragments the nucleus within the anterior chamber in a controlled and safe fashion that does not damage surrounding structures such as the iris, capsular bag, angle and corneal endothelium; 5) it captures and fragments all sizes and hardnesses of cataract into fragments of the appropriate sizes and shapes that can be removed through a small incision; 6) it facilitates removal of the cataract fragments; 7) it is re-useable; 8) it fits within the existing cataract training and surgical workflow; 9) it eliminates the need for capital equipment, maintenance, and electricity, 10) it can be formed from inexpensive, accessible materials, such as nylons or plastics. In preferred embodiments, the device would also be visible during the procedure, prevent anterior chamber shallowing, and provide efficient fragmentation and removal.

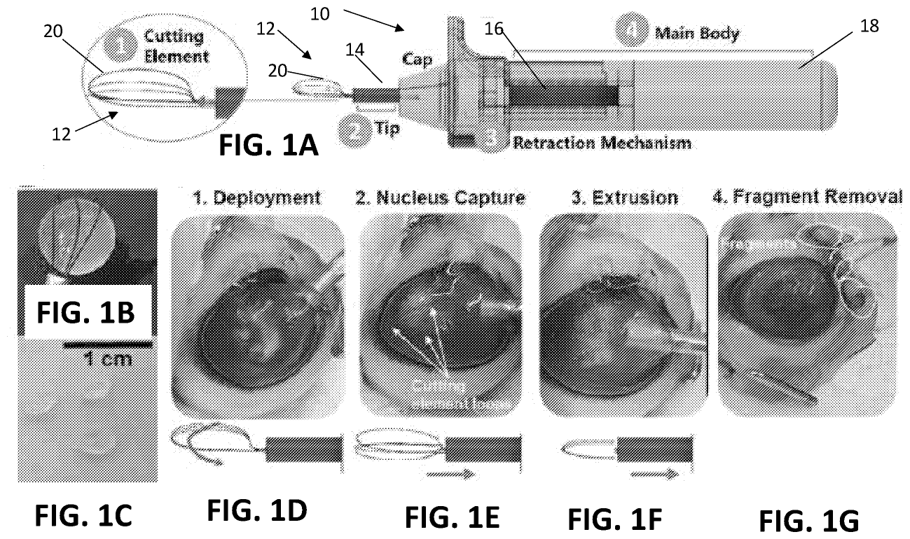

FIG. 1A illustrates a side view of a device for fragmentation and removal of cataracts, according to an embodiment of the present invention. FIG. 1A illustrates a device for fragmentation of a cataract 10, including a cutting element 12, a tip 14, and a mechanism 16 for manipulating the cutting element 12. In the embodiment illustrated in FIG. 1A, the cutting element 12 is deployed through the tip 14. The mechanism 16 for manipulating the cutting element 12 is disposed within housing 18, with an actuation element disposed on an outer surface of the housing 18, such that a physician can control the movement of the cutting element 12, enabling forward or backward translation of the cutting element and rotation of the cutting element once it is fully deployed. The cutting element 12 is deployed and then captures and fragments the cataract nucleus. The cutting element 12 can include a number of cutting loops 20 with varying configurations and geometries. As illustrated in FIG. 1A, the cutting element 12 includes three cutting loops 20. However, it should be noted that a number of different cutting element 12 implementations will be described herein, in detail, and the cutting element configuration can take any form known to or conceivable to one of skill in the art.

Further with respect to FIG. 1A, tip 14, can take the form of a tip through which the cutting element 12 is deployed into the anterior chamber (AC) of the eye. The tip 14 has a perimeter of less than approximately 8 mm, with a preferred perimeter of around approximately 6 mm. A preferred range for the perimeter of the tip is approximately 6 mm to approximately 8 mm, although smaller tip perimeters may be desirable in some embodiments and treatment circumstances. The small diameter of the tip 14 allows for a small incision, which improves patient outcomes and healing times. The tip 14 can have any geometry known to or conceivable to one of skill in the art, with various geometries including, but not limited to, rectangular, circular and elliptical shapes. The geometry of the tip can be further informed by the treatment circumstances and the particular embodiment of the present invention.

As illustrated in FIG. 1A, the mechanism 12 for manipulating the cutting element 12 is configured to extend, retract and rotate the cutting element 12 and each of its individual loops 20, in order to deploy the “flat” cutting elements partially underneath the posterior surface of the cataract to encapsulate and fragment the cataract nucleus. This fragmentation of the nucleus allows for smaller pieces of nucleus to be removed through the small incision. In a conventional MSICS, the surgeon manipulates the cataract nucleus to prolapse it from the capsular bag (a thin membrane that holds the lens) into the AC, and removes the entire nucleus through the much larger incision.

FIGS. 1B and 1C illustrate image views of an ex vivo lens fragmentation, using the device according to the embodiment of the present invention illustrated in FIG. 1A. FIGS. 1B and 1C show a successful fragmentation of a human donor lens into several small fragments. Scale is provided in FIG. 1C to show the approximate size of the fragments. Size of the fragments, of course will vary based on the overall size of the cataract and the number of fragments. Here, the cataract nucleus is shown broken into four fragments. However, it is to be understood that the number of fragments is dependent on the embodiment of the device and the treatment circumstances, including the geometry, shape, and movement of the cutting elements, and all fragmentation patterns are considered included with the present invention. Generally, it is desirable to fragment the cataract into at least two fragments. Preferably, the cataract is broken into three to four fragments. In some embodiments and under certain treatment circumstances, it may be desirable or necessary to create more than three to four fragments, as would be known to or conceivable to one of skill in the art.

FIGS. 1D-1G illustrate image views of the full cataract fragmentation and removal procedure using the device according to the embodiment of the present invention illustrated in FIG. 1A. The full procedure was performed, here, in a mature cataract pig model. In FIG. 1D, the device of the present invention, is inserted into the AC through an incision following nucleus prolapse. The incision can be sclero-corneal or corneal. The sclero-corneal incision can be internal or external, with the internal sclero-corneal incision being preferred over the corneal and the external sclero-corneal incisions. Preferably, for any of the incision types, a small incision is made in order to avoid the post-surgical complications described above. More particularly, the corneal incision has a range of approximately 2.75 mm to approximately 3.5 mm. The external sclero-corneal wound can range between approximately 3 mm to 12 mm, with a preferred incision size range of approximately 3 mm to approximately 4 mm. The internal sclero-corneal wound can range between approximately 1 mm to approximately 12 mm with a preferred incision size range of approximately 6 mm to approximately 8 mm. Further with respect to FIG. 1D, the image view of the tip, below the image of the eye, shows the device in the first stage of deployment.

In FIG. 1E, the tip of the device is inserted through the incision to capture the nucleus from below, while the loops of the cutting element are still flattened. The nucleus will be placed within the vacant center of the loop or cutting element. Once in position, the surgeon will then use the rotational mechanism to open the loops and encompass the nucleus, as shown by the image view of the tip below the image view of the eye. Once the nucleus is captured, the cutting element will be retracted, slicing the nucleus into distinct pieces, as illustrated in FIG. 1F. The image view of the tip shows retraction of the cutting element to fragment the nucleus. FIG. 1G illustrates removal of the nucleus fragments from the AC. The cutting element and the encapsulation method of the present invention are designed to minimize damage to the corneal endothelium, the iris, and capsular bag, situated above and below the device, respectively.

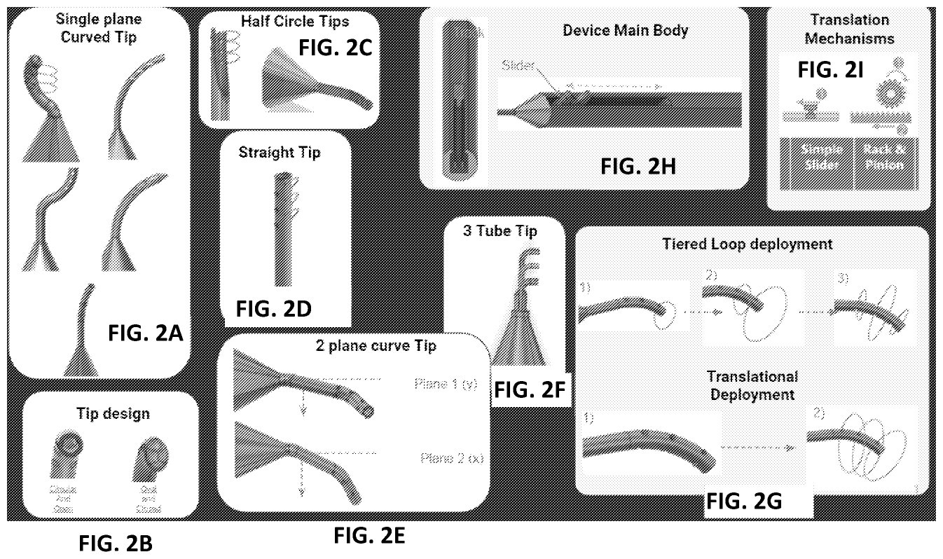

FIGS. 2A-2I illustrate views of a horizontal loop design, according to embodiments of the present invention. The tip of the device for cataract fragmentation and removal can take a number of forms. As illustrated in FIGS. 2A-2I, the tip is further structured to house the cutting element and provide direction to the deployment of the cutting element, such that when the tip is inserted into the AC, the cutting element can be deployed through holes along the surface of the tip of specific dimensions, location and angle for encapsulation and fragmentation of the cataract nucleus. The holes provide stability, directionality, and enable the cutting elements to encapsulate, capture and fragment the nucleus. The tip also acts as a supportive structure to further stabilize the nucleus during encapsulation and fragmentation, and may also directly aid fragmentation in some embodiments. The cutting elements are deployed to the side of the cataract, along the cataract “rim” and move inwards laterally.

The tip can have a number of different shapes and angles, as well as a number of different exit patterns, as defined by the trajectory of the hole placement, shape, size and number on the surface of the tip from which the cutting elements exit from and enter through. It should be noted that the tip examples for the horizontal loop design shown in FIGS. 2A-2G are not meant to be considered limiting or encompass the entire breadth of tip designs possible for the present invention. Indeed, additional embodiments of the tip are described herein. It is also possible that a kit according to the present invention could be packaged with more than one tip to allow the physician to select the most appropriate configuration for the patient's particular procedure.

FIG. 2A illustrates a single plane curved tip design with an exit pattern for three cutting loops. FIG. 2B illustrates end views of the tip. The first end view shows a tip having a circular cross-sectional shape and defining an open center lumen. The second end view shows an oval shape and closed center lumen that facilitates a smoother entry into through the tunnel. These elements can be interchanged and can also be applied to the different tip designs herein. FIG. 2C illustrates a tip with a half circle design, which provides additional surface area facing the side of the cataract in order to provide increased stability, and an exit pattern configured for three cutting loops. FIG. 2D illustrates a tip with a straight design and an angled exit pattern configured for three cutting loops, which provides directionality to the three cutting loops as they are deployed. FIG. 2E illustrates a two-plane curved tip with an exit pattern configured for three cutting loops. The two-plane configuration enables the tip to go slightly under the nucleus and capture it partially from the bottom. The two-plane configuration minimizes the space taken by the cutting element and tip, by utilizing the space within the AC and above the capsular bag. The two-plane configuration further keeps the cutting elements close to the surface of the cataract, and therefore, reduces the risk of damaging the corneal endothelium. FIG. 2F illustrates a three tube tip.

With respect to any implementation or design of the tip of the device of the present invention, the tip of the present invention has a small perimeter size to facilitate passage through a small corneal or a sclero-corneal incision. The tip of the present invention has a perimeter of less than approximately 8 mm, with a preferred perimeter of around approximately 6 mm. A preferred range for the perimeter of the tip is approximately 6 mm to approximately 8 mm, although smaller tip perimeters may be desirable in some embodiments and treatment circumstances. The small diameter of the tip allows for a small incision, which improves patient outcomes and healing time. The tip can have any geometry known to or conceivable to one of skill in the art, with various geometries including, but not limited to, rectangular, circular and elliptical shapes. The geometry of the tip can be further informed by the treatment circumstances and the particular embodiment of the present invention. For embodiments of the tip defining a lumen or other opening, a perimeter of that inner lumen can in a range of, at minimum, approximately 0 mm at a distal end of the tip to at maximum, approximately 5.9 mm. Preferably, the range for the inner perimeter is approximately 0.3 mm to approximately 4 mm.

FIG. 2G illustrates tiered cutting elements used in the horizontal loop embodiment. These tiered cutting elements can have a translational deployment in which the loops are deployed one after another to varying sizes over time. The sizes of the loops along the tip correspond to the size of the cross-section of the nucleus as they enter the central lumen of each cutting element. This enables the cutting element to minimize the space occupied in the AC, to be positioned flush with the surface of the cataract and prevent any obstruction to capturing the cataract. FIG. 2H illustrates the main body of the horizontal loop embodiment, along with the track, which allow smooth and linear translations of the cutting elements and prevent buckling of these wires, and slider, while FIG. 2I illustrates examples of the translation mechanism for the sliders, including a simple slider and a rack and pinion design that provides a greater degree of control, dependent on the number of teeth in the gear, while maintaining the hand and finger position of the surgeon through the whole process.

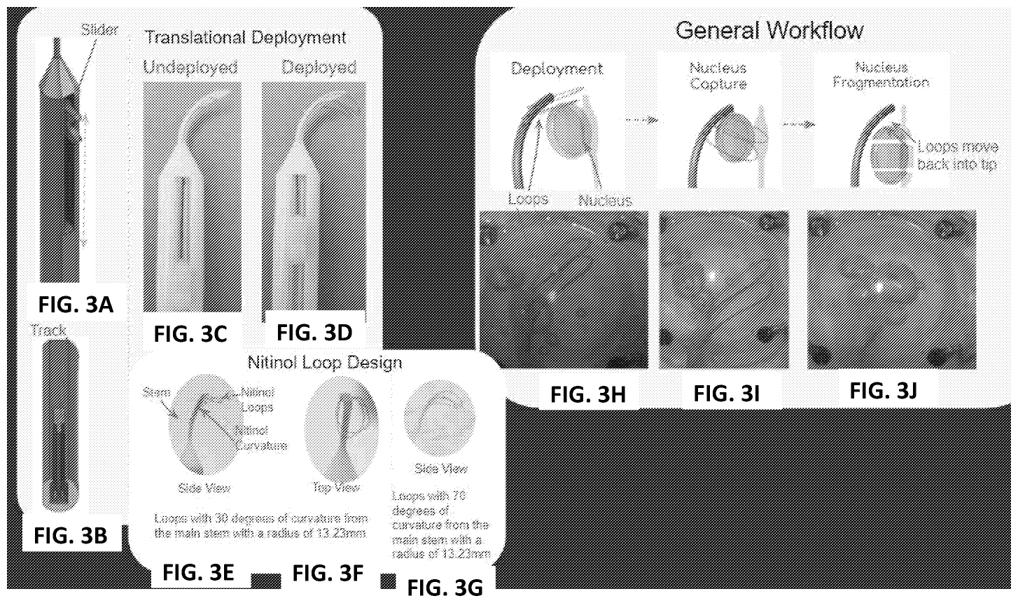

FIGS. 3A-3G illustrate views of a horizontal-loop device according to an embodiment of the present invention. FIGS. 3A-3G further illustrate a method of using the horizontal loop embodiment of FIGS. 2A-2I. FIGS. 3A and 3B illustrate the housing of the device, the slider, and the track. FIGS. 3C and 3D illustrate the tip with undeployed and deployed cutting loops, respectively. These cutting loops are also flush with the tip when stored so as to prevent snagging of tissue near the wound during insertion or removal of the device. FIGS. 3E-3G illustrate the cutting loops of the device. The designs shown in FIGS. 3A-3G show a horizontal loop design with two cutting loops. The number of cutting loops used in the design can vary and any number of cutting loops known to or conceivable to one of skill in the art can be used.

In the embodiment shown in FIGS. 3E-3G, the device includes cutting loops formed from super-elastic, round nitinol wire of approximately 0.003 inches to approximately 0.005 inches in diameter. In FIG. 3E the loops are shown with a 30-degree curvature from the main stem with a radius of 13.23 mm. In FIG. 3G the loops are shown with a 70-degree curvature from the main stem with a radius of 13.23 mm.

With respect to any of the cutting loops of the present invention, it is possible to use a number of different materials with varying diameters and cross sectional shapes. In some instances, it is possible to use super-elastic nitinol wire with varying cross-sectional diameter and geometry. In some instances, the cross-sectional diameter can further vary along the length of the wire. The diameter of the wire can be between approximately 0.003 inches to approximately 0.005 inches in diameter in preferable embodiments, and may be smaller or larger in other embodiments. The cross-sectional shape of the super-elastic nitinol wire can be round, flat or any other geometry known to or conceivable to one of skill in the art. The loops can alternately be formed from any other material known to or conceivable to one of skill in the art, including but not limited to other shape memory metals, steel, metal alloys, nylon, polymers, or other plastics or elastomers. If nylon is used, it can take the form of a nylon thread in a range between 6-0 to 2-0 suture sizes, which is approximately 0.07 mm to approximately 0.3 mm.

In any of these embodiments, the physician may utilize a sinskey hook or similar instrument to position the nucleus appropriately as the loops are expanding and capturing the nucleus. This instrument in some cases could take the form of a blunt, curved cannula with a curvature similar to that of the anterior chamber of the eye. In other cases, the side instrument can have a flat surface, or it can have a cupped configuration to allow for further stabilization of the posterior surface of the nucleus. The side instrument may be utilized along any radial position on or near the limbus of the eye either through a side port incision or paracentesis or through the main wound, per the surgeon's preference.

FIGS. 3H-3J illustrate side and image views of a general workflow for cataract capture and fragmentation according to an embodiment of the present invention. FIG. 3H illustrates deployment of the cutting loops, and positioning of the cutting elements at the distal end of the nucleus in preparation for its capture. FIG. 3I illustrates nucleus capture with the cutting loops, whereby the nucleus is guided into the loops and the loops are positioned for cutting the nucleus, and FIG. 3J illustrates nucleus fragmentation with the retraction of the cutting loops back into the tip while the cataract is stabilized by the tip.

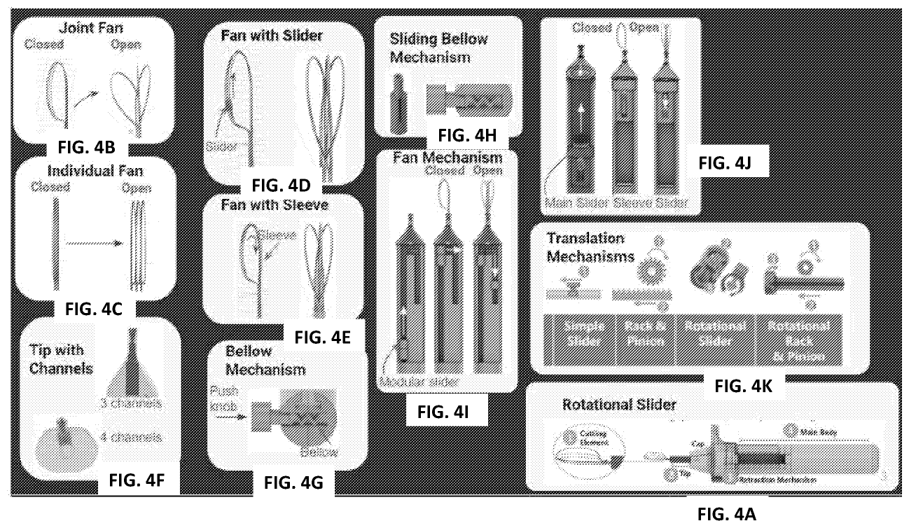

FIGS. 4A-4K illustrate views of fan loop embodiments according to a device of the present invention. FIG. 4A illustrates a perspective view of a fan loop device. FIGS. 4B-4E illustrate the loops of the fan blade in open and closed positions and with different configurations. The fan can take a joint fan configuration where the fan loops extend from one base, as illustrated in FIG. 4B. The fans can also take an individual fan configuration where the fan loops each extend from their own base, as illustrated in FIG. 4C. FIG. 4D illustrates a fan with a slider, and FIG. 4E illustrates a fan with a sleeve, either of which functions as a mechanism to control the separation and joining of the fan loops (or the opening and closing of the fan).

FIG. 4F illustrates a tip with channels through which the loops exit. Depending on the configuration used, the tip can preferably have two to four channels for the loops to exit. However, in some instances, there may only be one channel from which the loops exit. The channels can have a diameter of approximately 0.1 mm to approximately 6 mm. The channels can have any cross-sectional geometry known to or conceivable to one of skill in the art, including but not limited to circular, ellipsoid, and quadrilateral. These channels may be angled. Each fan loop may exit from its individual channel and enter through its individual channel or through a central lumen with the other fan loops. In some embodiments, spacing of these channels and the angle of the channels determines the spacing between the deployed fan loops and the angle at which they are deployed. The entry point(s) for the fan loop wire must be below the plane of the exit points for these loop wires in the instances where the wire must form an area that can capture the cataract from the side. Entry and exit points can be parallel or angled. In some cases, the tip contains a lumen through which the sleeve or slider can be retracted and deployed. All of the exit points or channels can be in the same anterior plane relative to each other and all entry points or channels (if there are multiple) can be in the same plane relative to each other.

FIGS. 4G-4K illustrate configurations for the mechanism for deploying and controlling the fan loops. The mechanism for deploying and controlling the fan loops can take the form of a bellow mechanism that is compressed in one position and expanded in another with the ends of the cutting elements being positioned at the edges of the bellow, as illustrated in FIG. 4G, or a sliding bellow mechanism, as illustrated in FIG. 4H. The bellow mechanism can include a push knob and bellow. The bellow actuates the opening and closing of the fan loops while maintaining the plane of the loops. In another embodiment of the mechanism for deploying and controlling the fan loops, illustrated in FIG. 4I, the slider can take the form of a modular slider. The slider is advanced from a proximal position to a distal position to deploy the loops. Then, the distal portion of the slider is translated through a channel across the width of the device and moved from a distal position to a proximal position to open the fan. As illustrated in FIG. 4J, the slider can alternately take the form of a two-part slider: a main slider to deploy the loops and a second slider to open and close the loops. The second slider, which is housed within the main slider, is able to move in tandem with the main slider, and able to move up and down when the main slider is stationary as shown in the figures. In some embodiments, there may be a locking mechanism for the main slider to allow for smooth retraction of the second slider once the sliders reach the end of the track, proximal to the tip. In other embodiments, there may be a rotational mechanism to rotate all of the fan loops from a flattened position (parallel to posterior surface) to a closed side position, before opening the fan through retraction of the second slider.

FIG. 4K illustrates potential embodiments for the translation mechanism. None of these specific examples of loop configurations, tip designs, sliders, or translation mechanisms should be considered limiting and are included simply by way of example. Any particular implementation known to or conceivable to one of skill in the art could also be used.

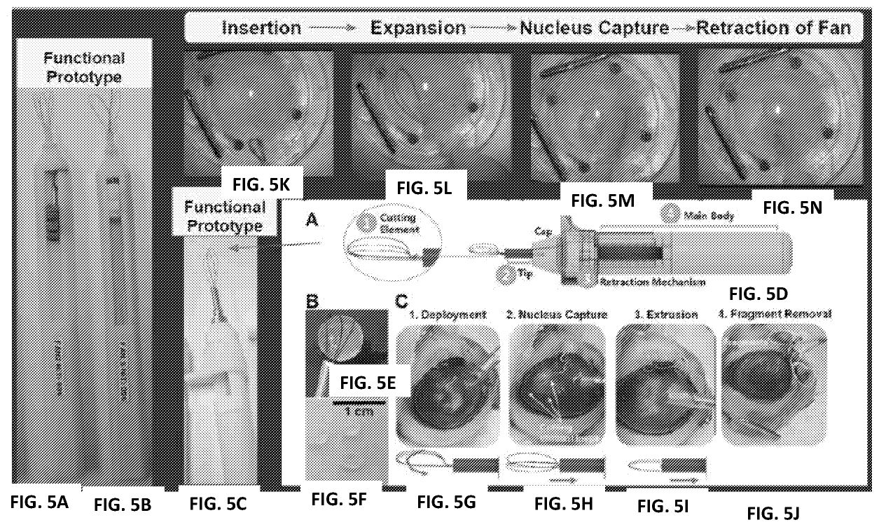

FIG. 5A-5D illustrate views of a fan loop device for fragmentation and removal of cataracts according to an embodiment of the present invention. The prototype is used in the following exemplary fragmentation and removal processes to show how it works. FIGS. 5E and 5F illustrate image views of an ex vivo lens fragmentation, using the device according to the embodiment of the present invention illustrated in FIG. 4A. FIG. 5E illustrates the fan loops extending around the cataract nucleus, and FIG. 5F illustrates the fragmented nucleus as a result of using the device.

FIGS. 5G-5J illustrate image views of the insertion of the fan loop device through retraction of the fan, using a device according to the embodiment illustrated in FIG. 5A. FIG. 5G illustrates insertion of the tip through a sclero-corneal incision and tunnel, into the AC without the deployment of the cutting elements following nucleus prolapse. The image view of the tip, below the image of the eye, shows the device in the first stage of deployment. In FIG. 5H, the loops are positioned to either side of the nucleus as the loops are deployed as a singular unit from the tip of the device. Once deployed from the tip and positioned to the side of the nucleus, the cutting elements will unfold. The nucleus will be placed within the vacant center of the loops. Once in position, the surgeon will then use the rotational mechanism to open the loops and encompass the nucleus, as shown by the image view of the tip below the image view of the eye. Once the nucleus is captured, the cutting element will be retracted, slicing the nucleus into distinct pieces, as illustrated in FIG. 5I. The image view of the tip shows retraction of the cutting element to fragment the nucleus. FIG. 5J illustrates removal of the nucleus fragments from the AC. The cutting element and the encapsulation method of the present invention are designed to minimize damage to the corneal endothelium and capsular bag, situated above and below the device, respectively.

FIGS. 5K-5N illustrate image views of the full cataract fragmentation and removal procedure using the device according to the embodiment of the present invention illustrated in FIG. 5A. FIG. 5K illustrates insertion of the device into the AC. FIG. 5L illustrates expansion of the cutting loops, FIG. 5M illustrates nucleus capture with the cutting loops, and FIG. 5N illustrates nucleus fragmentation with the retraction of the cutting loops back into the tip.

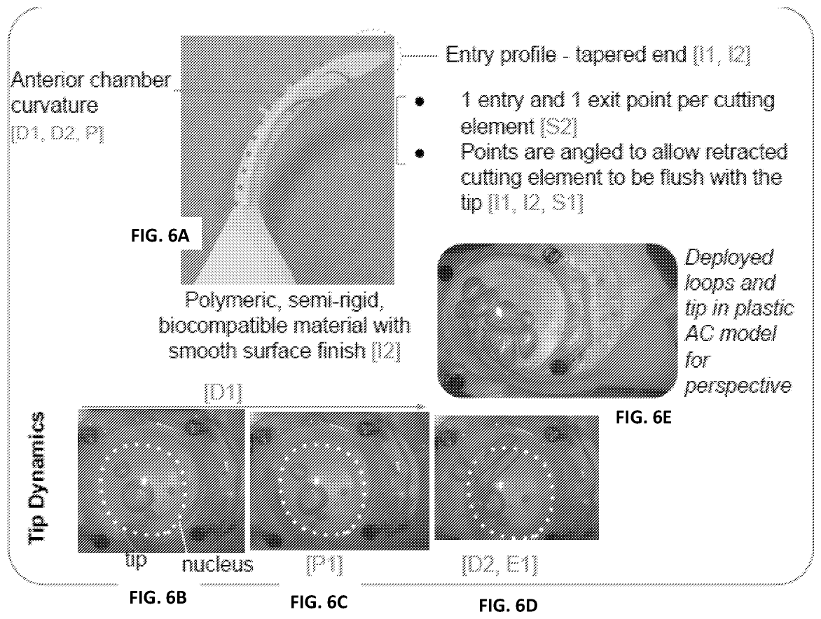

FIG. 6A illustrates a top-down view of a side-curved tip embodiment according to the present invention. FIGS. 6B-6E illustrate image views of the side-curved tip deployed in an anterior chamber model. As illustrated in FIG. 6A, the side-curved tip includes an anterior chamber curvature. The entry profile of the tip defines a tapered end for less traumatic entry. There is a single entry and exit point per cutting element and these entry and exit points are angled in order to allow the retracted cutting element to be flush with the curvature of the tip of this embodiment, as illustrated in FIG. 6A. However, this exemplary embodiment is not meant to be considered limiting, as there are a number of different loop configurations of the cutting element, and these different configurations, in turn, influence the architecture of the entry and exit points. As illustrated in FIGS. 6B-6E the tip is inserted into the wound, to the side of the nucleus and the loops, which are oriented laterally, are deployed to capture the nucleus. The scale of the device within the AC and the shape of the curve relative to the AC is also shown in these figures.

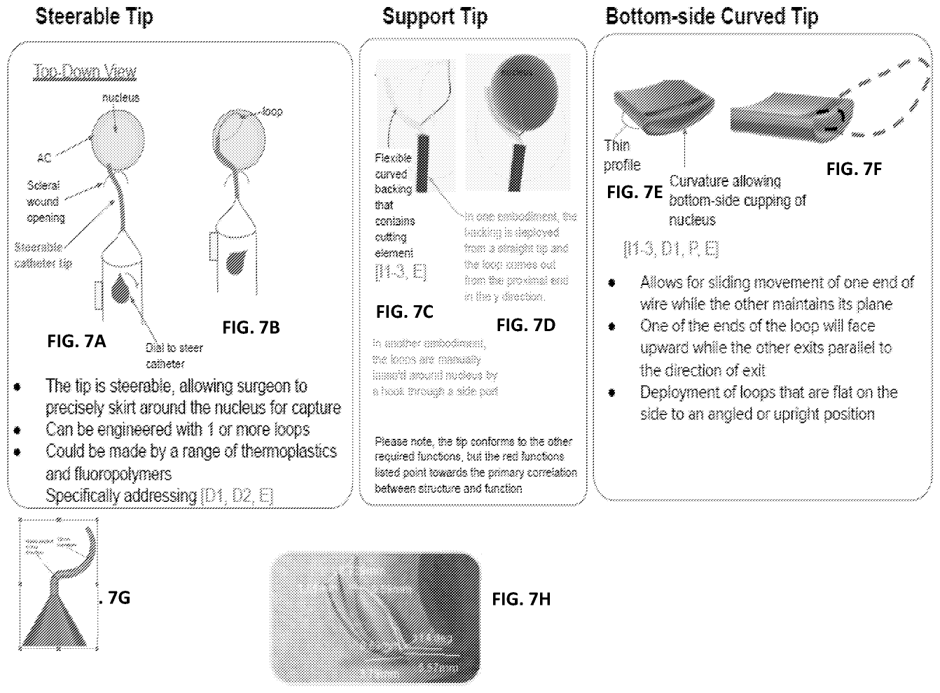

FIGS. 7A-7F illustrate views of different embodiments of the tip of the device, according to the invention. FIGS. 7A and 7B illustrate perspective views of a steerable tip. FIGS. 7C and 7D illustrate perspective views of a support tip and, FIGS. 7E and 7F illustrate perspective views of a bottom side curved tip.

The steerable tip of FIGS. 7A and 7B allows the surgeon to precisely skirt around the nucleus in multiple planes due to its maneuverability, thus enhancing nucleus capture. The steerable tip can be configured for use with any of the loop configurations described herein. The steerable tip can be formed from a wide range of thermoplastics, fluoropolymers, or other suitable biocompatible materials such as those commonly used in catheters. Any other suitable material known to or conceivable to one of skill in the art could also be used.

The support tip has a flexible curved backing that houses the cutting element or elements. In one implementation, illustrated in FIGS. 7C and 7D, the flexible curved backing is deployed from a straight tip and the loop comes out of a proximal end of the flexible curved backing in a y-direction. In another implementation, the loops are manually lassoed around a nucleus by a hook through a side port in the flexible curved backing.

The bottom-side curved tip of FIGS. 7E and 7F has a thin profile and a curvature that allows for bottom-side cupping of the nucleus of the cataract. This embodiment allows for a sliding movement of one end of the cutting element, while the other end maintains its plane. One of the ends of the cutting element will face upward, while the other exits parallel to the direction of exit. The bottom-side curved tip also allows for deployment of cutting element loops that are flat on the side to an angled or upright position.

FIGS. 7G and 7H illustrate a supporting branch, and holes that are angled away from the direction of the supporting tip. The holes that are angled away allow for curved cutting elements to be deployed around the nucleus that are counteracted/stabilized by the force applied on the nucleus by the supporting branch. As shown in FIG. 7G, the cutting elements may be deployed out of the same tip as the supporting branch, or as in FIG. 7H, the cutting elements may be deployed out of a connected but independent branch. These branches can be made of flexible biocompatible materials, known to or conceivable to one of skill in the art.

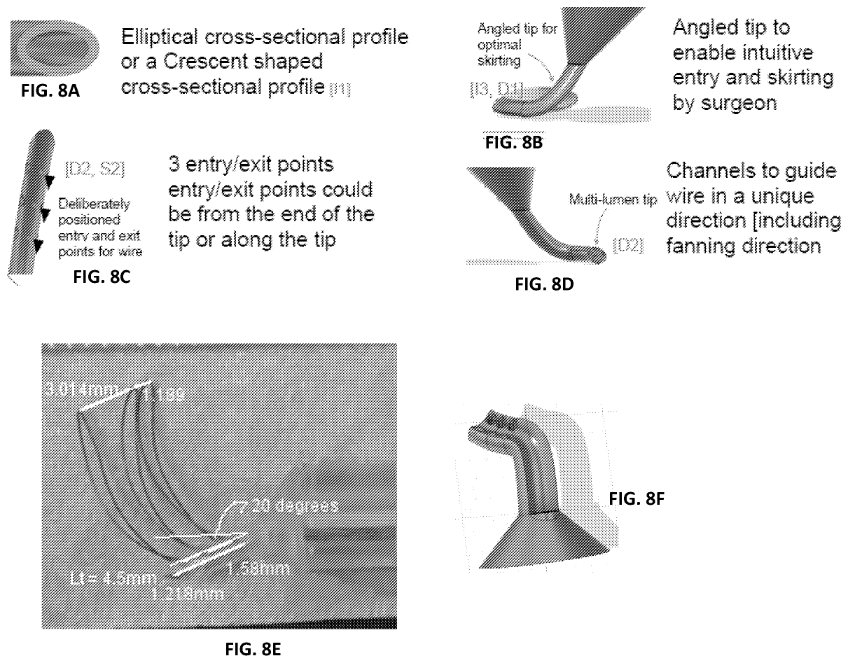

FIGS. 8A-8D illustrate additional tip features that could be used in conjunction with the embodiments of the invention illustrated in FIGS. 1-7. FIG. 8A illustrates a tip with an elliptical cross-sectional profile that mimics the shape of the incision and the maximum expandable size of the nucleus. This profile can also be more tapered on the ends to form a crescent shape. FIG. 8B illustrates an angled or curved tip to enable intuitive entry and skirting of the nucleus by the physician. FIG. 8C illustrates the entry and exit points for the loops of the cutting element. In FIG. 8C, there are three pairs of entry and exit ports that could be from the end of the tip or along a length of the tip. Finally, FIG. 8D shows channels that are configured to guide the loop in a unique direction. This can include guiding the loop in a fanning direction.

FIGS. 8E and 8F show configurations of angled tips, which allow the loops to deploy to the side of the nucleus. The cutting elements that are deployed through such tips are curved according to the curvature of the anterior chamber. In the case where both ends of the wire that is forming the loop are being translated, the curvature of the top and bottom portions of the wire are the same. In the case where one end of the wire is fixed and the other end is translated, the curvatures of the top and bottom portions of the wire may be greater than, equal to or less than each other to produce a variety of movements of the loop. Tip materials can include biocompatible metals including stainless steel, or a range of biocompatible polymeric and plastic materials including silicone, polyethylene, polypropylene, and nylon. The material used should have a stiffness in a preferred range and be biocompatible. This listing of potential materials is not meant to be considered limiting, and any biocompatible material known to or conceivable to one of skill in the art could also be used. The additional features of FIGS. 8A-8F can be implemented separately, together, or may not be applicable to some embodiments of the present invention. These additional features are also not meant to be considered limiting, and any number of additional features or variations known to or conceivable to one of skill in the art could also be implemented.

As described above, FIGS. 6A-6E; 7A-7F; and 8A-8F illustrate a tip design that is curved to the side. Other embodiments, which can be a modification or addition to the side-curved tip, or a stand-alone embodiment, include the steerable tip, support tip, and bottom-side curved tip. Certain parameters are met by these embodiments of the tip of the present invention. For insertion, an embodiment of the present invention fits through an incision of a size described herein in further detail, without causing significant escape of viscoelastic. The tip design is atraumatic to the wound site, and an angle and manner of insertion by surgeon is intuitive. For deployment of the cutting element, the tip skirts around the nucleus along the rim of the anterior chamber, while simultaneously avoiding contact with the iris and endothelium. The tip is designed to guide the cutting elements along a side of the cataract while the tip skirts around the inner rim of the AC, avoiding contact with the corneal endothelium while maximizing the working space for the cutting elements to capture the cataract. For encapsulation, the tip supports the cutting elements as they encapsulate the nucleus. For positioning in the AC for the duration of use, the tip has to fit in the AC with the nucleus present. For the storage of cutting elements, the cutting element is stored within the tip in the retracted state during insertion and removal and with the exposed distal portion being flush with the surface of the tip. Additionally, the tip and its individual channels provide storage and repeated deployment for the cutting element without damaging/kinking the loops.

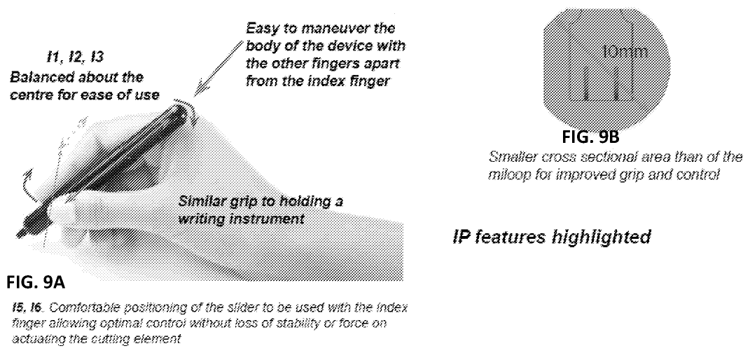

FIGS. 9A and 9B illustrate side and sectional views of a handle according to an embodiment of the present invention. As illustrated in FIG. 9A, the handle is balanced about a center axis for ease of use. The handle is structured for a similar grip to holding a writing instrument, such as a pen, pencil, or stylus. The configuration of the handle is to make it easy to maneuver the body of the device with the other fingers apart from the index finger, which is used to actuate the slider. The handle should allow for comfortable positioning of a slider actuating element to its maximum and minimum position with the index finger, allowing for optimal control of the device without loss of stability or force on the actuation of the cutting element. FIG. 9B shows a cross section of an exemplary handle. The area of the cross-section of the handle is smaller than other devices to allow for improved grip and control. In a preferred embodiment of the device, the handle is configured for single handed use, and is balanced about the inner web between the thumb and index finger. The design of the handle also prevents any dampening of force between the slider and cutting element in order to retain the haptic feedback from the tension in the cutting elements.

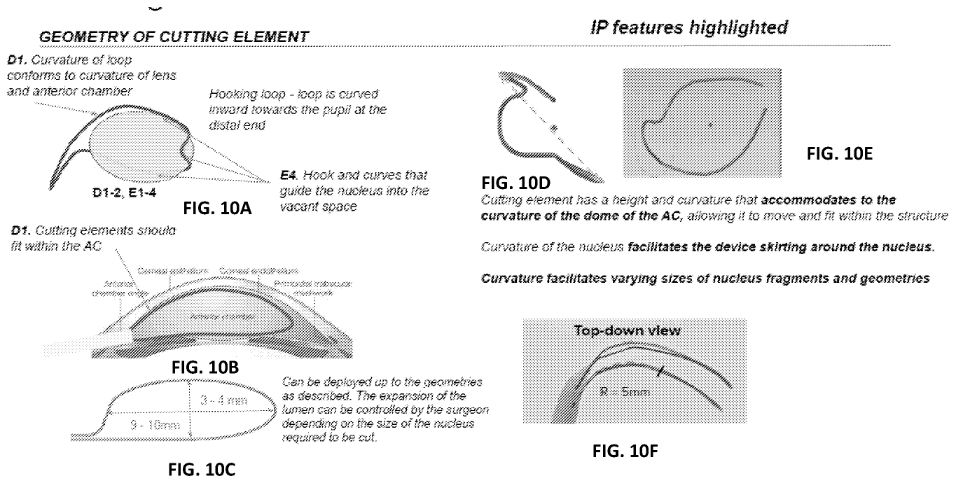

FIGS. 10A-10F illustrate views of the cutting element, according to the present invention. FIG. 10A illustrates a perspective view of a loop of the cutting element extending around the nucleus. The curvature of the loop is configured to conform to a curvature of the lens and fit within the AC so as to not damage the endothelial membrane or other delicate tissue within the AC. In some embodiments, the loop of the cutting element includes a hooking loop, which has an inward curvature at a distal end of the loop towards the center of the anterior chamber. The hooking loop and the curvature of the loop guide the nucleus into the vacant space. FIG. 10B illustrates a side view of a loop of the cutting element disposed within the AC. As illustrated in FIG. 10B the cutting element fits within the AC, and the curvature of the loop of the cutting element follows the curvature of the lens and the AC. FIG. 10C illustrates a side view of a loop of the cutting element. The loop can be deployed up to its geometries, as described herein. The expansion of the lumen defined by the loop and expanded when the loop is expanded is controlled by the surgeon. The expansion to an adequate lumen size is dependent on the size of the nucleus to be cut. The device of the present invention gives the surgeon control over the expansion size of the loop. Loop expansion can occur through forward translation of both ends of the loop wire or translation of only one end of loop wire, each of which require a different kind of loop curvature and curing in the case of nitinol (e.g., the former requires symmetrical curing of the top and bottom portions of the loop, and the latter relies on changing the curvature of the loop over the length of the loop to modify deflection and trajectory of the loops.

FIGS. 10D-10F illustrate views of loops of the cutting element. These views show that the cutting element has a height and curvature that accommodate the curvature of the dome of the AC, also illustrated in FIG. 10B. The height and curvature of the cutting element accommodates to the curvature of the AC, allowing the cutting element to fit within the structure of the AC. The curvature of the loop also facilitates the skirting of the loop around the nucleus so it can be captured and fragmented. The curvature of the loop also facilitates use with varying sizes of the nucleus, fragments of the nucleus, and different geometries of the particular nucleus of the patient. In addition, the angle between the two ends of the cutting element acts as a scoop during encapsulation while providing stability, keeping the nucleus in one plane and preventing it from tilting. This is enabled by the two ends acting on differing points along the surface of the cataract.

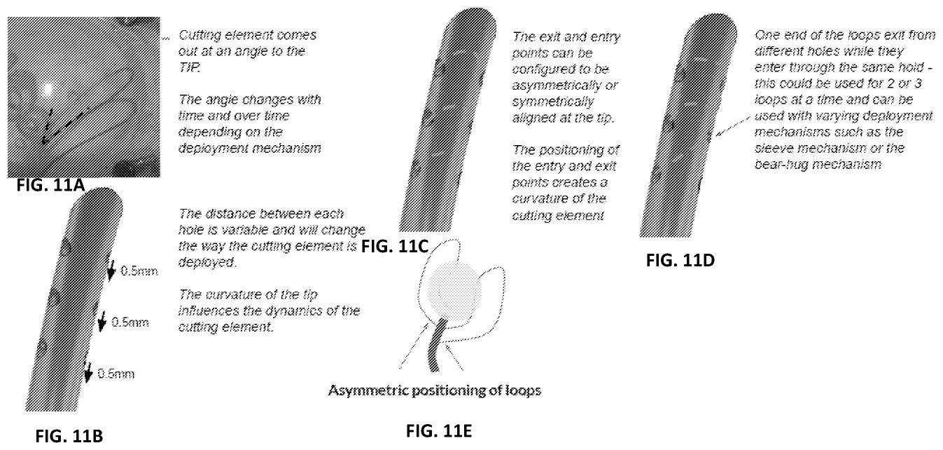

FIGS. 11A-11E illustrate views of the position of the cutting element as it exits a tip of the device, according to the present invention. FIG. 11A shows an image view of the cutting element being deployed from the tip of the device, in one exemplary embodiment of the device. As illustrated in FIG. 11A, the cutting element emerges from the tip of the device at an angle to the tip. The angle of the cutting element/s changes over the course of deployment of the cutting element and also depending on the particular deployment mechanism used in conjunction with the cutting element. FIGS. 11B-11D illustrate perspective views of exemplary tips of the device featuring exit holes through which the cutting element exits the tip of the device. The deployment and expansion of the cutting element is, in part, dependent on the positioning and configuration of these exit holes at the tip of the device. More particularly, the distance between each hole is variable and will change the way that the cutting element is deployed. The curvature of the tip also influences the dynamics of the cutting element and its deployment. As illustrated in FIG. 11C, the exit and entry points can be configured to be asymmetrically or symmetrically aligned at the tip. The positioning of the entry and exit points creates a curvature of the cutting element, which facilitates fit within the AC and also skirting of the nucleus. As illustrated in FIG. 11D, one end of each of the loops of the cutting element exits from a different hole and then the other end of each loop enters through the same hole. This exemplary embodiment could be used for 2 or more at a time and can be used with varying deployment mechanisms such as the sleeve mechanism or the bear-hug mechanism, which will be described in more detail, herein. FIG. 11E illustrates another exemplary embodiment with asymmetric positioning of the loops on the tip of the device. Asymmetric positioning can allow for scooping or capture of the nucleus from more than one side and create opposing forces on the nucleus to prevent the nucleus from gliding away from the loops during capture. The position of the loops exiting the tip can be manipulated to achieve the desired geometries of nucleus fragments, and to assist in deployment of the cutting element to the desired location on the nucleus.

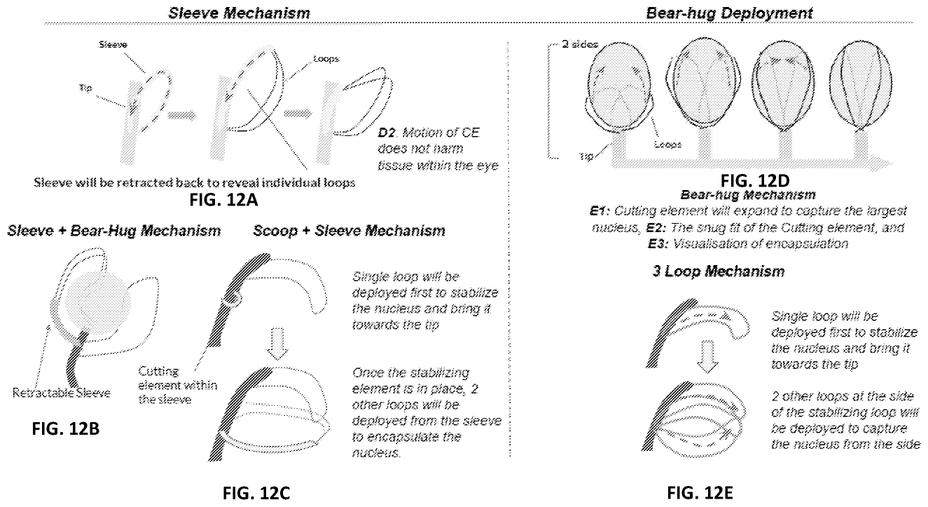

FIGS. 12A-12E illustrate views of motion of embodiments of the cutting element, according to the present invention. FIG. 12A illustrates a schematic diagram showing motion of a cutting element including a sleeve mechanism. The cutting element is deployed from the tip within the sleeve. The sleeve is then retracted back into the tip to reveal the individual loops of the cutting element. The individual loops then separate to a pre-determined distance based on the exit points from the tip, once the sleeve is retracted to a certain distance between them to encapsulate the nucleus. It should be noted that the motion of the cutting element does not harm the tissue of the eye in this or any of the other embodiments of the invention. The sleeve can be made of soft and flexible, yet non-expandable materials to allow for smooth motion up and down.

FIG. 12B illustrates a perspective view of a sleeve plus bear-hug mechanism. This embodiment includes a first pair of loops that are initially deployed in a sleeve, as described with respect to FIG. 12A and a third loop that is deployed from the other side and configured to hug the nucleus while enveloping the cataract within its loops by sliding along its surface in conjunction with the first pair of loops. This could have a dual actuation mechanism in which the loops and the sleeve are deployed together followed by the sleeve being retracted to reveal two loops which encapsulate the nucleus, Afterwards, all three loops can be retracted simultaneously.

FIG. 12C illustrates a schematic view of a scoop and sleeve mechanism. A single loop is deployed first to stabilize the nucleus and bring it towards the tip of the device. Once the stabilizing loop is in place, two additional loops are deployed with a sleeve. The sleeve is retracted, as described with respect to FIG. 12A to encapsulate the nucleus for fragmentation.

FIG. 12D illustrates a schematic view of a bear-hug deployment. A pair of loops forming the cutting element are deployed from the tip of the device. The pair of loops is then expanded to encapsulate the nucleus from the bottom-side facing surfaces of the nucleus. The bear-hug deployment allows for the cutting element to expand to the required dimension of the cataract and is therefore able to capture the largest sizes of cataract. The cutting element has a snug fit about the nucleus and also allows for clear visualization of the encapsulation of the nucleus and the location of the cutting elements during the procedure.

FIG. 12E illustrates a schematic view of a three loop mechanism. A single loop is deployed first to stabilize the nucleus and bring it toward the tip of the device. Additional loops, two, as illustrated in FIG. 12E are then deployed at the side of the stabilizing loop to capture the nucleus from the either of the sides, thus creating a web that prevents the nucleus from escaping through the loops by tilting and sliding along the plane it is on.

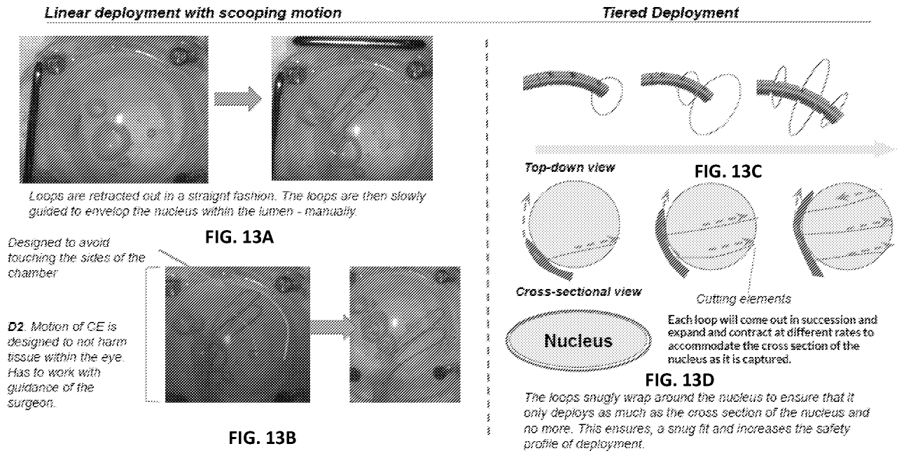

FIGS. 13A-13D illustrate views of motion of embodiments of the cutting element, according to the present invention. FIG. 13A illustrates a schematic diagram of the linear deployment of the cutting element along with a scooping motion to encapsulate the nucleus. In FIG. 13A, the loops are deployed outward in a straight fashion. The loops are then slowly guided to envelop the nucleus within the lumen defined by the loops. The loop guidance is executed manually and in some cases, a sinskey hook or other tool may be used to push the nucleus towards the loops while simultaneously expanding and guiding the loops inward.

FIG. 13B illustrates an additional schematic diagram of the linear deployment followed by scooping of the nucleus. The linear deployment combined with the scooping motion, assisted by the guided trajectory from the tip, is designed not to harm tissue within the eye.

The guidance of the surgeon is essential to this form of deployment and motion. FIG. 13C illustrates a schematic diagram of a tiered deployment of loops of the cutting element and FIG. 13D illustrates a top down view schematic diagram of the tiered deployment of the loops and subsequent encapsulation of the nucleus. As illustrated in FIGS. 13C and 13D, each loop will deploy in succession and expand and contract at different rates to accommodate the particular cross section of the nucleus, as it is captured. The loops snugly wrap around the nucleus to ensure that it only deploys as much as the cross section of the nucleus and not more. This ensures a snug fit and increases the safety profile of deployment.

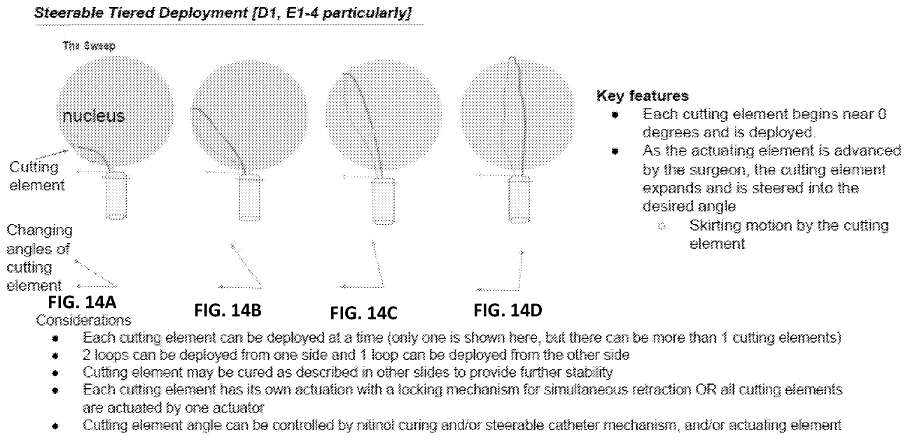

FIGS. 14A-14D illustrate views of a steerable tiered deployment of the cutting element, according to the present invention. In FIGS. 14A-14D the cutting element is deployed and is steered to the desired angle for encapsulation of the nucleus. Each cutting element begins near 0 degrees, as it is deployed. As the actuating element is advanced by the surgeon, the cutting element expands and is steered into the desired angle. This steering results in a skirting motion by the cutting element, as it encapsulates the nucleus. Each cutting element can be deployed one at a time or simultaneously at one time. While only one cutting element is shown here, there can be more than one cutting element. Two loops can be deployed from one side and one loop can be deployed from the other side. The cutting element may be cured as described further herein to provide additional stability. Each cutting element has its own actuation with a locking mechanism for simultaneous retraction. Alternately, all cutting elements are actuated by one actuator. The cutting element angle can also be controlled by nitinol curing and/or steerable catheter mechanism, and/or actuating element.

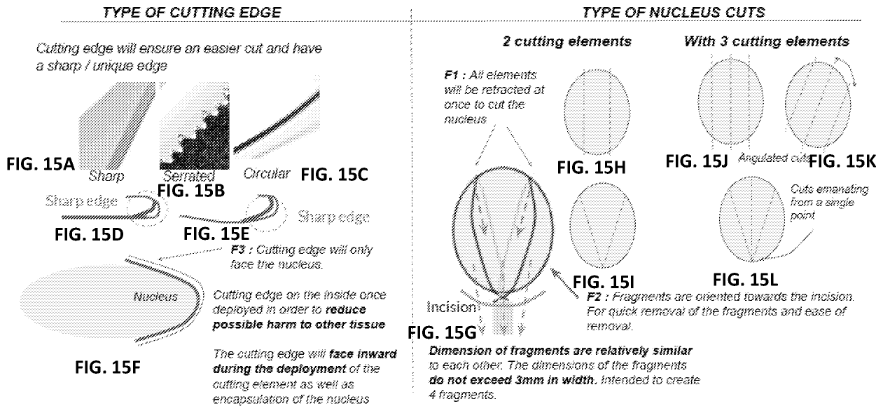

FIGS. 15A-15L illustrate views of the cutting element, according to the present invention. FIGS. 15A-15C illustrate different embodiments of a cutting blade of the cutting element. The cutting edge can remain unsharpened with a circular edge or the natural edge of the material used to make the cutting element, as in FIG. 15C. Alternately, the cutting element can have a sharpened or serrated edge, as in FIGS. 15A and 15B. The cutting edges could be of varying geometries to improve their effectiveness at cutting a hard nucleus. FIGS. 15D and 15E illustrate exemplary positions for a sharpened portion of the cutting element. FIG. 15F illustrates that the cutting edge, if sharpened, textured, serrated, or barbed, will only face the nucleus and not the tissue of the eye. The cutting edge is kept on the inside, facing towards the nucleus, through deployment and encapsulation in order to reduce possible harm to other tissue. In some embodiments, the aspect of the element facing the nucleus can be serrated, rough, or textured, while the aspect of the element facing the cornea is coated or covered with a protective material.

FIGS. 15G-15I illustrate a device with two loops and exemplary, resultant fragmentation of the nucleus, while FIGS. 15J-15L illustrate exemplary, resultant fragmentation of the nucleus with a three loop device. The cuts can be angulated or can arise from a single point. Fragments are oriented towards the incision. Orientation towards the incision allows for quick removal of the fragments and ease of removal.

The dimensions of the fragments created with this embodiment are relatively similar to one another. The dimensions of the fragments, preferably, do not exceed 3 mm in width, so that they can be removed from the same small incision that the device enters through. Size of the fragments, of course will vary based on the overall size of the cataract and the number of fragments. Here, the cataract nucleus is shown broken into four fragments. However, it is to be understood that the number of fragments is dependent on the embodiment of the device and the treatment circumstances, including the geometry, shape, and movement of the cutting elements, and all fragmentation patterns are considered included with the present invention. Generally, it is desirable to fragment the cataract into at least two fragments. Preferably, the cataract is broken into three to four fragments. In some embodiments and under certain treatment circumstances, it may be desirable or necessary to create more than three to four fragments, as would be known to or conceivable to one of skill in the art.

Generally, three to four fragments are created. Any material known to or conceivable to one of skill in the art can be used for the cutting element including, but not limited to a shape memory metal (i.e. nitinol) and polymers used in medical applications such as sutures (i.e. nylon, polyvinylidene fluoride (PVDF), polypropylene).

As described above, FIGS. 10A-10F; FIGS. 11A-11E; FIGS. 12A-12E; FIGS. 13A-13D; FIGS. 14A-14D; and FIGS. 15A-15L illustrate various possible implementations for a cutting element, according to the present invention, and applicable to any embodiment of the present invention. The cutting element can be utilized and manipulated in various forms to achieve its purpose of capturing and fragmenting the nucleus. Key factors for the cutting element are divided into two categories: 1) structure of the cutting element and 2) external factors influencing the cutting element. Structure of the cutting element includes: 1a) type of cutting edge, 1b) material composition, 1c) shape of the cutting element, 1d) curvature of the cutting element, 1e) thickness of the wire along the length of the element, and 1f) cross-sectional profile of the cutting element. External factors influencing the cutting element include: 2a) position along the tip, 2b) motion of the cutting element, 2c) positioning on the cataract associated with the geometry of fragments desired, 2d) exit and entry hole angulation on the tip, 2e) initial trajectory of the cutting elements, 2f) constraints on the wire (i.e. whether both ends move at once, independently or if one end is fixed) and 2g) geometry of the tip. For cutting element deployment, the expanded cutting element fits comfortably within the AC of the eye (does not scrape endothelium). The dynamics of the cutting element are configured in a way that does not touch tissue within the eye such as the iris or the corneal endothelium. For encapsulation, the cutting element expands large enough to capture even the largest lens type. The cutting element is snug to the nucleus while it is capturing the nucleus (influenced by shape and deployment dynamics of the cutting element) to prevent it from scraping the AC or iris. The cutting element is can be visualized during encapsulation. The cutting element can also guide the nucleus for capture and keep it in the same position relative to the other tissue while capturing it. With respect to fragmentation, the goal is to fragment the cataract in one motion to get, preferably, 3-4 fragments of similar dimension. Additionally, it is ideal to align the fragments to the incision and removal site while doing so. The cutting edge is also designed to be positioned on the inner surface of the cutting element to avoid contact with tissue of the eye.

Further considerations for the cutting element, include that it expands to accommodate the largest nucleus types, such as those up to 10 mm in diameter and up to 3.5 mm in height. The trajectory of the cutting element is designed to encapsulate the nucleus as it is deployed and fragment the nucleus. The surgeon has complete control over the movement of the cutting element within the AC and the cutting element remains visible or intuitively visible throughout the procedure. It should be noted that these features of the cutting element can be used individually or in concert for any of the embodiments of the present invention.

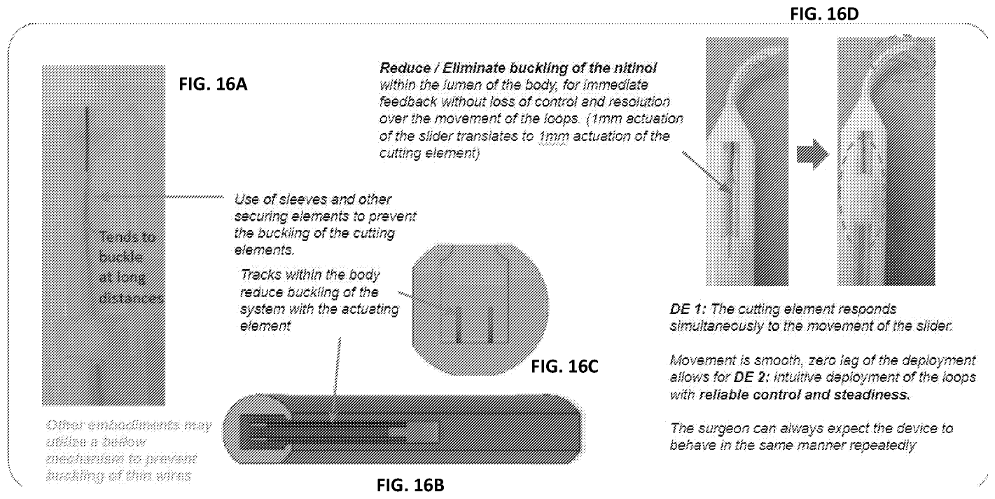

FIGS. 16A-16D illustrate views of an actuating element for the cutting element, according to an embodiment of the present invention. FIG. 16A illustrates a top-down view of the cutting element disposed within the handle of the device. The cutting element is surrounded by a sleeve. The use of a sleeve, as illustrated in FIG. 16A, prevents buckling of the cutting element. FIGS. 16B and 16C illustrate a track within the body of the handle. The track also reduces buckling of the cutting element while being deployed by the actuating element. Other embodiments may use a bellow or other mechanism to prevent internal buckling during deployment. There may also be a mechanical mechanism or material, such as memory foam or other compressible material that rests above or around the wires to provide a tamper for the wires, preventing buckling while allowing smooth translation of the slider back and forth. FIG. 16D shows a schematic diagram of the actuating element being advanced from the proximal to the distal position. This advancement of the slider of the actuating element generates simultaneous movement of the cutting element. The movement is smooth and has zero lag. The slider and corresponding simultaneous movement of the cutting element also allows for intuitive deployment of the loops of the cutting element with reliable control and steadiness. Therefore, the surgeon can always expect the device to respond in the same manner even with repeated use.

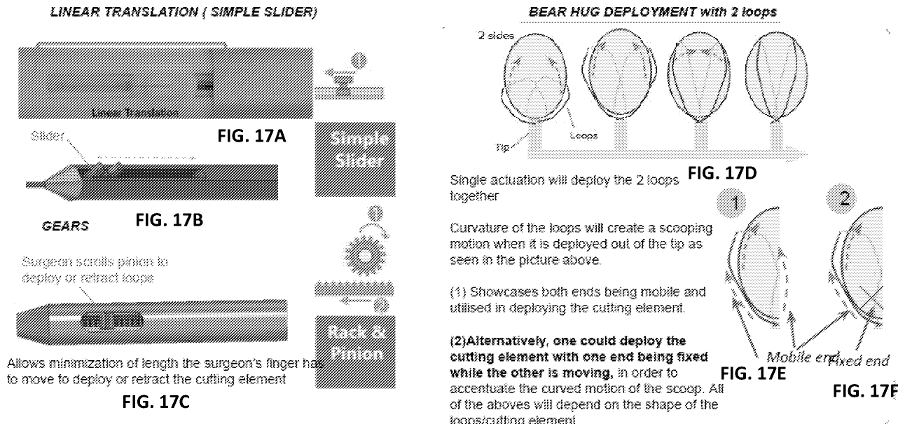

FIGS. 17A-17F illustrate views of a single actuation element, according to the embodiment of the present invention. FIGS. 17A-17C illustrate views of a slider actuation element, according to an embodiment if the present invention. The simple slider of FIGS. 17A and 17B allows for linear translation along the axis. In the rack and pinion embodiment of FIG. 17C, the surgeon scrolls the pinion to deploy or retract the loops of the cutting element. The rack and pinion design allows for the minimization of the length the surgeon's finger has to travel to deploy or retract the cutting elements.

FIGS. 17D-17F illustrate views of the actuation of a two loop design configured to hug the cataract. Single actuation deploys the two loops together. Curvature of the loops creates a scooping motion, when it is deployed out of the tip. FIG. 17E shows both ends being mobile and utilized in deploying the cutting element. FIG. 17F alternatively, shows that one could deploy the cutting element with one end being fixed while the other is moving, in order to accentuate the curved motion of the scoop. All of the above will depend on the shape of the loops/cutting element.

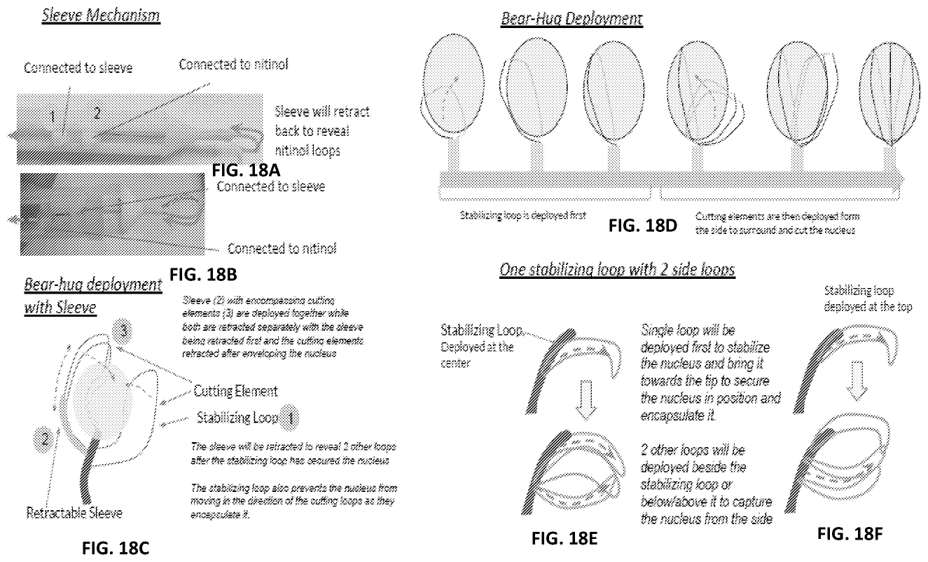

FIGS. 18A-18F illustrate views of a multi-actuation element, according to an element of the present invention. FIGS. 18A and 18B illustrate views of an actuation method with multiple actuators. FIG. 18C illustrates a multi-loop design with a retractable sleeve designed for hugging the nucleus. FIG. 18D illustrates a flow diagram of deployment of a multi-loop design for hugging the nucleus. This embodiment is at times referred to as the bear hug design. FIGS. 18E and 18F illustrate schematic diagrams of stabilizing loop deployment. FIGS. 18A and 18B illustrate views of an actuation method with multiple actuators and a sleeve mechanism. One actuator is connected to the cutting element and one actuator is connected to the sleeve element. The sleeve element will retract back into the tip of the device to reveal the loops of the cutting element. FIG. 18C illustrates a multi-loop design for hugging the nucleus of the cataract with a retractable sleeve. Sleeve (2) and an encompassing cutting element (3) are deployed together while both are retracted separately with the sleeve being retracted first and the cutting elements retracted after enveloping the nucleus. The sleeve is retracted first to reveal the other loops after the stabilizing loop has secured the nucleus. The stabilizing loop also prevents the nucleus from moving in the direction of the cutting loops as they encapsulate it.

FIG. 18D illustrates a flow diagram of deployment of a multi-loop design for hugging the nucleus. FIGS. 18E and 18F illustrate schematic diagrams of stabilizing loop deployment with two side loops. In FIGS. 18D-18F, the stabilizing loop is deployed first. The cutting elements are then deployed from the side to surround and cut the nucleus. The stabilizing loop is deployed from the center of the tip of the device, in FIG. 18E and from the top of the tip of the device in FIG. 18F. The single stabilizing loop is deployed first to stabilize the nucleus and bring it towards the tip of the device to secure the nucleus in position and encapsulate it. Two other loops are then deployed beside the stabilizing loop or below/above it to capture the nucleus from the side.

With respect to FIGS. 16A-16D; 17A-17F; and 18A-18F, the actuating element has several similarities throughout. There is no lag between movement of the actuating mechanism and the movement of the cutting element, thereby allowing the surgeon to receive almost immediate haptic feedback and achieve high resolution and control over the movements of the device within the eye. The direction of actuating element's movement is intuitive with respect to the deployment of the loops. The type of mechanism used is largely dependent on the type of cutting element and its deployment. Depending on the mechanisms, more than one actuator could be used to control multiple moving parts. The force provided by the user has to translate to the cutting element without loss of impulse corresponding to the haptic feedback of the translational force required by the surgeon he movement of the actuating mechanism and the cutting elements is also intuitive to the surgeon and based on the manner the cutting element moves within the anterior chamber.

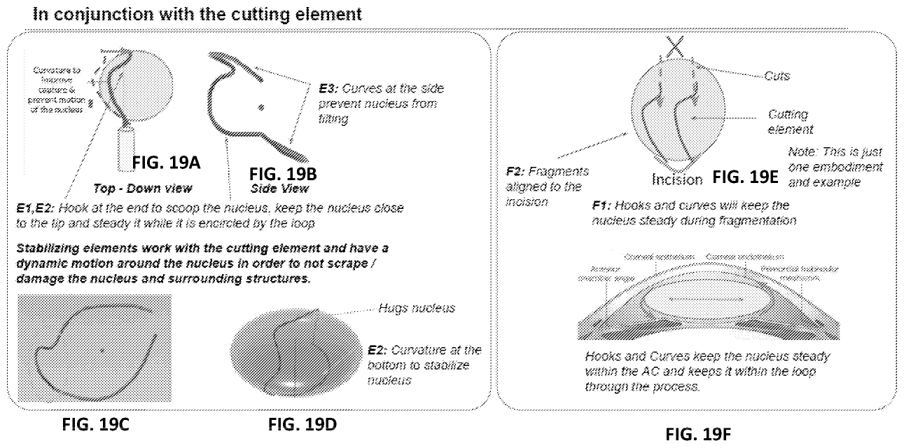

FIGS. 19A-19F illustrate views of a stabilization element used in conjunction with the cutting element, according to an embodiment of the present invention. FIGS. 19A-19F illustrate views of curves in the loop of the cutting element to improve capture and prevent tilting of the nucleus. The hook at the end of the loop is configured to scoop the nucleus, keep the nucleus close to the tip, and steady it while it is encircled by the loop. Stabilizing elements work with the cutting element and have a dynamic motion around the nucleus in order to not scrape or damage the nucleus and surrounding structures. The hooks and curves of the loop help to keep the nucleus steady during fragmentation and within the loop throughout the whole process.

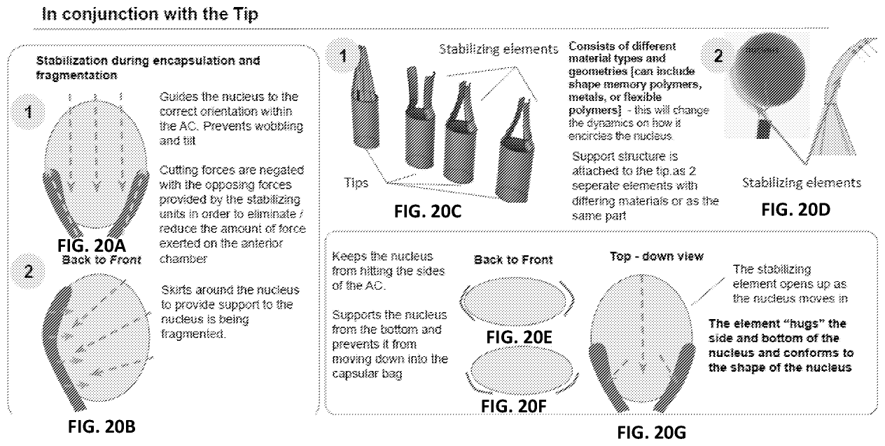

FIGS. 20A-20G illustrate views of a stabilization element use in conjunction with the tip of the device, according to an embodiment of the present invention. Flaps on the tip of the device provide stabilization during encapsulation and fragmentation. The flaps guide the nucleus to the correct orientation within the AC. The flaps prevent wobbling and tilt. Force created by the cutting elements are opposed by the normal forces of equal magnitude and direction provided by the stabilizing units such as the edge of the tip that the cataract is pulled towards in order to eliminate/reduce the amount of force exerted on the anterior chamber. The tip flaps skirt around the nucleus to provide support to the nucleus that is being fragmented. Support structure is attached to the tip as two separate elements with differing materials or as the same part with the same materials. The flaps keep the nucleus from hitting the sides of the AC and support the nucleus from the bottom to prevent it from moving down into the capsular bag. The stabilizing element opens up as the nucleus moves in between the flaps. The element “hugs” the side and bottom of the nucleus and conforms to the shape of the nucleus. The stabilizing element and its flaps can be formed from different material types and geometries, which can include shape memory polymers, metals, or flexible polymers. The material used changes the dynamics of how the flaps encircle the nucleus.

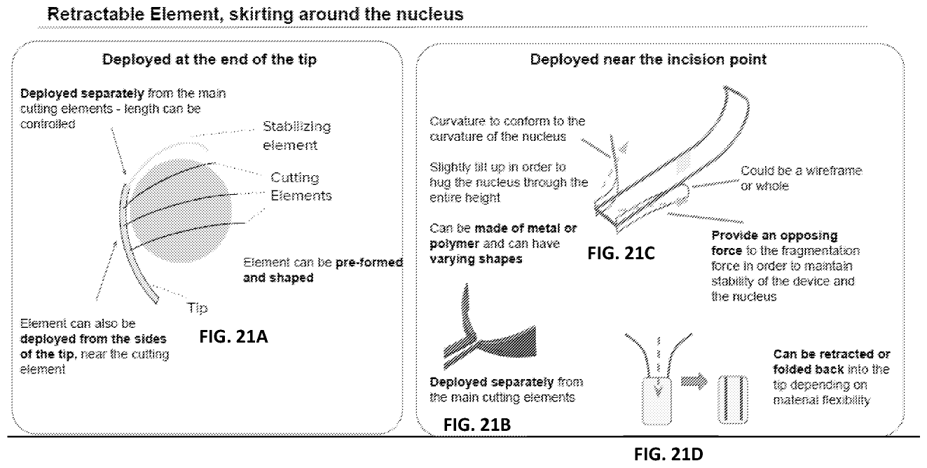

FIGS. 21A-21D illustrate a retractable stabilization element that skirts around the nucleus, according to an embodiment of the present invention. FIG. 21A illustrates a stabilizing element deployed at the tip of the device. The stabilizing element of FIG. 21A is deployed separately from the main cutting elements. In this way the length of the stabilizing element can be controlled and customized to the nucleus of the cataract. Alternately, this stabilizing element can be deployed from the sides of the tip, near the cutting element, or at the base of the cutting element. The element can be pre-shaped to fit within the AC and around cataract nuclei.

In FIGS. 21B-21D, the stabilizing elements are deployed from near the base of the tip and the incision point. The curvature is configured to conform to the curvature of the nucleus. The stabilizing elements shown in these figures slightly tilt up in order to hug the nucleus through its entire height. The stabilizing elements can be made of metal or polymer and can have varying shapes. Similarly, to FIG. 21A, the stabilizing elements can be deployed separately from the main cutting elements. The stabilizing elements could be a wire frame or solid structure. The stabilizing element provides an opposing force to the fragmentation force in order to maintain stability of the device and the nucleus. The stabilizing element can be retracted or folded back into the tip depending on material flexibility.

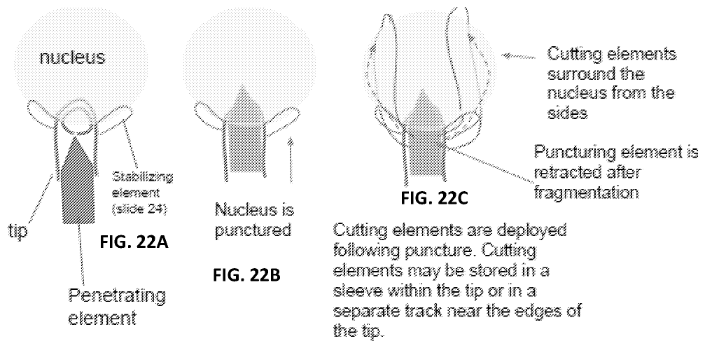

FIGS. 22A-22C illustrate a retractable, penetrating stabilization element, according to an embodiment of the present invention. FIG. 22A illustrates a schematic diagram of a penetrating stabilization element being deployed from the tip of the device. As illustrated in FIG. 22A the stabilization element includes flaps as described above, as well as the penetrating stabilizing element. In some embodiments, the device may only include the penetrating stabilizing element. A single penetrating element is shown in FIG. 22A. However, multiple, thinner or finer elements can also be used to carry out the same function. Indeed, any implementation of the penetrating stabilizing element known to or conceivable to one of skill in the art can be used. As illustrated in FIG. 22A, the cutting elements are deployed following puncture. Cutting elements may be stored in a sleeve within the tip or in a separate track near the edges of the tip. The cutting elements surround the nucleus, which has been punctured by the stabilizing element. The nucleus is then fragmented, and the puncturing element is retracted after fragmentation.

FIGS. 22B and 22C illustrate side views of exemplary designs of the penetrating element. In FIG. 22B, the penetrating element is smooth and sharp at the end, like a needle. In FIG. 22C, the penetrating element is spiraled like a corkscrew. These exemplary designs are not meant to be considered limiting and any design for the penetrating tip known to or conceivable by one of skill in the art could be used.

Further with respect to penetrating stabilizing elements, the penetrating element can come from the center of a tip that deploys curved cutting elements, or it can come from the center of the curvature of a curved tip. The penetrating element may be connected to a push-pen like actuator which has a locking and unlocking mechanism. In some embodiments, the penetrating element may be in the form of a corkscrew and connected to a dialer to actuate the element. As noted above, the penetrating element can be used along with another stabilizing element on the tip to provide further stability during puncture or may be used with a routine side port instrument like the sinskey hook. In other embodiments, the penetrating element may be linearly translated forward or be rotated in a radial trajectory (in the anterior-posterior, proximal-distal plane) as it is deployed. Again, it should be noted that these are merely examples of potential penetrating elements and associated features, this description is not exhaustive, and any design or features for the penetrating element known to or conceivable to one of skill in the art can be used.

With respect to FIGS. 19A-19F; 20A-20G; 21A-21D; and 22A-22C, these figures show elements that stabilizes the nucleus in order to capture and fragment it. The stabilization element(s) are used for encapsulation of the nucleus and for guiding the nucleus within the lumen of the loops, and in some embodiments, guiding the cutting elements in encapsulating the cataract while maintaining the position of the cataract. They could also be used to assist maintaining the alignment of the nucleus during fragmentation. There are four broad categories of stabilization elements: 1) a stabilizing element that is combined with the cutting element, 2) a stabilizing element that is combined with the tip, 3a) a retractable stabilizing element that functions around the nucleus and 3b) a retractable stabilizing element that penetrates the nucleus. The stabilization elements of these figures provide support during encapsulation by preventing the nucleus from wobbling during encapsulation, preventing the nucleus from slipping out of the cutting element, and preventing the nucleus from tilting (safety+ensure better cut) as element is capturing and retracting. Additionally, the stabilization elements of these figures also provide support during fragmentation by keeping all of the fragments steady in a particular orientation while the loops are retracted to get smooth, straight and clear cuts, and aligning the fragments to the incision. The penetrating element designs of each of these figures provides a surface area for adequate stabilization and curvatures along the cutting elements to steady the nucleus during the procedure. The stabilization element may be combined with other elements such as the tip of the cutting element to provide further stability. Deployment can be static or dynamic. The material chosen for any of the implementations of the stabilization elements shown in these figures should provide grip on the nucleus and change dynamic motion dependent on material flexibility/pliability.

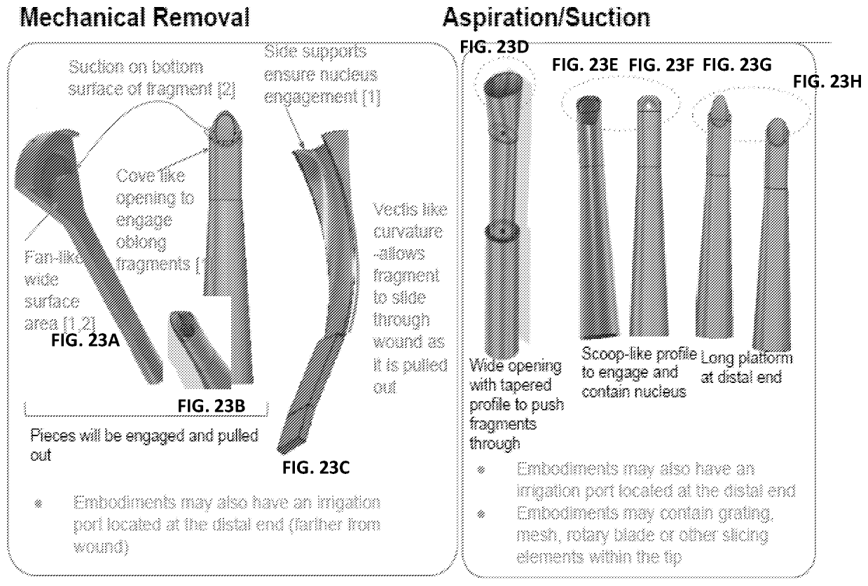

FIGS. 23A-23H illustrate views of tips designed for removal of cataract fragments, according embodiments of the present invention. These removal tips are positioned in the AC underneath the fragments of the nucleus. FIGS. 23A-23C illustrate tips for mechanical removal of cataract nucleus fragments. FIGS. 23A and 23B illustrate tips that provide suction on the bottom surface of fragments. The fragments are engaged and then pulled out of the eye through the incision. In FIG. 23A, the tip has a fan-like, wide surface area, and in FIG. 23B, the tip has a cove-like opening to engage fragments. FIG. 23C illustrates a removal tip design that provides side support to ensure nucleus engagement, and a curvature allows for fragments to side through the incision as they are removed. The embodiments shown in FIGS. 23A-23C can include an irrigation port located at the distal end. Irrigation can be in the form of manual or continuous irrigation.

FIGS. 23D-23H illustrate views of tips for aspiration or suction-based removal of the fragments. As illustrated in FIG. 23D, the tip can have a wide opening, with a tapered profile to push the fragments through. FIGS. 23E and 23F illustrate a scoop-like profile to engage and contain the fragments while providing a base to position the fragments before removal through the lumen or while engaging the fragments at the lumen if the fragment is larger or has an irregular shape compared to the lumen. Stabilizing the fragment enables the surgeon to be more effective in targeting the fragment and utilizing the appropriate amount of suction force, thus reducing the risk of suctioning out other material, such as viscoelastic within the eye that keeps the eye pressurized during the procedure

FIGS. 23G and 23H illustrate a long platform at the distal end. Suction can take the form of a manual syringe, an automated syringe, an aspiration bulb, a vacuum pump, venturi suction, or any other form of method of delivery of suction known to or conceivable to one of skill in the art. The suction or pulling force can be continuous, periodic, or delivered in pulses. The embodiments in FIGS. 23D-23H can also have an irrigation port located at the distal end. These embodiments may contain grating, mesh, or a rotary blade or other slicing element within the tip that generate smaller fragments in order to ease suction and reduce the chance of the fragment clogging the lumen down the tube.

The removal tips illustrated in FIGS. 23A-23H could be integrated with a fragmentation device having a disposable collection bin in the rear end of the handheld tool. Removal tip could also be part of a separate device with its own disposable collection bin. It should be noted that the removal tips shown in FIGS. 23A-23H are merely examples and are not meant to be considered limiting. Any implementation of the removal tip known to or conceivable by one of skill in the art could also be used.

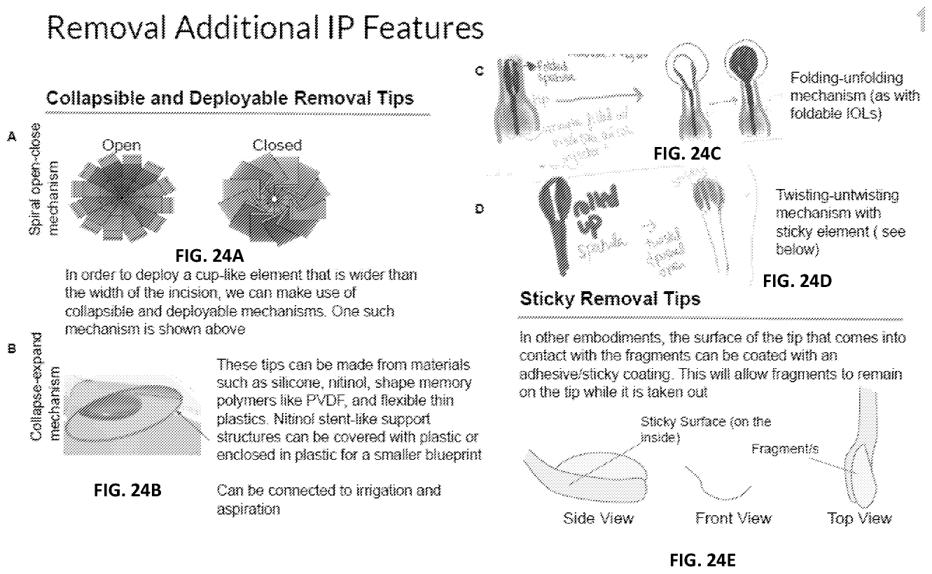

FIGS. 24A-24E illustrate views of additional tip designs configured for removal of cataract fragments, according to embodiments of the present invention. FIGS. 24A-24D illustrate collapsible and deployable removal tips. FIG. 24A illustrates a spiral open-close mechanism. In order to deploy a cup-like element that is wider than the width of the incision, a collapsible and deployable or expandable mechanism can be used. FIG. 24B illustrates a collapse-expand mechanism that creates a funnel to suction the whole cataract that tapers off along the length of the funnel in order to crush the cataract as it is being brought through the funnel. FIG. 24C illustrates a folding and unfolding mechanism (as with a foldable intraocular lens), and FIG. 24D illustrates a twisting and untwisting mechanism with a sticky element.

FIG. 24E illustrates an exemplary embodiment incorporating a sticky element. In any of the embodiments described above, in addition to FIG. 24E, the surface of the tip that comes into contact with the fragments can be coated with an adhesive or other sticky/gripping coating. This coating will allow fragments to remain adhered to the tip while the tip is removed from the incision. These removal tips can be made from materials such as silicone, nitinol, shape memory polymers like PVDF, flexible, thin plastics, or any other suitable materials known to or conceivable to one of skill in the art. Nitinol stent-like support structures can be covered with plastic or enclosed in plastic for a smaller blueprint. These removal tips can also be coupled to irrigation and/or aspiration.

With respect to the removal tips illustrated in FIGS. 23A-23H and 24A-24E the removal tips are particularly designed/shaped to grasp and collect nuclear fragments. In combination, the tip can be utilized with a variety of collection mechanisms. There are two kinds of approaches, each resulting in a different kind of removal tip design: 1) the mechanical-based removal, 2) aspiration/suction tip. The mechanical removal design utilizes the curvature of the tip to hold the fragment (in combination with irrigation/aspiration in some cases), to pull out fragments through the wound, while the aspiration/suction designs involve suctioning fragments through the tip to a collector. Deployment mechanisms and other features of the tip: 1) collapsible/expandable mechanism, 2) twist open-close mechanism, 3) sticky adhesion, 4) folding-unfolding mechanism. The system has to be able to engage the fragment and maintain grasp of the engaged fragment until the fragment is removed through an external collector or delivered through the incision. The system has to exert enough force to deliver fragments through a 3 mm wound or through a collection tip which fits within the 3 mm wound. The removal procedure (mechanical, pneumatic, or otherwise) should not cause AC collapse. Therefore, the removal tip has to move in a manner that does not disrupt other ocular tissue including the iris, endothelium and posterior capsule.

Size of the fragments, of course will vary based on the overall size of the cataract and the number of fragments. However, it is to be understood that the number of fragments is dependent on the embodiment of the device and the treatment circumstances, including the geometry, shape, and movement of the cutting elements, and all fragmentation patterns are considered included with the present invention. Generally, it is desirable to fragment the cataract into at least two fragments. Preferably, the cataract is broken into three to four fragments. In some embodiments and under certain treatment circumstances, it may be desirable or necessary to create more than three to four fragments, as would be known to or conceivable to one of skill in the art. The surgeon is also able to orient the fragments, such that a cross section of the fragment with the smallest width is oriented parallel to the incision.

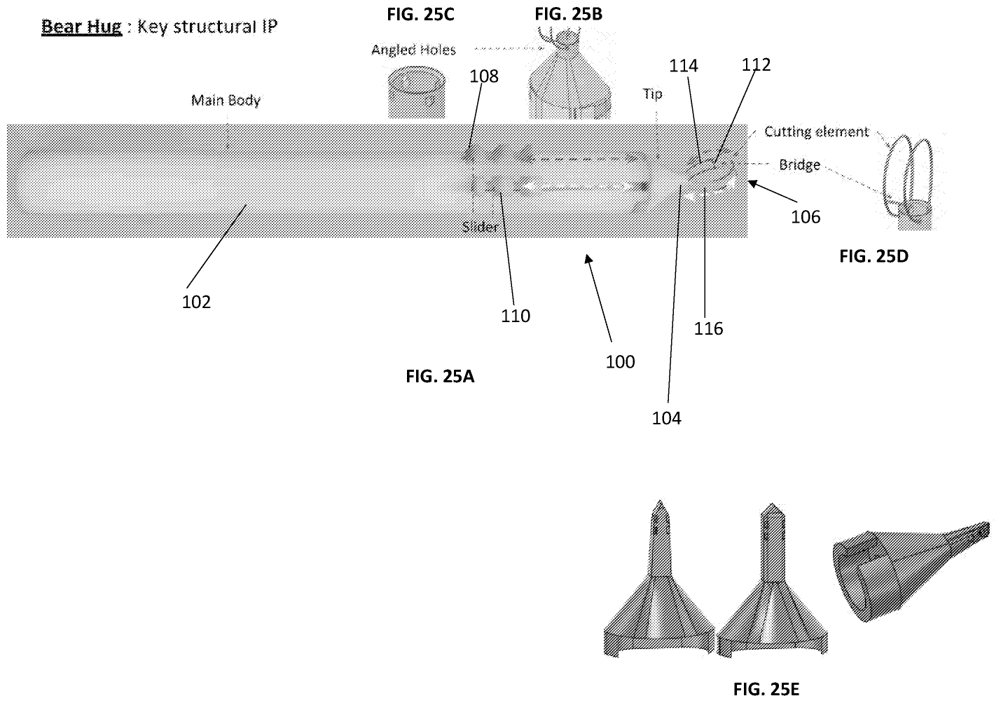

FIGS. 25A-25D illustrate views of a device designed to hug the nucleus, according to an embodiment of the present invention. FIG. 25A illustrates a perspective view of a bear-hug device, according to the present invention. FIGS. 25B-25D illustrate perspective views of details of the tip and cutting element. The bear-hug device 100 includes a handle 102, a tip 104, and a cutting element 106. The handle 102 houses the sliders 108, 110 and internally houses the mechanisms for deploying the cutting element 106. A bridge 112 couples the loops 114, 116 of the cutting element 106. The sliders 108, 110 provides adequate grip for the surgeon, and carries out linear translation of each end of the cutting element. The slider provides haptic feedback through friction between the slider and main body, resulting in almost immediate feedback to the surgeon. Additionally, the slider locks the cutting element position in place when not in use. The parallel slider 108 actuates the top ends of both 114 and 116 while slider 110 actuates the bottom end of 114 and 116. When slider 108 is moved, the top end of 114 and 116 move together with the bridge 112, with bridge 112 being connected to the top ends of the loops. In some embodiments, both ends of both cutting elements are actuated with a single slider, thus, deploying the cutting elements simultaneously in a symmetrical fashion out of the tip. The connection of the bridge to the loop is fixed and does not move during the procedure.

Tips that have a horizontal side profile, with some tips having a taper in a range of approximately 1 degree to approximately 3 degrees, are used to deploy the cutting elements in a straight trajectory along a straight, single plane. Exit and entry holes for these cutting elements are located along the tip at a minimum of approximately 0.2 mm from the distal end. Tips have variable hole sizes, preferably ranging from approximately 0.3 mm to approximately 1.5 mm inner diameter, and are preferably positioned between approximately 0.2 mm to approximately 3 mm from the distal end of the tip.

In some embodiments, a scoop was added to the bottom of the tip to further stabilize and support the nucleus and open up the wound for easier entry of the remainder of the tip. The scoop can vary in length, shape and cross-sectional area covered by the lumen of the scoop depending on the embodiment of the invention and the intended application. The scoop can be hollow, partially filled, netted with a variety of patterns or solid. The scoop can be a passive stationary element, or it can be a deployable and retractable element, either deployed independently from the loops, together with the loops or together with one end of the loops. It should be noted that these scoop designs are included simply by way of example and any other suitable configuration for the scoop known to or conceivable to one of skill in the art could also be used.

Further, with respect to FIG. 25A, the tip 104 houses the cutting element 106 and bridge 112 and is the site of deployment of both elements. The bridge 112, in embodiment shown in FIG. 25A is flush with the surface of the tip in its resting state (prior to deployment). Bridge 112 enables the cutting elements 114 and 116, to traverse the sides of the cataract, maintain an inward force on the surface of the cataract as they are deployed and translated, and engulf the cataract within their lumen. The cutting element 106 captures the cataract within the lumen defined between the loops 114, 116. The cutting element 106 is used to fragment the cataract. Additionally, the internal facing edges of the cutting element stabilize the cataract as they move alongside it, preventing side-side and the anterior-posterior movement during deployment. The bridge 112 enables dynamic deployment of the cutting element 106. The bridge 112 also enables nucleus capture. Bridge 112 keeps the top portion of loop 114 connected to the top portion of loop 116. Bridge 112 is connected to loop 114 at the same position it is connected to loop 116, this creating a straight symmetrical joint between the two loops. The bridge is docked into a slit within the tip 104 of the device or docked flush with the tip surface for a smooth entry through the incision. The bridge 112 enables the cutting element 106 to hug the nucleus as it is deployed, creating an inward force on the cataract by retaining the tension between the loops by keeping the bottom ends of loops 114 and 116 spread apart. The bridge 112 also cuts the central fragment into two pieces based on the final placement of the bridge on the surface of the cataract as defined by the surgeon and its corresponding retraction trajectory.

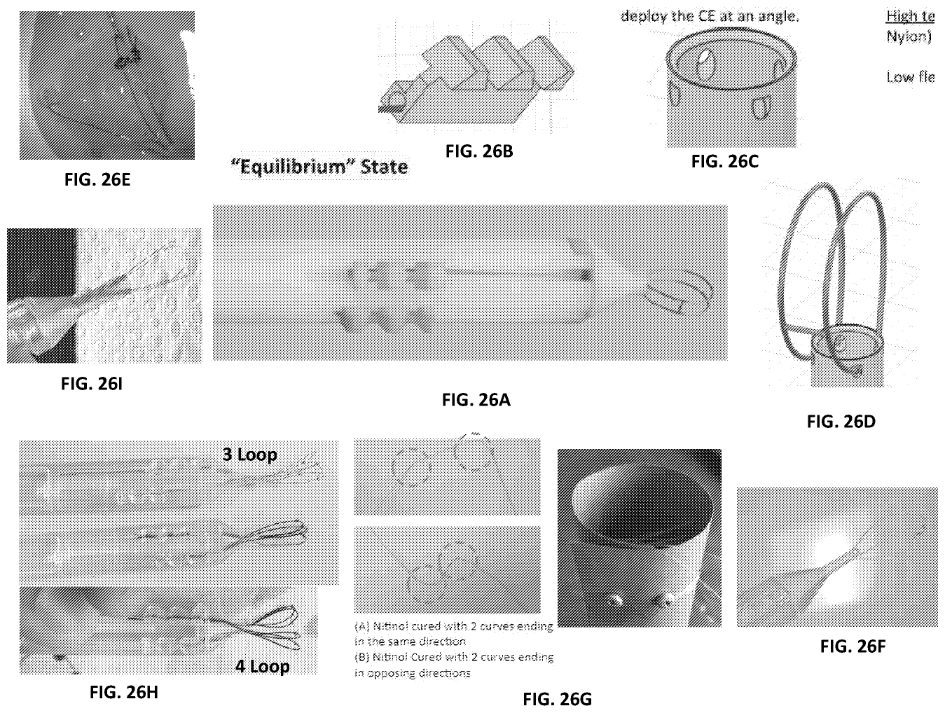

FIGS. 26A-26I illustrate additional views of a device designed to hug, fragment, and remove fragments of the nucleus, according to an embodiment of the present invention. FIG. 26A illustrates the device in an equilibrium state where the cutting loops are in their natural state, deployed straight without a body between the loops to create tension in the bridge. FIG. 26B illustrates a perspective view of a slider of the device. FIG. 26C illustrates a tip of the device and the angled exit holes for the loops of the cutting element, and FIG. 26D illustrates a tip of the device with the cutting element deployed. Each slider is connected to either of the ends of the loops, with the top end of loops connected to slider and the bottom ends of loops connected to slider, giving the user control over the deployment of either end of the loops. The slider also includes triangular etching for grip, though any pattern or method of providing grip known to or conceivable to one of skill in the art can also be used. The tip defines a circular, elliptical, or quadrilateral lumen that is approximately 2 to approximately 3 mm in diameter. Alternately, the tip can have a maximum height of approximately 2.5 mm and width of approximately 3 mm for a 3 mm incision. The ratio as prescribed can be translated for any size of incision, with greater flexibility in range for larger incisions. The lumen can be open or filled.

As illustrated in FIGS. 26C and 26D the tip defines four equidistant holes on the same horizontal plane. There are two holes on the top and two holes on the bottom. The holes are approximately 0.5 mm in diameter and approximately 0.5 mm from the distal end of the tip. The holes are angled at approximately 0 degrees to approximately 60 degrees from the normal to deploy the cutting element at an angle. The holes can also have an angulation to the side of approximately −90 degrees to approximately 90 degrees. In other embodiments the tips can also have variable geometries and the ends of the tip could be elliptical, circular, square or rectangular with the holes on the tips being circular or rectangular with lengths of up to 1 mm to widths of up to 0.5 mm.

The distal end of the tip could be made sharp into a triangular structure that is symmetrical to the mid-plane of the nucleus. This creates another fracture site when the cutting elements retract after capturing the cataract. This fracture site leads to a split in the nucleus, thus generating two additional fragments.

Embodiments utilizing a single bridge (with two or three loops of variable material) shown in FIGS. 26A and 26E were used in conjunction with straight tips to engage the nucleus from the front instead of the side. The bridge composed of nylon or polypropylene glides over the nucleus due to the material's low coefficient of friction. The bridge was deployed over the nucleus instead of under to improve visibility of the bridge and provide control over its trajectory by enabling the user to determine its location at any time. This method of deployment is not exhaustive, and the user may find alternative deployment methods to encapsulate the cataract not described or shown in the figures. FIG. 26F illustrate an embodiment with loops that are barbed that are connected to a bridge made from polypropylene. FIG. 26G illustrates the cured nitinol loops with a single or multiple curvatures along its length with each curvature having a predefined radius of curvature and distance between each curve. In some embodiments, the three or four loops utilize two or three bridges respectively with each bridge connecting to two loops as shown in FIGS. 26H and 26I.

With respect to FIGS. 26A and 26D, the cutting element is preferably formed from 0.003″-0.004″ Nitinol cured in a circular fashion of 5 cm-4 cm diameter. Alternately, nylon 4-0 and 6-0 sutures (approximately 0.15-0.05 mm in diameter) can be used. With respect to any of the cutting loops of the present invention, it is possible to use a number of different materials with varying diameters and cross-sectional shapes. In some instances, it is possible to use super-elastic nitinol wire with varying cross-sectional diameter and geometry. In some instances, the cross-sectional diameter can vary along the length of the wire. The diameter of the wire can be between approximately 0.003 inches to approximately 0.005 inches in diameter. The cross-sectional shape of the super-elastic nitinol wire can be round, flat or any other geometry known to or conceivable to one of skill in the art. The loops can alternately be formed from any other material known to or conceivable to one of skill in the art, including but not limited to other shape memory metals, steel, metal alloys, nylon, polymers, or other plastics or elastomers. If nylon is used, it can take the form of a nylon thread in a preferable range between 6-0 to 2-0 suture sizes, which is approximately 0.07 mm to approximately 0.3 mm in diameter.

The material for the cutting element has super elastic properties, a high tensile strength, and a low flexural modulus. If nylon is used, it does not deform when bent. The nitinol or the nylon have a smooth surface and circular cross-section. The bridge is formed from of nylon 6-0 or polypropylene 6-0. The intersection between the cutting elements and the bridge is formed through adhering a knot to the particular position on each of the cutting element loops. This knot can be adhered to the cutting element via mechanical, pressure, thermal, or chemical means, or a combination of these methods or others. The smooth nature of the material of the bridge allows it to dock easily with the tip. The material of the bridge should also have a low flexural modulus, so that it doesn't lose its shape when it's bent. The bridge can be composed of a variety of polymers and elastomers, including but not restricted to materials found in sutures and shape memory devices. The bridge can preferably have a variable length from approximately 2 mm to approximately 3.5 mm. However, in some embodiments a bridge of length between approximately 0 mm to approximately 12 mm could also be used. In this embodiment, the bridge non-elastic and retains its shape and position throughout the procedure. The bridge is placed symmetrically on both loops. The bridge is adjustable over the length of the cutting element from end to end, adjusted by the movement of the sliders as they move each of the corresponding ends of the cutting element, and is adhered to the cutting element, i.e. the bridge is immobile on the loops of the cutting element. Additionally, the bridge is repositionable up and down the loops relative to the distance between the top and bottom as per the movement and position of the sliders. This changes where the restriction of the loops is. In some embodiments utilizing a polymer cutting element, a three loop (cutting element) structure is utilized to capture the nucleus using two bridges, one between each loop, or a single bridge, between either loops on either end, with the nucleus being enveloped through either of the loops. This allows for fragmenting the cataract into 4-6 pieces. All the cutting elements are deployed from holes of variable sizes from the tip. In other embodiments, a four loop (cutting element) structure is utilized to capture the nucleus with three bridges of similar length, connecting all four loops together. The bridges connect each loop with the adjacent loop at a point along the length of the loop. The four loop structure is able to fragment the cataract into 5-8 pieces. The polymer-based loops enable smooth deployment, gliding along the surface of the cataract, and has enough resistance to hug the nucleus. The cutting element could be made of barbed sutures, allowing the cutting element to dig into the nucleus and create additional fracture sites. In other embodiments, nitinol has been cured to a certain shape with 2-3 curvature points with radius ranging from 11-17 mm while in others it is cured in a circle of diameter of 4 cm. The curvatures allow the nitinol to be deployed in a predefined trajectory to capture and fragment the nucleus.

The cutting element naturally deploys in a direction straight from the tip. One end of the loops are pushed forward linearly by the actuator, deploying the loops at an angle to each side of the cataract as they pass through the angled holes on the tip. The cutting element has a slight angulation produced by the angled holes on the tip from where it is deployed. The loops do not contain any tension when deployed without obstruction. Tension in the form of torque in the material builds up as the obstruction (e.g., cataract) twists the loop away from its natural course, thereby creating that inward force on the obstruction itself. This tension is maintained by the bridge. As one edge of the loop is pushed away, the tension between the loops that is used to keep them aligned to a pre-defined distance, is maintained by the inelastic material of the bridge.

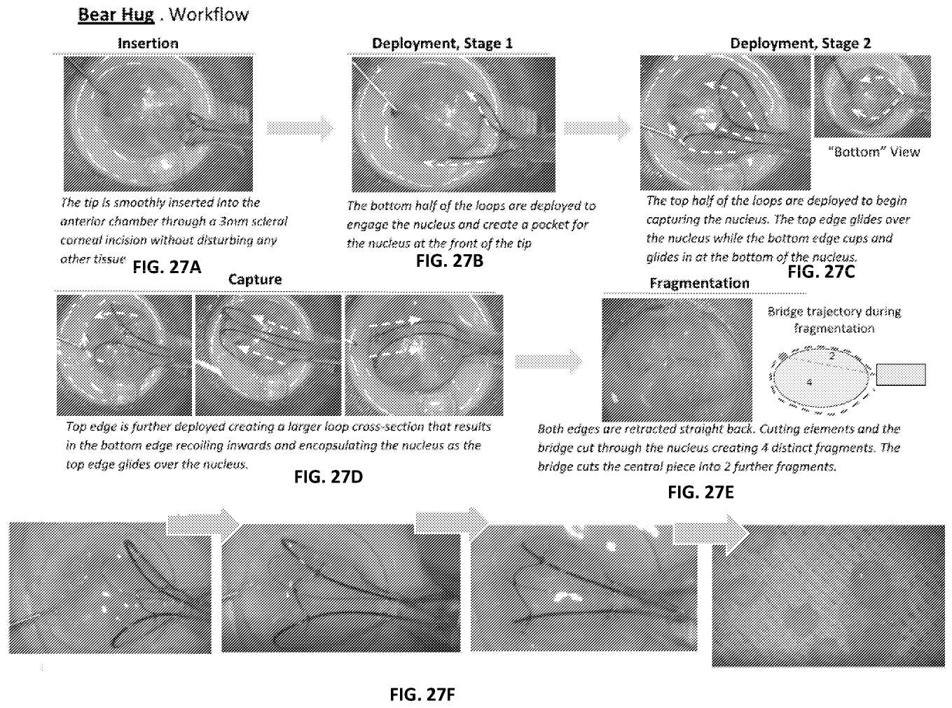

FIGS. 27A-27E illustrate a workflow for using the device designed to hug and fragment the nucleus, according to an embodiment of the present invention. FIG. 27A illustrates the insertion of the tip of the bear-hug device into the AC. The tip is smoothly inserted into the AC through a 3 mm sclera-corneal incision without disturbing any other tissue of the eye. FIG. 27B illustrates a first stage of deployment. The bottom half of the loops is deployed to engage the nucleus and create a pocket to engage and align the nucleus at the front of the tip in preparation for the second stage of deployment.

FIG. 27C illustrates two views of the second stage of deployment. The top half of the loops, with the bridge attached, is deployed to begin capture of the nucleus. The top edges of the loops and glide over the nucleus with the bridge, while the bottom edges of the loops cup and glide laterally inwards towards the cataract centerline at the bottom of the nucleus. The two loops are preferably deployed at angles of 20 degrees to 30 degrees from each other and glide to the side and top-side of the nucleus in a symmetric and synchronous fashion. However, any angle from approximately 0 degrees to 60 degrees could also be used. FIG. 27D illustrates views of the capture of the nucleus. The top loop is further deployed creating a larger loop cross-section that results in the bottom loop recoiling inwards following along the ellipsoid curvature of the surface of the nucleus and encapsulating the nucleus as the top loop glides over the nucleus. For capture, the cutting element moves forward in a unidirectional manner and encapsulates the nucleus with a slight further deployment of the top loop past the equator of the nucleus. In order to ensure the loops are in position to capture, the cross-sectional profile can be adjusted based on how far each loop is deployed. Both loops are then retracted to secure the nucleus. During capture, the bridge maintains tension between the loops forcing the bottom loop to move in and capture the nucleus. The bridge keeps unidirectionality of deployment, while capturing the nucleus. The bridge also conforms to the contour of the nucleus. This conformation to the curve of the nucleus allows for a good grip on the nucleus, preventing it from easily escaping/slipping out through either side of the loops.

FIG. 27E illustrates views of fragmentation of the nucleus. Both loops are retracted straight back. Symmetrically and unidirectionally, the loops and the bridge cut through the nucleus creating four distinct fragments. The loops, moving symmetrically and synchronously divide the nucleus into three fragments, and the bridge cuts through the central fragment to create two fragments, or four in total. In some embodiments, the three loops utilize two bridges with each bridge connecting to two loops, as shown in FIG. 27F. The two bridges can consist of varying lengths with one being shorter than the other. This will reduce the distance between the two loops connected with the shorter bridge while allowing a greater space between the two loops connected to the other bridge. This creates a difference in the space available to capture the cataract while the bottom of the loops are deployed before the deployment of the bridge. This allows the cataract to be encapsulated by the vacant space created between loops at the side connected to the central loop with the longer bridge.

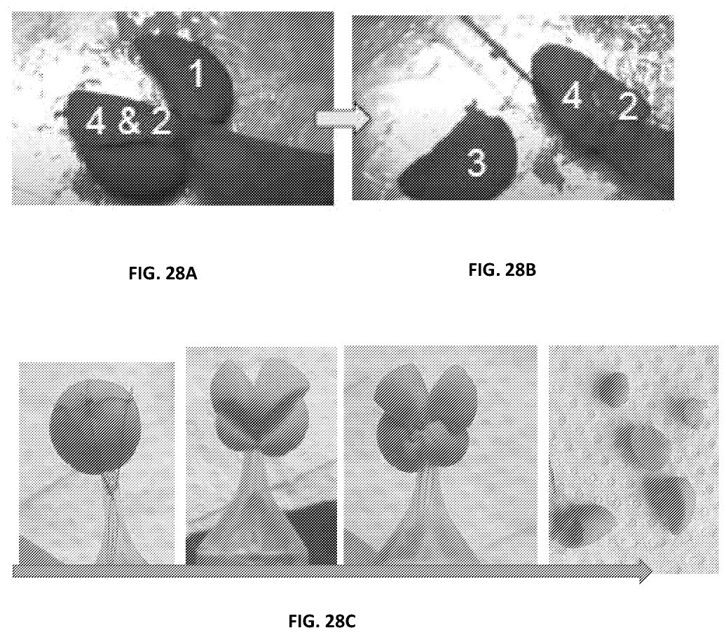

FIGS. 28A and 28B illustrate schematic views of nucleus fragmentation that result from using the device designed to hug the nucleus, according to an embodiment of the present invention. As the cutting element and the bridge are retracted, the nucleus is fragmented into three pieces, and the central fragment of those three is further cut in half by the bridge. This creates four fragments overall. Both loops are retracted together to cut through the nucleus as they move back. The bridge is retracted back, along with the loops and cuts the center fragment while it is also being retracted. The cuts through the nucleus could be straight or angled forming an inward or outward facing slant on the cataract from the top-down view, with variable angles from sixty degrees to negative sixty degrees relative to the neutral straight position. The shape depends on the shape and size of the cataract. The cutting element and the bridge are formed from materials that generate smooth and clean cuts. The bridge keeps the cutting elements in place before and during retraction. The bridge maintains the distance of the top loop and ensures that the cutting elements retract in a straight fashion towards the tip.

While the fragments are created as the cutting elements move through the cataract, a part of the central fragment, directly ahead of the lumen of the tip, is captured within the lumen of the tip and removed upon removal of the device from the anterior chamber of the eye. The portion of the fragment is pushed into the lumen of the tip with the retractive, and compressive forces exerted by the loops and the bridge or bridges on the cataract fragment directly ahead of the tip. The volume of the cataract material pushed into the lumen of the tip is dependent on the size, shape and perimeter of the inner lumen of the distal end of the tip as well as the surface dimension at which the cataract is being engaged and its geometrical dimensions. One or more of the fragments may be removed from the AC in this manner. This method is viable for any number of loops and bridges and reduces the number of cataract fragments and volume of cataract material that the surgeon is required to remove from the anterior chamber.

In other embodiments, the bear-hug is combined with a sheath, a base, and a syringe attached to the main body, to inject viscoelastic or saline solution into the AC to maintain intraocular pressure and remove fragments through the lumen of the tip. In some embodiments, there could be more than one bridge connecting two loops while the bridges are spaced apart along the loops. In some embodiments, the scoop provides stability for the cataract at its bottom, preventing it from moving towards the capsular bag and creating a stable plane on which the cataract can be encapsulated and fragmented. This also allows the fragments to maintain a single plane in the anterior to posterior plane. In some embodiments, a syringe is attached to the embodiment via a locking mechanism, such as a luer lock, at the back of the handle or other position on the device. A channel through the embodiment contains a tube that will allow the passing of viscoelastic material or saline solution from the syringe through the body and exiting at the tip, through holes of various shapes and dimensions, into the anterior chamber of the eye. The tubing can be made of various biocompatible and non-toxic materials and a range of outer and inner diameters to vary the pressure and speed of the material pushed through the tube. The associated channel can vary in diameter depending on size of the tube. The viscoelastic or saline solution can be injected when the loops are being deployed and retracted as required by the user. When retracted, the syringe will pull cataract fragments into the lumen of the device and collect them.

In other embodiments, the device can be deployed and retracted to grip the nucleus and stabilize the position of the cataract in the anterior chamber while another device such as a sinskey hook can be used to break apart the nucleus into the desired number and dimensions of fragments desired. Alternatively, the surgeon may retract the instrument partially, rotate the nucleus and retract the loops again to create multiple intersecting cuts. In other embodiments, the bridge can be made of elastic material that stretches in order to capture the nucleus while the cutting elements move medially in relation to the cataract. In other embodiments, the bridge can be made of metal such as stainless steel or be made in a fashion similar to the characteristics and properties of a spring. In other embodiments, the bridge can be moved up and down the loops via a slider that is connected to the bridge and therefore will not be fixed to a single location on the loops.

In other embodiments, all ends of the cutting elements translate simultaneously along the body as they are all connected to a single slider instead of two sliders. In other embodiments, the top end of the loops is threaded through individual holes at the top of the tip, however these ends are fixed and are not connected to a slider nor are they allowed to move. The bottom ends of the loops are then threaded through the holes of the tip and connected to the slider residing in the body that translates the loops into and out of the tip. This embodiment and configuration prevent the top of the loops from curving upwards and hitting the corneal endothelium by creating an almost linear shape at the top of the loop when looked at from its side. In some embodiments, as shown in FIG. 26I, small diameter tubes are placed within the central lumen of the tip and enter from the bottom of the tip and exit out through the holes on the tip. Each loop end runs through one of these channels with some channels holding two ends of loops when three loops are used. These tubes reduce the accumulation of viscoelastic or other matter on the loops when they are used in the anterior chamber, resulting in a smoother deployment with better overall haptic feedback and prevention of viscoelastic or other foreign matter from getting into the tip or into the body through the tip.

In some embodiments, a retractable side scoop is formed from a steel wire (4-0) that can support the nucleus and scoop it to the correct location. This reduces the cross-sectional profile of the elements in the anterior chamber and is only deployed when needed. The scoop can also be used to collect and remove cataract fragments from the AC. In some embodiments the sliders are placed close together so that both the sliders can be actuated at the same time with the use of the index finger or the thumb. The embodiment may also be used sideways with the cutting elements being assembled in a configuration that can capture the nucleus by using the body sideways. In some embodiments, the sliders are placed above and below the body to make the deployment of the ends of the loops more intuitive. The top slider deploys the top ends of the loops while the bottom slider deploys the bottom ends.

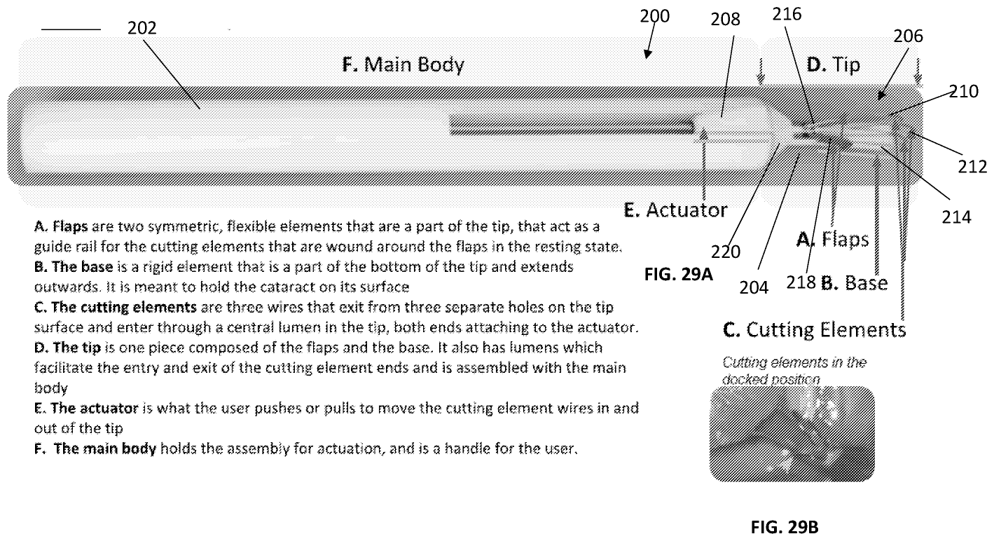

FIGS. 29A and 29B illustrate views of a device with a bi-flap design, according to an embodiment of the present invention. FIG. 29A illustrates a perspective view of the bi-flap device, and FIG. 29B illustrates a perspective view of the cutting elements in the docked position around the flaps, adjacent to a nucleus. The bi-flap device 200 includes a handle 202, a tip 204, and a cutting element 206. The handle 202 houses the actuator 208 and internally houses the mechanisms and tracks for deploying the cutting element 206. The actuator 208 provides adequate grip for the surgeon and carries out linear translation of each end of the cutting element. The actuator can provide haptic feedback through friction between the slider and main body. Additionally, the actuator can lock the cutting element position in place when not in use. The actuator 208 actuates the motion of loops 210, 212, and 214 of the cutting element 206. The actuator 208 can also include texture on its surface to create a grip for the user.

Further with respect to FIG. 29A, the tip 204 houses the cutting element 206 and is the site of deployment of the cutting element 206. The tip 204 is also responsible for the angle of deployment of the cutting element 206 from the tip 204 via distinct, angulated entry and exit channels within the tip or entry and exit points on the surface of the tip. The tip 204 of the bi-flap device 200 includes flaps 216, 218. The flaps 216, 218 are two symmetric, flexible elements that are a part of the tip. The flaps 216, 218 act as a guide rail for the cutting element 206. The loops 210, 212, and 214 of the cutting element 206 are wound around the flaps 216, 218 in the resting state. The flaps 216, 218 keep the cutting element 206 docked at an angle to the midline running through the center of the device dividing the flaps to allow for initial separation and deployment of loops 210, 212, 214 along the sides of the cataract. Once the flaps engage with the surface of the nucleus, the flaps will be pushed to either side of the cataract, enabling the loops to move to the side of the cataract. The flaps 216, 218 provide visibility on engagement of the nucleus with the flaps and on cutting element motion.

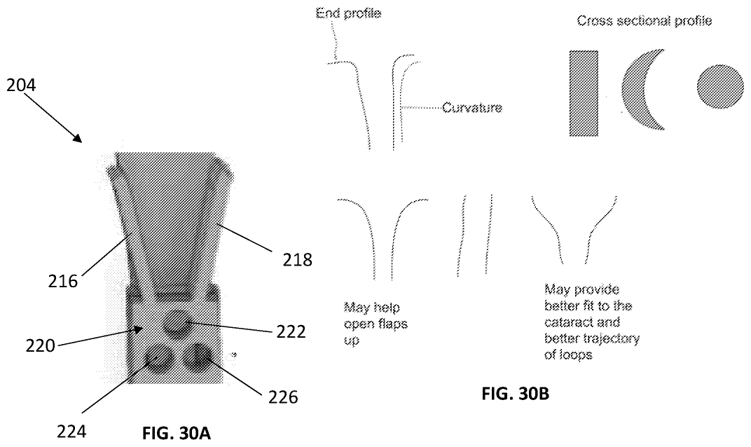

Holes, illustrated further in FIG. 30A are positioned symmetrical to each other around the mid-plane with diameters between approximately 0.05 to approximately 0.6 mm. Placement of flaps 216, 218 on the tip 204 is relative to the placement of the hole. Each cutting element exits through its individual hole and may also enter through its individual hole/channel through the tip or through the base or both. In one embodiment, holes are placed in a triangular configuration relative to one another on the center of the tip, while flaps are placed on either side from the center, positioning the cutting element loops to the sides when they are in their docked position. In other embodiments, two holes can be placed on one side and one on the other or all three holes can be placed linearly. Holes can be placed in any configuration to achieve a range of loop angles and trajectories.

Additionally, with respect to FIG. 29A, looping the cutting elements around the flaps enables transient interlocking of the cutting elements with the flaps. The flaps 216, 218 can be different colors and can have a reflective surface to increase visibility. Generally, the flaps 216, 218 are formed from a flexible material including plastic, such as polytetrafluoroethylene and polyamides, polyimides, or flexible shape memory alloys, such as nitinol, so it can bend inward when there is a force on the outer surface of the flaps and outward when there is a force applied at the tips of the internal surface of the flaps. Flap flexibility provides a snug fit with the cataract and accommodation to a variety of cataract diameters and surface curvatures. The force applied by the cutting element on the flap is less than the force required to bend the flap and the force by the cataract that is keeping the flaps open. Flaps are able to apply enough counteracting force to prevent deflection of the bent cutting elements that are looped around the flaps back to the center and maintain loop position to the sides of the cataract after the flaps have engaged with the cataract. In other embodiments, the flaps may be in the form of a funnel with holes, where the angle of the funnel determines the trajectory of the loops which exit and enter from holes in the funnel.

Further with respect to FIG. 29A, the base 220 of the tip 204 is a rigid element of a bottom part of the tip 204. The base 220 extends outward towards the handle 202 and is designed to hold the cataract from the bottom and stabilize its movement within the AC. The base 220 prevents movement posteriorly and tilting of the nucleus. The base 220 helps to position and center the cataract with respect to the flaps. The base 220 has adequate width to be able to support the cataract without tilt. The width of the base is less than approximately 3 mm with a maximum length of approximately 10 mm. The base can be either in the form of a complete surface, or in the form of two parallel lines/prongs or in the form of a loop. The base can be stationary or deployable and retractable in conjunction with or separate from the cutting elements. The base can be colored and reflective to be visible underneath the cataract and is centered to the cutting elements. In some embodiments, the base may also contain holes through which the cutting elements can pass through, helping to further maintain the tension of the cutting elements as they are deployed to capture the nucleus and maintain the position of the cutting elements during retraction. The tip 204 also defines lumens, which facilitate the entry and exit of the cutting element 206. The lumens occur on at least the top surface of the tip but may also be present on the bottom surface of the tip to create individual entry and exit points for each cutting element. The tip 204 including the base 220 and flaps 216, 218 can be formed as one piece, but from different materials for each part or as an assembly of parts. The tip 204 can be formed from a rigid material such as stainless steel, rigid polypropylene, or other biocompatible metals, polymers and elastomers. Tip cross-sectional profile can be square, rectangular, circular, elliptical, or any other irregular but symmetric shape.

As illustrated in FIG. 29A, the cutting element 206 includes the three loops 210, 212, and 214. The loops 210, 212, and 214 exit from three separate holes defined by the tip and enter through a central lumen defined by the tip. Both ends of each of the loops 210, 212, 214 of the cutting element 206 are coupled to the actuator 208. The loops 210, 212, 214 are positioned equidistantly from each other and spanning the top and bottom surface of the cataract. The cutting element 206 encloses the cataract and is tensioned to fragment the cataract. The middle loop 212 of the cutting element can be greater in length than the side loops 210, 214 of the cutting element 206. The cutting element 206 can be made from a variety of materials including shape-memory materials such as nitinol. For uncured shape memory or non-shape memory elements, height is determined by distance between top and bottom holes and loop curvature height can be constrained by curing the nitinol elements.

FIG. 30A illustrates a top-down view of the bi-flap portion of the tip, according to the present invention. FIG. 30A shows the tip 204 which includes flaps 216, 218 and base 220. FIG. 30A also illustrates the placement of holes 222, 224, and 226. The holes 222, 224, 226 direct cutting element direction. The holes 222, 224, 226 determine loop height of the cutting element, angulation of the cutting element while being deployed, and its final position along the cataract once it encompasses the cataract. Holes 222, 224, 226 determine cutting element loop spacing relative to each other and maintain the position of cutting elements during retraction. Holes 222, 224, 226 align the top and bottom portion of all loops and enable cutting elements to form closed loops. Holes 222, 224, 226 keep the elements separated. Holes 222, 224, 226 are positioned approximately 1 mm-3 mm below the position of the base (starting from the most proximal portion of the base, such that the assembly including the holes fits within an 11 mm diameter anterior chamber. The holes can be spaced 0.1-0.7 mm apart (on the same plane) and in a variety of configurations to subtly alter loop spacing/final position. All top lumens or holes are in the same plane (in the anterior-posterior direction). In the case that there are separate lumens at the bottom, all of these holes are also in the same plane. The lumen through which the cutting elements enter the tip is a few planes posterior to the holes through which the cutting elements exit the tip (cutting element entry and exit points are on different planes and angles (in the anterior-posterior direction). Cutting elements enter the bottom central lumen perpendicular to the lumen cross section while the exit points at the top are angled in two planes—oriented in the lateral plane to either side of the cataract (left or right depending on the flap side).

FIG. 30B illustrates the various configurations of the flap profile, including the end profile, angle, and cross-sectional profile. The tip cross-sectional profile can be square/rectangular, circular or elliptical, with a width between approximately 2-2.75 mm. Holes are positioned “internally” to the position of the flaps.

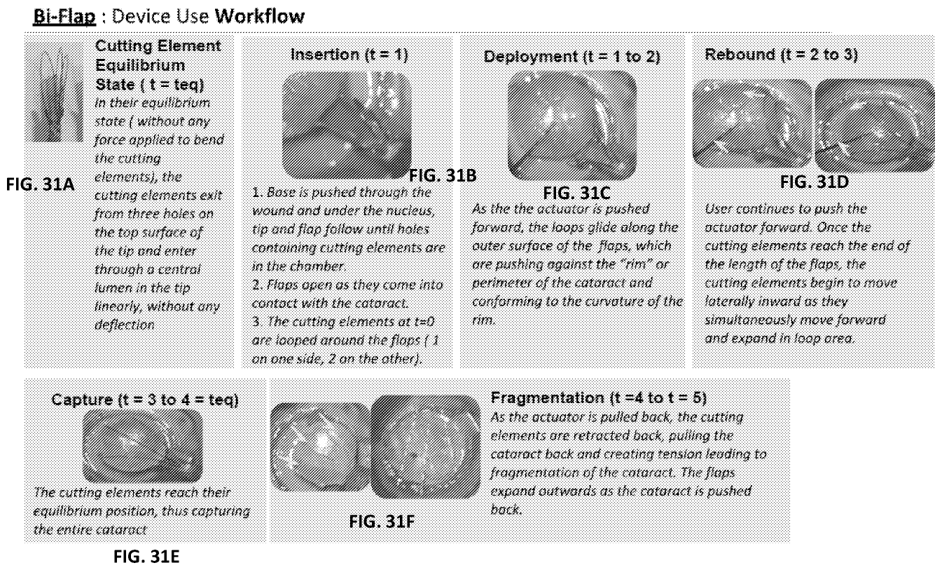

FIGS. 31A-31F illustrate a workflow for using the bi-flap device, according to an embodiment of the present invention. FIG. 31A illustrates a cutting element in equilibrium state. In their equilibrium state (without any force applied to bend the cutting element loops), the loops exit from three holes on the top surface of the tip and enter through a central lumen (or through individual lumens that connect to a central lumen in other embodiments) in the tip linearly, without any deflection. FIG. 31B illustrates insertion. The tip is pushed through the incision and under the nucleus. The flaps and the base follow until the holes containing the loops are in the AC. When inserting the tip into the incision the flaps are pushed laterally inward to allow for easy entry. The flaps open outward as they come into contact with the nucleus. The loops are looped around the flaps (one on one side and two on the other). FIG. 31C illustrates deployment. As the actuator is pushed forward, the loops glide along the outer surface of the flaps, which are pushing against the rim or perimeter of the cataract and conforming to the curvature of the rim. FIG. 31D illustrates rebound. The user continues to push the actuator forward. Once the loops reach the end of the length of the flaps, the loops begin to move laterally inward as they simultaneously move forward and expand in loop area. FIG. 31E illustrates capture. The loops reach their equilibrium state, as illustrated in FIG. 31A, thus capturing the entire cataract. FIG. 31F illustrates fragmentation. As the actuator is pulled back, the cutting elements are retracted back, pulling the cataract back and creating tension leading to fragmentation of the cataract. The flaps expand laterally outwards as the cataract is pushed back.

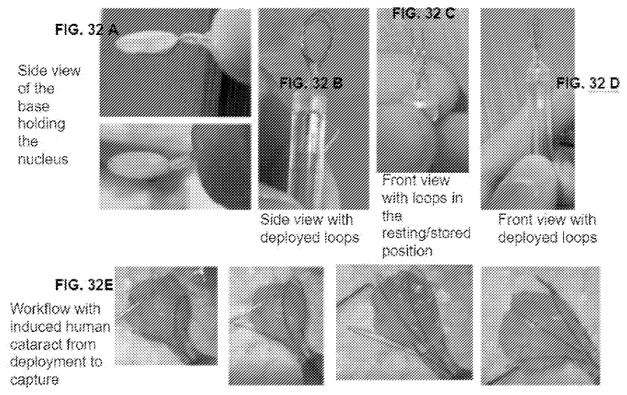

FIGS. 32A-32D illustrate views of an embodiment where the base acts as a guide rail or flaps for the loops, without having separate flap elements. This embodiment further reduces the space that the device is occupying in the eye while maintaining the controlled side movement of the loops. FIGS. 32A-32C show how the base can have a curvature that conforms to the posterior surface curvature of the nucleus. This curvature allows the nucleus to come closer towards the wound and engage with the device upon lightly pressing the posterior lip of the wound while entering with the tip. FIG. 32B shows a side-view of the base and the deployed loops, while FIG. 32C shows a top-down view of the base and tip with the loops in the stored or resting position, being looped around the base elements. FIG. 32D shows the top-down view of the embodiment with loops fully deployed. The base which is acting as the flaps or guiderail performs similar to the workflow in FIG. 31, as shown in FIG. 32E.

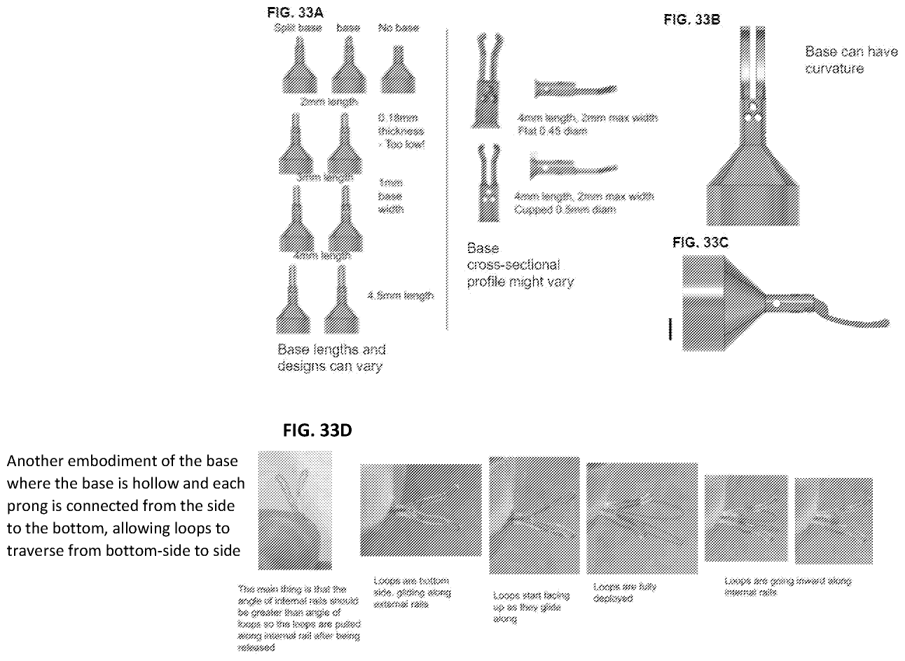

FIGS. 33A-33D illustrate views of various configurations of the base, according to the embodiment of the invention illustrated in FIG. 32. The base can be composed of two or more “prongs” or can be closed to form a loop, as shown in FIG. 33A. The surface of the base can be flat, cupped or rounded, as illustrated in FIGS. 33B and 33C. Modifying the curvature of the base can allow for modification of the point at which the cutting elements begin to move inward towards the centerline of the cataract. The angle of the prongs can be parallel, away from each other, or towards each other in order to modify the cutting element trajectory accordingly. In some embodiments, such as the one shown in FIG. 33D, the prongs can be wire frames that are angled. When the angle of the wire frame, especially towards the end is greater than the angle of either of the side loops, the loops will traverse along the wire frame, first along the external wire, and then along the internal wire. This will also ensure a consistent retraction trajectory of the cutting elements. In some embodiments the prongs can have alternative curvatures, resulting in a variety of three dimensional shapes with a range of radius of curvature.

The base can be composed of a variety of materials including stainless steel, other types of biocompatible metals, or rigid plastics. The base can be coated or covered with a softer material such as silicone. The end of the base can be bent inwards and/or blunt. The base may have small ports for irrigation. The base length is preferred to be 6 mm but can be anywhere between approximately 3 mm to approximately 10 mm. The base can also be used to remove one or more of the fragments that are created with retraction of the cutting elements. The base can be stationary or retractable. In other embodiments, the bottom portion of the cutting element may be connected to the base with a connecting element that can be moved across the rail. This would allow the cutting element to expand and capture the nucleus, while preventing cutting element slippage and maintaining a straight cutting element trajectory upon retraction.

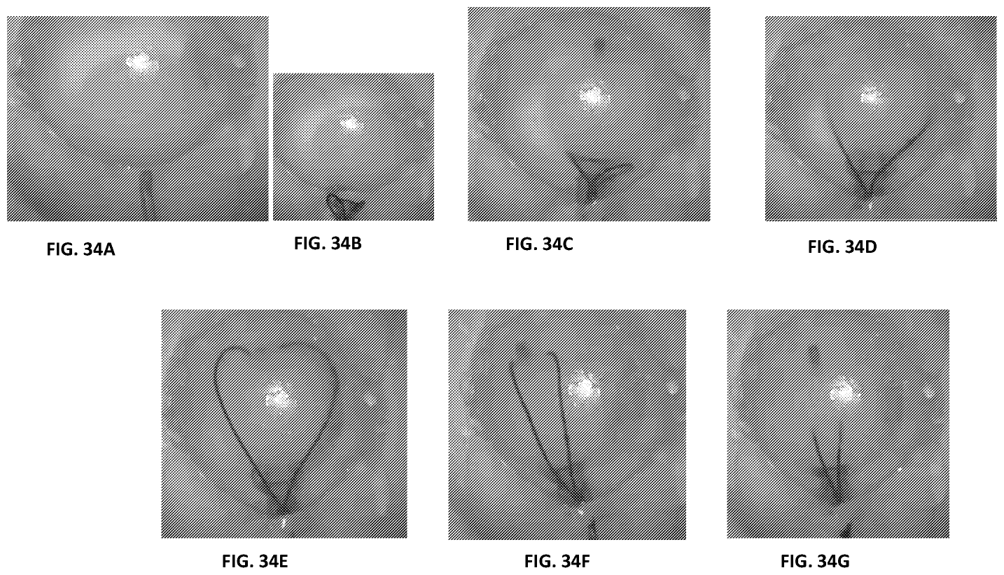

FIGS. 34A-34G illustrate a workflow for using an embodiment of the present invention to create a three-piece fragmentation. As illustrated in FIGS. 34A and 34C, the tip is inserted within a 3 mm corneal SC incision. The end of the base enters below the tip followed by the rest of the base including the flail and finally the funnel and holes. A surgeon needs to ensure that the base is the same plane of the iris—flat and flushed with the bottom of the nucleus. The funnel may be rotated 90 degrees to the left or right for the insertion of the funnel into the tunnel of the incision. As illustrated in FIG. 34C, the edge of the base is positioned to the furthest end of the nucleus. This should be visually clear. The loops are deployed slowly while maintaining contact with the cataract and the funnel. The loops glide along the side of the nucleus—the middle of the loops will engage the middle of the nucleus. As the loops deploy, they are moved to the side by the flail on the bottom of the base. As illustrated in FIG. 34D, the loops are deployed further. The loops encircle the nucleus as it moves along the side of the nucleus. The top edge of the loop moves over the top surface of the nucleus. Loops are translated straight. As illustrated in FIG. 34D, the tops of the loops move above the circumference of the nucleus and they glide along the top surface of the nucleus. As illustrated in FIG. 34F, the actuator is slowly retracted to pull the loops taut to position the loops fully in contact with the nucleus and further straighten the loops. The loops are maneuvered to the ideal location to fragment the nucleus in pieces. As illustrated in FIG. 34G, the loops are retracted along with the bridge that moves within the vacant space between the top and bottom bridges. The bridge keeps the translation of the loops steady, and consequently the retraction of the loops cuts the nucleus to make 3 nucleus fragments. It should be noted that three fragments are created with the two loops and a lower bridge, and four fragments are created with two loops and a diagonal bridge—the fourth fragment is created by cutting the central fragment into two pieces.

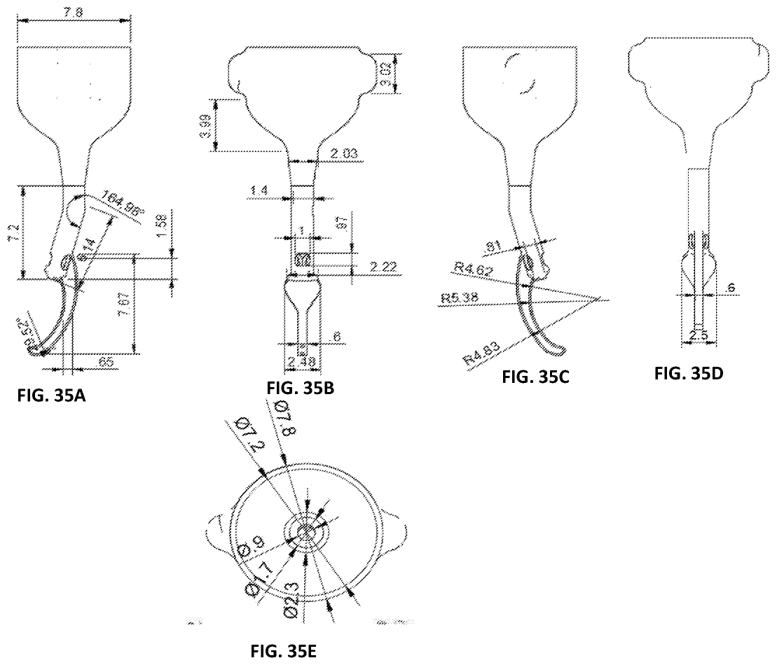

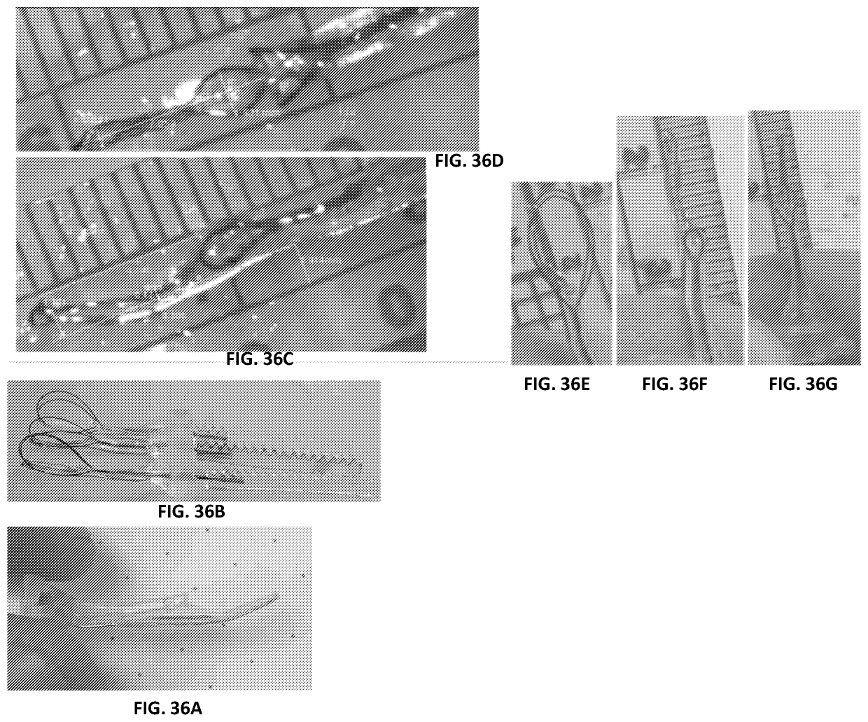

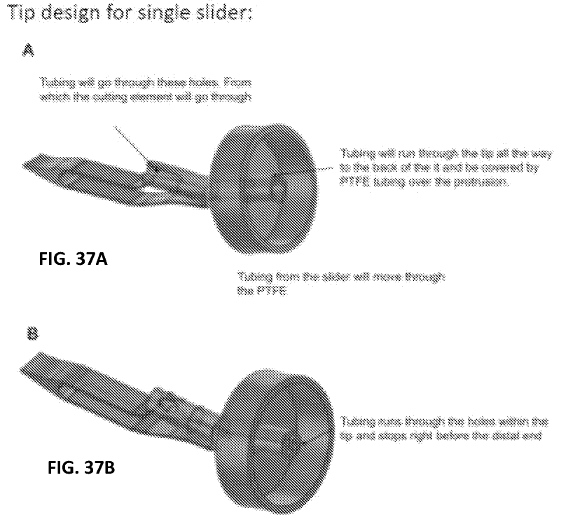

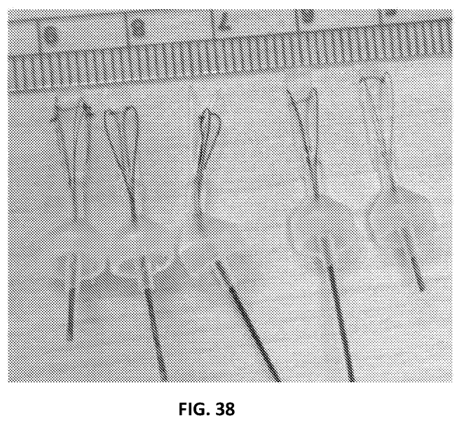

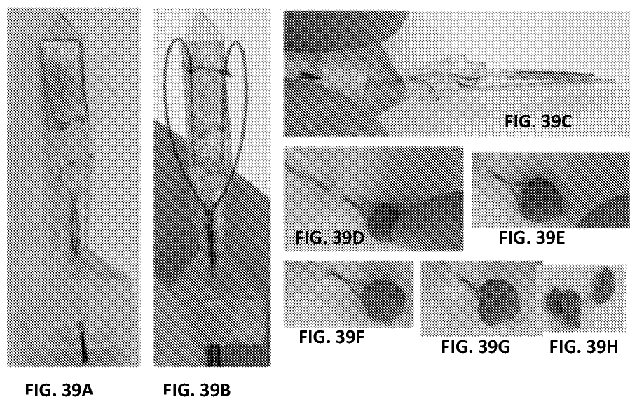

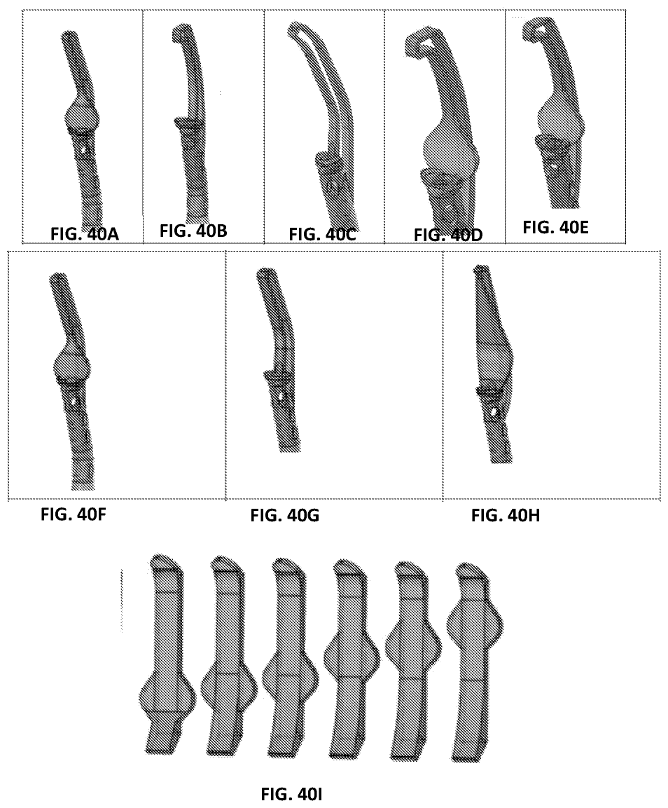

FIGS. 35A-35E illustrate views of a tip and a base, according to an embodiment of the present invention. FIGS. 36A-36G illustrate views of a tip, a base, and a cutting element, according to an embodiment of the present invention. FIGS. 37A and 37B illustrate a tip design for use with a single slider, according to an embodiment of the present invention. FIG. 38 illustrates views of various implementations of the tip design of FIGS. 37A and 37B. FIGS. 39A-39H illustrate views of the tip embodiments of FIGS. 37A and 37B. FIGS. 40A-40I illustrate views of the tip embodiments of FIGS. 37A and 37B.

The tip includes a circular lumen. In some embodiments, the outer diameter is 1.5 mm and an inner diameter is 1 mm. In some embodiments, the top hole is a 1 mm by 1 mm square that allows for the top end of both the loops to go through. The top hole is 1 mm to 3 mm from the edge of the tip. Two bottom elliptical holes that are 0 mm to 3 mm apart (mid-point to mid-point). A hole length, in an exemplary embodiment, is from 0.2 mm to 1.6 mm and width is 0.2 mm to 1.6 mm. The holes are positioned symmetrically across from one another. Holes are extruded 90 degrees to the plane of the surface of the tip. The holes taper from at least 2 mm from the edge of the tip. In some embodiments, the tip is angled between 0 to 90 degrees, and is positioned between 2 mm to 10 mm from the edge with a radius of curvature from 0 mm to 10 mm.

The funnel defines an elliptical shape at one end or both ends. A distal end of the funnel is attached to a proximal end of the tip. The end of the funnel attached to the tip conforms to the geometry of the tip, where the connection is made, while the exposed end varies in shape and geometry and length. (elliptical, circular or polygonal, with length from 0.2 mm to 3 mm and lengths from 0 mm to 3 mm. The funnel and its shape enables the loops to deploy to the side of the cataract. Before deploying the loops, the widest edge/circumference of the cataract will be positioned flush with the funnel and supported by the base.

A top side of the base is attached to the proximal end of the funnel, and a bottom side of the base is attached to the back of the tip and below the furthest edge of the bottom holes. The based provides a scaffold for the bridge at the bottom. The base also has a curvature to enable smooth translation through the vacant space between the top and bottom bases of 0.1 mm to 0.5 mm—width is equivalent to the diameter of the nylon monofilament sutures.

A flail at the beginning of the base pushes the loops to the side, at an angle to the direction of translation, between 0 mm to 5 mm from the edge of the funnel to create space between the loops and capture the nucleus by allowing the top of the loops to glide along the periphery of the nucleus, thereby allowing the loops to capture the nucleus once the loop is fully deployed. The flail provides a scaffold for the nucleus to sit on top of, and opens-up the wound during insertion. The flail also creates a space between the loops to enable capture of the nucleus.

An angle at the end of the base enables the bridge to be on the same plane as the widest circumference of the cataract, or close to it. This enables the top of the loops to move into the cataract, due to the motion of the bridge pulling the bottom of the loops up and therefore pulling the top of the loops along with it. The tops of the loops glide along the bottom of the cataract and pass the widest circumference, and once past that they move into the nucleus with a width defined by the bridge.

A space between the top and bottom base allows for the bridge to glide smoothly between the top and bottom sides of the base for the deployment and retraction of the loops. The space extends throughout the entire base.

The cutting element is formed from 4-0 nylon sutures with a circular diameter. The cutting element in some embodiments takes the form of a pair of loops that in turn are formed from wires. The cutting element is configured to cut through all grades of cataract. The wires have a high flexural modulus to conform to the cross-sectional areas of different cataracts. Each end of the wires is threaded through a single hole on the tip. Rotating the tip rotates the loops. Both loops are symmetrical and deployed together. Both loops have the same cross-sectional area (elliptical with 11 mm length and 4 mm height) In some embodiments, the loop closer to the cataract has a larger cross-sectional area. The cutting element, responds to actuation by the surgeons as one whole element—deploying and retracing as a unit together with the bridge

The bridge loop junction is made of a single knot. Any knot known to or conceivable to one of skill in the art and suitable for the intended purposed can be used. The bridge loop junction is able to withstand cutting forces while the loops and bridge are cutting through even the most mature cataract. The bridge loop junction is immobile. In some embodiments, the bridge loop junction is located symmetrically across from the other loop, while in other embodiments, the bridge loop junction is located diagonally across the loops of the cutting element.

The bridge is made of monofilament nylon 6-0 sutures. The bridge can be a variable length from 3 mm to 5 mm. The bridge can be placed in a number of locations. It can be placed symmetrically along the loop, symmetrically on both loops, such that it does not participate in cutting the cataract, or diagonally, such that it participates in fragmenting the central portion of the cataract. Alternately, the bridge can be adhered to the cutting element, such that it is immobile. If knots are used to connect the bridge to the loops, the knots can be spherical and from a diameter of 0 mm (does not protrude out) to 2 mm. Alternately, any form of knot known to or conceivable to one of skill in the art can also be used.

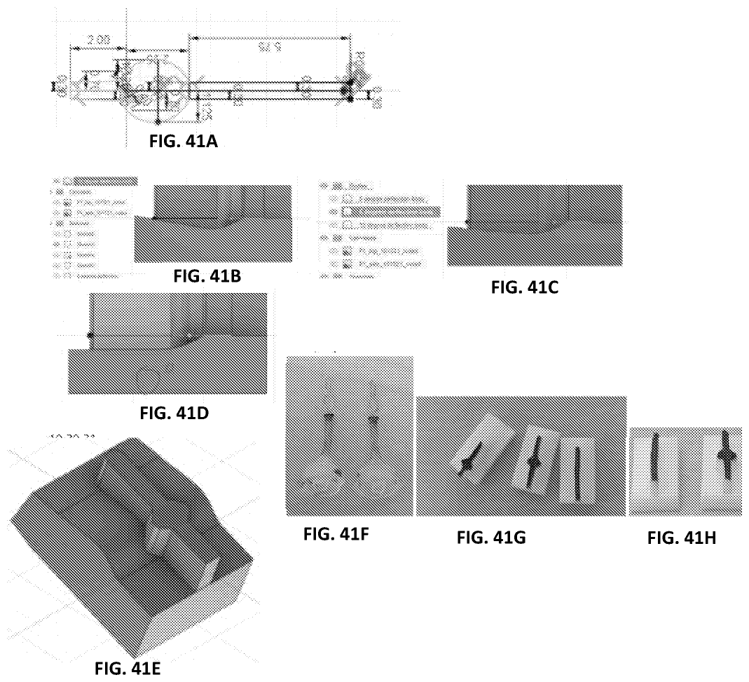

FIGS. 41A-41H illustrate manufacturing tools for manufacturing components of a device according to an embodiment of the present invention. FIGS. 41B-41E, in particular, illustrate molds and fixtures for forming a steel wire into the preferable flail geometry. The molds can be generated in any way known to or conceivable by one of skill in the art.

The knots connecting the bridge to the loops are made in some embodiments by spreading a bonding agent such as in some cases Loctite on the surface and dropping a small volume of cyanoacrylate (CA) on the knot to fill in any gaps between the knot created by the 6-0 suture on the 4-0 suture in that order. Any medical grade CA can be used.

In use, the method of the present invention includes several stages. Stage 1 includes the insertion of the device through the wound. The tip is inserted into the anterior chamber of the eye through a 3 mm incision. The tong of the device is placed under the nucleus while it is inserted into the anterior chamber. The base element and the tip can also be turned 90 degrees to be inserted into the nucleus.

In stage 1, the cutting element is comfortably docked within the tip allowing for smooth entry into the wound. In some embodiments, a nylon polymer is used to form the cutting element. The nylon polymer of the cutting element does not deform when bent. The nylon surface is smooth (surface roughness characterization) and enables smooth deployment and retraction. Nylon conforms to the shape of the lumen it is forced into within the tip. Nylon sutures, such as the material that forms the main loops, have a circular shape. Nylon (0.15 mm-0.05 mm diameter) used in the present invention has a cross-section making it easy to deploy and store.

In stage 1, the flail on the base participates in opening up the wound for the funnel to enter the loop. The flail supports the nucleus once it enters the anterior chamber of the eye. The tong on the base is inserted first and under the nucleus to create a scaffold to prevent the nucleus from falling below the base and provide further support to the nucleus. The tong placed under the nucleus provides the surgeons with a clear potential path for which the bridge will move along and therefore move the loops from either side of the bridge.

In stage 1, the bridge is docked at the bottom edge of the tip (for straight bridges) or at a diagonal at the end of the tip (for diagonal bridges). In some embodiments, a diameter of 0.05 mm creates a small cross-sectional profile. A smooth polymer material, such as nylon or PP, allows for easy docking. The bridge can be flush with the length of curvature between the 2 holes at the bottom of the tip. In some embodiments, the diagonal bridge is flush with the edge of the tip. The attachment points of the bridge are docked at the periphery of the elliptical holes at the tip.

In stage 1, the holes are placed 0.5 mm-1 mm away from the edge of the tip to prevent the loops from getting stuck within the scleral-corneal tunnel of the anterior chamber. If the loops get stuck they could damage the scleral-corneal tunnel and be unable to deploy well. A length of the tip is between 1 mm to 40 mm to limit the length that goes into the anterior chamber for added safety. In a preferred embodiment the length of the tip is 7.2 mm. This enables the surgeon to focus on deploying and manipulating the loops to capture the cataract. Tapering of the tip between 2 mm to 40 mm away from the edge of the tip plugs the wound and prevents pressure loss while the device is used within the anterior chamber. In a preferred embodiment, the tip is tapered 6 mm after the edge. The tip fits through a 2.75 mm corneal and sclera-corneal incision. An inner diameter of the tip is 1.2 mm, while an outer diameter is 1.5 mm.

In Stage 2, the funnel is positioned to a front edge of the nucleus. The funnel is therefore in the anterior chamber of the eye and close to the wound. In stage 2, the cutting element is docked securely within the holes of the tip. The cutting element does not move before the actuator is engaged to deploy the loops. The flail on the base participates in opening up the wound for the funnel to enter the loop. The tong on the base was inserted first and under the nucleus to create a scaffold to prevent the nucleus from falling below the base/support the nucleus. The tong placed under the nucleus provides the surgeons with a clear potential path for which the bridge will move along and therefore move the loops from either side of the bridge. The width of the flail can be from 1 mm to 5 mm in width—in a preferred embodiment it is 3 mm in width. The width of the top of the base is at least 0.5 mm with a thickness of at least 0.01 mm. The top of the base in contact with the nucleus has a low surface roughness. The tong can have a Small cross-sectional profile (at least 0.1 mm in diameter—overall cross-section with the top and bottom bases). The material of the tong can have a stiffness similar to Silastic™ material. The stiffness of the material prevents the top and bottom of the base from pinching as the nucleus sits on the top part of the base. The base can have a gradual curvature of 4 mm radius to 6 mm radius at different points along the length of the tong.

In stage 2, the bridge is docked at the bottom of the tip—flush with the surface between the two holes at the bottom of the tip. The bridge is docked at the start of the vacant space between the top and bottom elements of the base. The Bridge does not move until it is actuated. Holes placed 1 mm away from the edge of the tip to prevent the loops from getting stuck within the scleral-corneal tunnel of the anterior chamber and therefore damaging it and being unable to deploy well. Length of the tip is between—X cm to (1) limit the length that goes into the anterior chamber for added safety, (2) Enable the surgeon to focus on deploying and manipulating the loops to capture the cataract.

In stage 2, the funnel has variable shape to contact the nucleus—circular/elliptical to pentagonal. The side of the funnel that touches the nucleus is flat or concave. The width of the furthest part of the funnel is configured to allow for good contact with the nucleus (width is 1 mm to 4 mm)

In stage 3, the loops begin to deploy. The loops are deployed to start encircling the nucleus. In stage 2, the cutting element, keeps the nucleus in position, as the loops are being deployed. The motion of the cutting element prevents cataract movement in the lateral directions. Loops create a scaffold on the lateral sides of the cataract to further prevent its motion laterally. The cutting element glides along the side of the nucleus, before translating to the top upon adequate deployment of the loops. The rigidity of loops keeps the cataract in place while the loops are being deployed. The nylon loops of the cutting element maintain their plane and angle of deployment in reference to the holes. The tension created between the bottom holes and the top holes allow the nylon loops to glide along the side of the nucleus at an angle.

In stage 3, the base provides a scaffold to hold the nucleus in position in the vertical and in the forward and backward direction. The base enables unrestricted forward motion of the bridge through the vacant space between the top and bottom base elements. The flail keeps the loops open as they are deployed to enable the loops to glide along the back of the nucleus. In some embodiments, the base has a length of 3 mm to 15 mm. A height of the vacant space is at least 0.01 mm to 4 mm in height across the vacant space. The surface within the vacant space is smooth. In some embodiments the base is the same width as the bridge, with a width between 1 mm to 5 mm. The flail is positioned between 0-3 mm from the edge of the funnel.

In stage 3, the loops are translated between the top and bottom bridges. The bridge ensures smooth translation of the loops. The bridge does not hinder the movement of the loop or the nucleus. Generally, the bridge is formed from a material with high rigidity at short lengths. At short lengths, fewer polymer chains are present per length resulting in less deformation/flexibility of the overall material within a selected length. The bridge is immovable and adhered symmetrically to both loops of the cutting element. The length of the bridge is between 1 mm to 4 mm. The bridge translates well within the vacant space between the top and bottom planes of the base. A length of the bridge is equivalent to the length of the top base.

In stage 3, the tip includes a funnel that enables the loops to move to the side of the nucleus during the initial deployment. Top hole enables the loops to move straight towards the nucleus. A width of the funnel is between 0 mm to 4 mm. A side of the funnel is smooth. The funnel geometry is elliptical with a width between 1 mm to 5 mm and a height of 1 mm to 5 mm. The top hole has variable shape—0 edges to 8 edges and variable size. An exemplary size is 1.5 mm by 1.5 mm.

In stage 4, the tops of the loops enclose the nucleus. As the loops are further deployed, the top edge of the loops enclose the nucleus from the top and move to the center of the nucleus, positioning themselves symmetrically away from the midplane of the cataract and slightly more than the distance between the mid points of the holes at the bottom of the tip. The midpoint of the loops moves along the circumference of the nucleus. A top edge of the loops starts to move from the circumference of the nucleus towards the center of the nucleus by gliding along the top surface of the nucleus

Bottom edge of the loops provide the rigidity to the whole cutting element assembly to ensure a high resolution of feedback between the actuation by the surgeon and the amount of deployment by the loops—1 mm of actuation leads to 1 mm length of loop deployment. Tension between the top and bottom edges of the loops enable the loops to encircle the nucleus to a point. The loops keep symmetrical distance (10 mm to 1 mm) between them at the midline of the nucleus. 1 mm of actuation results in 1 mm length of loop deployment—if loop is at an angle, this might result in less than 1 mm. There is no lag time between actuation and loop deployment

In stage 4, the tong of the base is placed under the nucleus and provides the surgeons with a clear potential path for which the bridge will move along and therefore move the loops from either side of the bridge. An angle at the edge of the base enables the bridge to move closer to the widest diameter of the nucleus, thereby giving the loops a better chance of encircling the cataract. A height of the vacant space is at least 0.01 mm to 4 mm in height across the vacant space. The base has a smooth surface within the vacant space. The base has the same width as the bridge. A height of the angle is at least 0.01 mm to 4 mm. The length of the angle is at least 0.01 mm to 4 mm. The angle is at least 0 degrees to 90 degrees.

In stage 4, the bridge smoothly translates within the vacant space between the top and bottom base. The bridge smoothly moves up the angle at the edge of the base, and the bridge does not deviate or buckle to the sides. The bridge fits within the vacant space, a diameter of the loop is within the height of the nylon bridge. The bridge is rigid enough to maintain the horizontal translation from the front to back. The bridge is the width of the top and bottom bases/tong.

In stage 4, the holes at the bottom and the top of the tip keep tension between the top and bottom edges of the loops. A horizontal distance between the midpoint of the holes at the bottom and that at the top (from 0 mm to 3 mm)—is roughly 0.5 mm with a preferred embodiment.

In stage 5, the loops are positioned at the midline of the cataract. The loops encapsulate the nucleus and are positioned to be symmetrically aligned on the nucleus for fragmentation. Positioned to be symmetrical along the midline of the nucleus. The loops glide along the nucleus to be positioned. When taut, the loops have an increased rigidity that allow them to have minute motioned to be positioned on the nucleus.

In stage 5, the base enables positioning of the loops by sweeping the base to the left or right, thus moving the bridge along with it and in effect moves the loops to position on the nucleus. The base acts as a scaffold to keep the nucleus in position, vertically and in the front-back (top-down) direction. The base prevents the bridge from moving out of the space between the top and bottom base—height of the space is similar to that of the bridge diameter or a slightly more (0.1 mm more). The base maintains its translational position along the base.

In stage 5, the bridge maintains its position within the vacant space of the top and bottom parts of the base. The bridge does not move out of the base. The bridge moves the loops along with it. The diameter of the loops is equivalent to the height of the vacant space. A length of the bridge is equivalent to the width of the top and bottom base. The bridge is immovable and attached symmetrically to the loops.

In stage 6, the nucleus is fragmented. The loops are retracted back to fragment the cataract of any grade. The cutting element is configured to withstand the pull force required for fragmentation of a mature nuclear sclerotic cataract. The loops retract in a linear direction towards the holes on the tip. The cutting element has a knot-pull force of at least 6N—reality 8N on average. The cutting element has a rigid structure and straightening of the wire when a force pulls on it.

In stage 6, the base enables the bridge and loops retract in a linear fashion without deviation in the same angle the tong of the base is facing. The bridge has a smooth retraction within the space between the top and bottom of the base. The bridge moves in the same direction as the tong of the base. The bridge pulls the loops in the same direction as its retraction. The bridge pulls the loops at the same time without delay as it is being pulled.

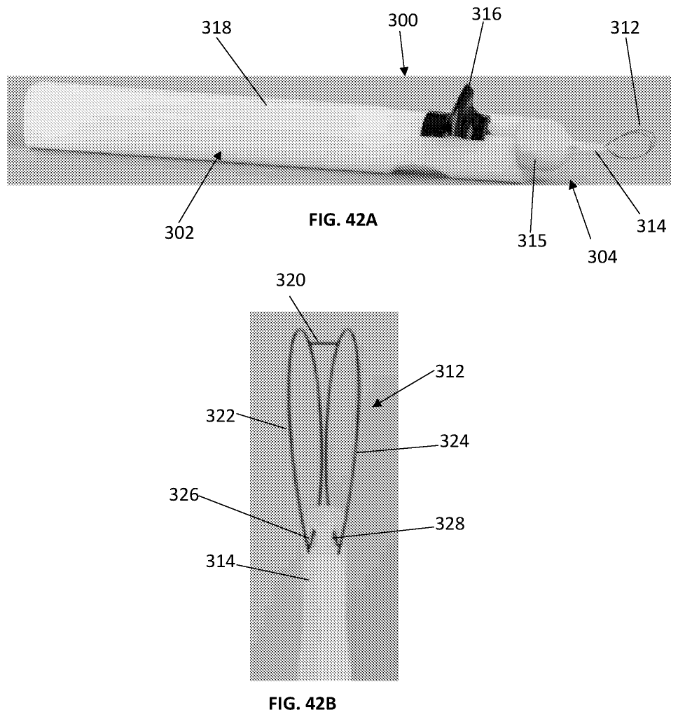

FIGS. 42A and 42B illustrate views of a device for fragmenting a cataract, according to an embodiment of the present invention.

The device 300 includes a body 302 and a tip end 304. The body 302 includes handle 318 and actuation mechanism 316. The tip 304 includes a base 315, a tip 314, and a cutting element 312. The handle 318 internally houses the actuation mechanisms 316 for deploying the cutting element 312, which will be described further herein. A bridge 320 couples the loops 322 and 324 of the cutting element 312. The actuation mechanism 316 provides adequate grip for the surgeon, and carries out linear translation of each end of the cutting element. The loops 322 and 324 are deployed from holes 326 and 328 in the tip 314.

In some embodiments the actuating mechanism can take the form of a slider. The slider includes a sliding mechanism that moves up and down the body to deploy the cutting elements in the direction of actuation. The surgeon will likely either use their index finger or thumb to actuate the slider. In other embodiments, a gear based actuation mechanism is used. The gear-based mechanism includes a rack attached to the cutting elements, a larger gear, a smaller gear and a wheel that moves the smaller gear. The gear-based actuation mechanism provides a mechanical advantage to the system by using a smaller gear with fewer teeth attached to the wheel. Actuated by a wheel mechanism that is rotated by a finger of the surgeon and translates direction of motion of the finger to the direction of motion of the loops (i.e. forward dial=forward deployment of the loops).

The actuation mechanism is configured to provide adequate grip for the surgeon. The actuation mechanism can be held by both left and right handed surgeons with ease. The actuation mechanism allows for linear translation of the cutting element, and provides haptic feedback to the user. Further the actuation mechanism provides mechanical advantage to the user to cut hard cataracts. The actuation mechanism can also be configured to lock the cutting element in position when not in use.

The tip includes a circular lumen with an outer diameter of 1.5 mm and an inner diameter of 1 mm. The tip includes 4 equidistant elliptical holes (2 top, 2 bottom) about 1.1 mm apart (mid-point to mid-point) 0.6 mm length and 0.4 mm width, 0.5 mm from the edge of the tip. The holes are extruded 90 degrees to the plane of the surface of the tip.

In some embodiments the cutting element takes the form of 4-0 nylon sutures with a circular diameter. The cutting mechanism, as configured for the present invention, is able to cut through all grades of cataract. The cutting element has a high flexural modulus to conform to the cross-sectional areas of different cataracts. Each end of the cutting element is threaded through a single hole on the tip. Rotating the tip rotates the loops. Both loops of the cutting element are symmetrical and deployed together. Both loops also have the same cross-sectional area (elliptical with 11 mm length and 4 mm height). In some embodiments, the loop closer to the cataract has a larger cross-sectional area. The cutting element as a whole, responds to actuation by the surgeons as one whole element—deploying and retracing as a unit together with the bridge. The cutting element surrounds/engulfs the cataract as it is deployed from the side. The cutting element captures the cataract through the vacant space between the loops and fragments the cataract. Internal facing edges of the loops of the cutting element stabilize the cataract as they move alongside it, preventing side movement during deployment. The cutting element naturally deploys in a direction straight from the tip. The cutting element can also have a slight angulation produced by the angled holes on the tip from where it is deployed. The loops of the cutting element do not contain any tension when deployed without obstruction

The bridge takes the form of a monofilament nylon 6-0 sutures with a diameter of 0.09 mm. The bridge can have a variable length from 3 mm to 5 mm. The location of the bridge can also vary based on desired treatment outcome. The bridge can be placed at variable locations of the bridge—i.e. it can be places symmetrically along the loop. For example, the bridge can be placed symmetrically on both loops (not participate in cutting the cataract) or diagonally (participate in fragmenting the central portion of the cataract) or symmetrically in the distal middle. Alternately, the bridge can be adhered to the cutting element (immobile). The bridge enables dynamic deployment of the cutting element and enables nucleus capture. Additionally, the bridge keeps the bottom edges of the loops together in one embodiment and the alternate edges of the loops together in another. In an embodiment with a diagonal bridge, the bridge cuts the central fragment into half based on its retraction trajectory. The bridge can be repositioned up and down the loops relative to the distance between the top and bottom as per the movement and position of the sliders. This changes where the restriction of the loops is located. The bridge can also be deployed up to 12 mm in length to allow capture of even the largest nuclei.

The bridge-loop junction can be made of a single knot. Any knot or other means of attachment known to or conceivable to one of skill in the art can be used. The bridge-loop junction is configured to be able to withstand cutting forces applied by the loops and bridge, when the loops and bridge cut through even the most mature cataract. The bridge-loop junction is generally immobile. In some embodiments, the bridge-loop junction is located symmetrically across from the other loop while in other embodiments it is located diagonally across.

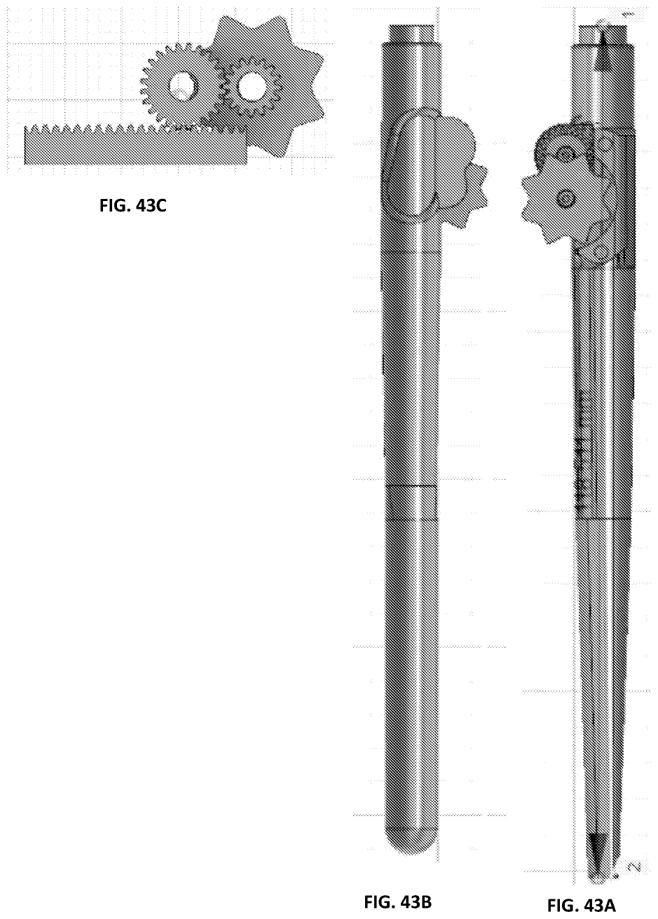

FIGS. 43A-43C illustrate views of a handle, gear body, and slider, according to an embodiment of the present invention. As illustrated in FIGS. 43A-43C the actuation mechanism can take the form of a rack and pinion. Alternately, the actuation mechanism can take any other suitable form known to or conceivable to one of skill in the art.

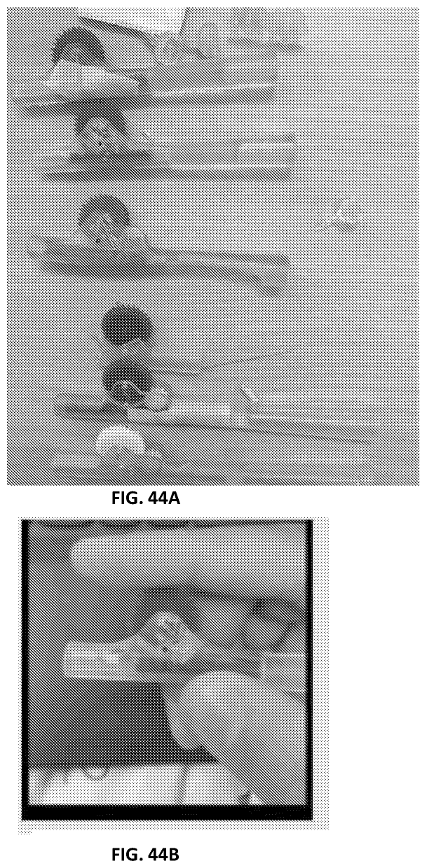

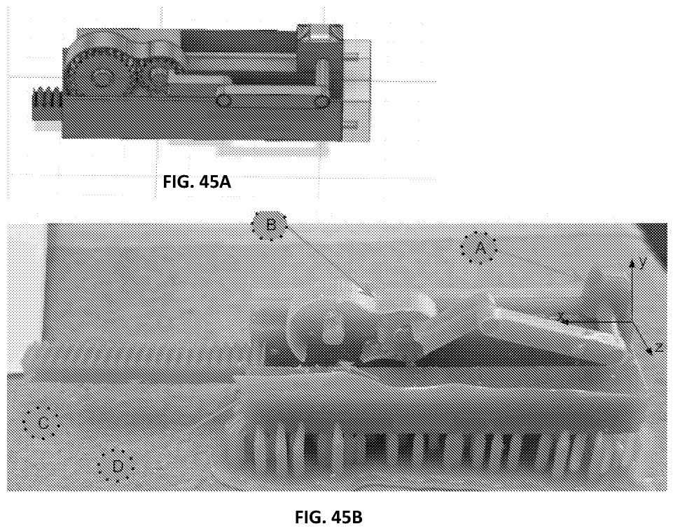

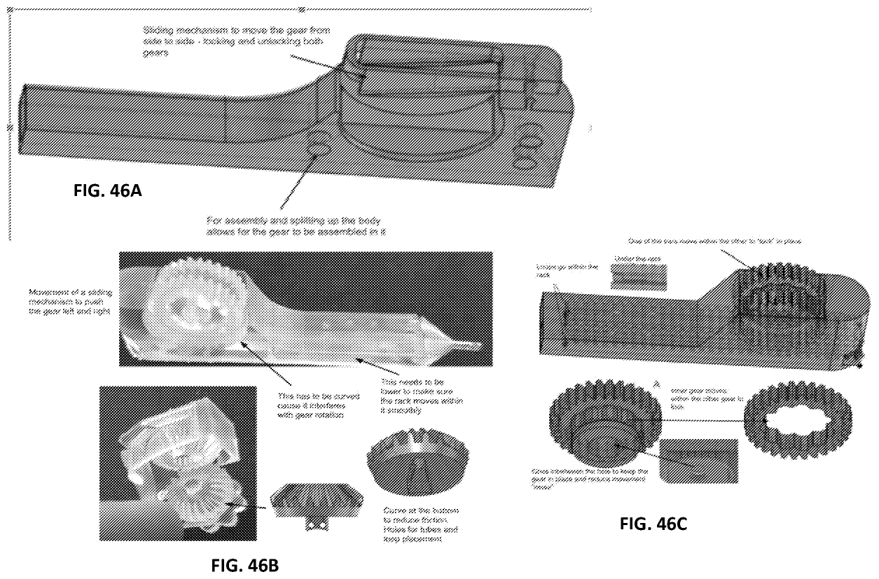

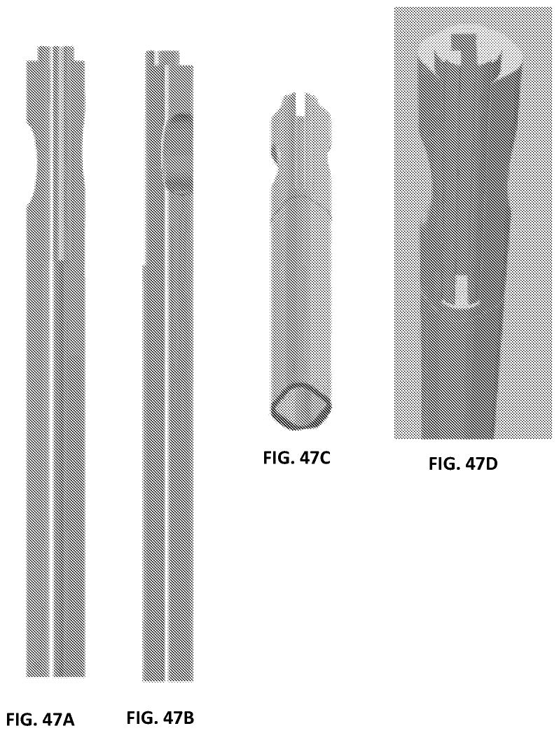

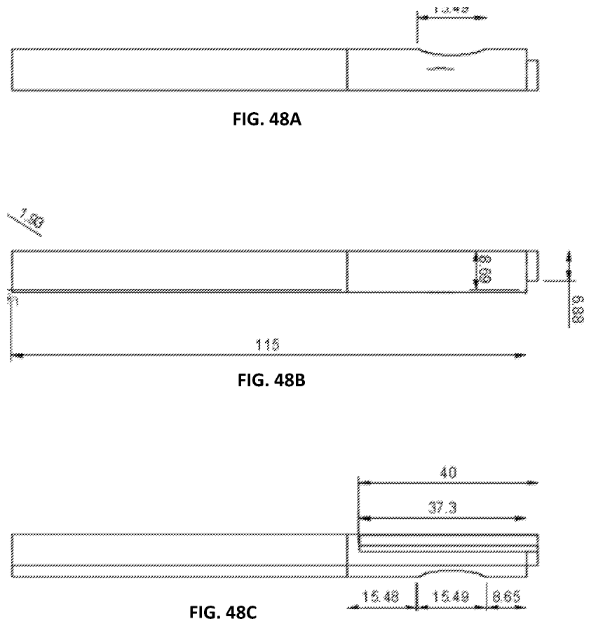

FIGS. 44A and 44B illustrate views of gear bodies, according to an embodiment of the present invention. FIGS. 45A and 45B illustrate perspective views of a slider crank mechanism, according to an embodiment of the present invention. Slider-crank mechanism with mechanical advantage and rack & pinion (Sliding motion, with mechanical advantage) & reduction to practice. FIGS. 46A-46C illustrate views of sliding mechanisms, according to an embodiment of the present invention. FIGS. 47A-47D and 48A-48C illustrate perspective views of slider body, according to an embodiment of the present invention. These figures illustrate variations on an embodiment having an actuation mechanism that takes the form of gear bodies.

Generally, as shown in the figures the gear body is between 80 mm and 120 mm in length. The gear body can include two circular gears and one rack. The gear assembly can include a driver gear and a follower gear. The follower gear has a greater number of teeth than the driver gear. The driver gear is attached to a tracking wheel that serves as the actuating element.

The body of the device can include divots to provide a location for the surgeon's finger(s). The divots can be positioned such that one is for the middle finger and the other for the thumb, formed based on positions of fingers in tripod grasp. In some embodiments the body is linear. In other embodiments the body is curved at the end from 5 cm away from the edge+tapering at the end of the tip 5 cm away from the end. The body can have a circular cross sectional shape or the cross-sectional shape can have a number of sides, such as 6, thereby forming a hexagonal cross-section.

In other embodiments, the actuation mechanism can take the form of a slider body. The slider body is disposed in a hollow area defined by the handle. The slider can be directly angled up. Divots located on the left of the device and at the bottom right are for the thumb and middle finger respectively. The slider would generally be actuated by the index finger. The length of the slider body is generally 5 cm to 12 cm, with a preferred length at 11.50 cm.

The cross section can have any number of edges. For example, with 0 edges the cross-section would be circular and with 6 edges it would be hexagonal. The shape of the handle can be linear with some curvature at the edge to enable the device to rest in the web between the index finger and the thumb. In some embodiments, there is a flat surface at the bottom to provide stability to hold the device and prevent undue rotation of the body. The flat surface can also provide a scaffold for the middle finger to rest upon, and an intuitive reference plane for the user as the bottom surface is on the same plane as the bridge that will be deployed within the anterior chamber. In some embodiments, weight is equally distributed across the device while in other bodies it was weighted forward and to the tip. Users preferred the weight being distributed to the front of the device.

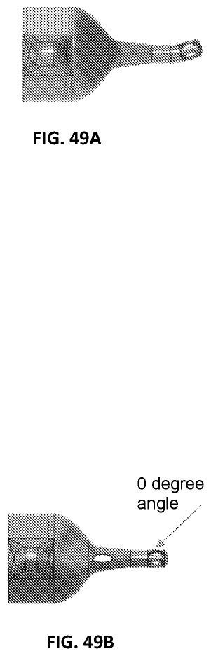

FIGS. 49A and 49B illustrate perspective views of another embodiment of a tip of a device, according to the present invention. The tip can have an angle from 0 degrees to 90 degrees that allows the edge of the tip to be inserted into the anterior chamber on the same plane as the iris. The angle can be placed right after the elliptical holes—at least 2 mm from the edge of the tip in the current embodiment. The vertical height of the tip (from the lowest point on the edge of the tip to the highest point

For a procedure using the device of the present invention, with a tip design shown in FIG. 49A or 49B the scleral-corneal incision is at least 2.75 mm. The incision can be made at the superior position or at the temporal position. If the incision is at 6 o'clock for the main incision at the superior position, there would be two paracenteses at the 3 o'clock and 9 o'clock positions. This enables surgeons to make a clean and accurate incision consistently that enables all the properties of a tri-planar incision. Through this, surgeons keep astigmatic outcomes predictable. Can be used with any intraocular lense (IOL) known to or conceivable to one of skill in the art. A procedure using the device of the present invention can use a foldable IOL—for <5 mm incisions; a PMMA IOL—for 4 mm or more incisions; or a Toric foldable IOL.

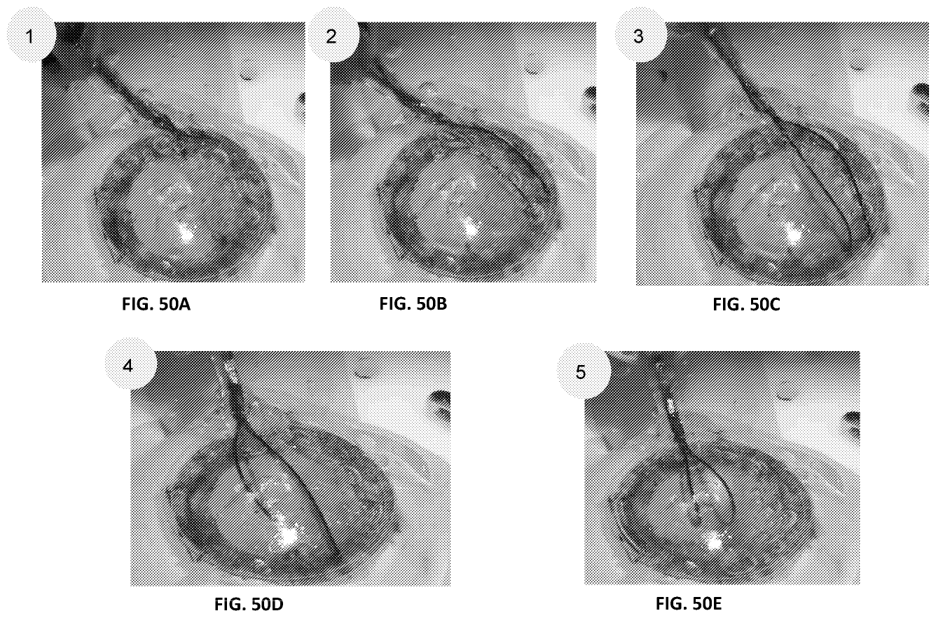

FIGS. 50A-50D illustrate an exemplary workflow, using a device according to an embodiment of the present invention. In a 3-piece fragmentation embodiment, where bridge is symmetrically positioned and below the cataract, a 3 mm scleral-corneal incision is used and 3 oblong fragments are created. In a 4-piece fragmentation embodiment, where the bridge participates in cutting the central fragment, 4 fragments are created, with a central piece cut in a 45-degree fashion, straight down the middle or in another angle near the equator. In a preferred embodiment of the device for generating 4 fragments, the bridge is in a diagonal orientation at 45 degrees. However, it is also possible to have other angles or a 0-degree angle at the mid plane of the cataract.

In a first step, the tip is inserted to the side of the nucleus and advanced as far as 2-3 mm into the chamber. The surgeon then ensures that the tip is on plane with the iris. The tip is rotated towards the surgeon to create a slight upward angle for the loops. The tip is inserted into the anterior chamber of the eye through a 3 mm incision and placed 1-2 mm away from the scleral corneal wound.

In the first step, the cutting element is comfortably docked within the tip allowing for smooth entry into the wound. The cutting element is positioned flush with the outer surface of the tip upon retraction allowing for smooth entry into wound. Further with respect to the cutting element, the nylon polymer does not deform when bent. The surface of the nylon is smooth (surface roughness characterization) and enables smooth deployment and retraction. Nylon conforms to the shape of the lumen it is forced within the tip. The nylon sutures that form the main loops have a circular shape. The nylon of the sutures forming the loops can have a diameter between 0.15 mm-0.05 mm diameter and a cross-section making the cutting element easy to deploy and store. The nylon forming the loops also conforms to the shape of the outer surface of the tip when retracted, to allow it to be flush with the outer surface of the tip. In the first step, the bridge is docked at the bottom edge of the tip (for straight bridges) or at a diagonal at the end of the tip (for diagonal bridges). The diameter of the bridge of 0.05 mm creates small cross-sectional profile. A smooth polymer material, such as nylon or PP, allows for easy docking. The bridge is flush with the length of curvature between the 2 holes at the bottom of the tip. In some embodiments, the diagonal bridge is flush with the diagonal edges on the surface of the tip. The attachment points of the bridge are docked at the periphery of the elliptical holes at the tip. Knots secure each end of the bridge to the cutting element, and determine a maximum allowed retraction of the cutting elements. The knot can take the form of any knot known to or conceivable to one of skill in the art, such as a non-slip knot. Alternately, a non-slip adhesive can be used. A diameter of knot is larger than diameter of elliptical holes on the tip.

In step one, the holes of the tip are placed 1 mm away from the edge of the tip, to prevent the loops from getting stuck within the scleral-corneal tunnel of the anterior chamber and becoming damaged and unable to deploy. A length of the tip is configured to limit the length that goes into the anterior chamber for added safety. The length of the tip also enables the surgeon to focus on deploying and manipulating the loops to capture the nucleus, and reduce chance of buckling. Tapering from the edge of the tip to plug the wound and prevent pressure loss while the device is used within the anterior chamber. The tip is configured to fit through a 2.75 mm corneal and sclera-corneal incision. The tip is inserted the anterior chamber of the eye through a 3 mm incision and placed 1-2 mm away from the scleral corneal wound. The tip is at the same plane as the incision created on the wound to reduce any trauma to the wound. The tip is first inserted straight into the wound and upon 1 mm-2 mm of being in the anterior chamber. The tip is angled to either the left or the right side depending on the comfort and preference of the surgeon. There could be up to 2 paracenteses at the 9 o'clock and 3o'clock position, with the main wound being 4 mm at a 6 o'clock position. If the surgeon angles the tip to the left, the instrument may also tilt towards the left. If the surgeon angles the tip to the right, the instrument may also tilt towards the right.

In step two, the loops are deployed slowly while maintaining contact with the nucleus. As the loops are deployed, the surgeon maintains visual contact of the top of the loop closest to the nucleus. As the loops get close to the other end of the cornea, the tip is swept towards the other end to bring the loops to the center of the nucleus. The cutting elements are deployed to the side of the nucleus to engage the side of the nucleus. The top of the loop glides along the top of the nucleus. The surgeon checks whether bottom portion of the cutting element is underneath the nucleus. Once the loop sufficiently engages the nucleus, the loops conform to the cross-sectional profile of the nucleus. A cross sectional area of the loop increases with the cross-section of the nucleus. The loops have an elliptical shape that generally matches the cross-section of the nucleus for ease of initial and subsequent capture.

The tip has an outer diameter of less than 1.5 mm, which enables it to fit at the side of the nucleus. The rigidity of nylon 4-0 allows loops to deploy in a straight manner. A perimeter of the loops can conform to the shape of the cross section of the nucleus it is capturing. Any heights from 2 mm (NS1) to 5 mm (NS5) of nucleus or any grade of cataract. The low flexural modulus of nylon allows the nylon loop to deform according to the geometry of the loops. The loops have a minimum cross sectional area of 0 mm², maximum follows an ellipse with a length of 11 mm and width of 5 mm=86.39 mm Upon deployment, the loops provide a space between the top and bottom that allows for engagement of the nucleus. Nylon has high coefficient of elasticity.

In step two, the bridge maintains the distance between the 2 loops, preventing entanglement. The bridge ensures a rigid scaffold between the 2 loops, enabling the loop closest to the edge of the nucleus to engage the side of the nucleus well by maintaining the structure and volume of the space enclosed by the loops. Both a straight and diagonal configuration of the bridge allow unrestricted deployment of the cutting elements in order to initially capture/contain a small portion of the nucleus. This facilitates subsequent encapsulation of the nucleus.

In step two, the tip maintains some distance between the 2 loop edges at the top and the 2 loop edges at the bottom. The tip creates a volume of space for the loops to glide and capture the nucleus. The tip prevents the edges of the loops from being entangled, and ensures that the configuration of the loops and the bridges are kept while they capture the nucleus. The tip provides additional volume (more than the cataract being captured) within the space enclosed by the loops to aid with adjusting the position of the loops on the cataract. The tip ensures both/all cutting elements are deployed symmetrically. The holes on the tip are equidistantly and symmetrically spaced apart on the surface of the tip. An angle of deployment of the loops is configured to the rigidity and flexibility of the 4-0 cutting elements. The top portion of the cutting elements exit from holes which are spaced anteriorly to the holes through which the bottom portion of the cutting elements exit. The holes through which the cutting elements exit are sufficient in size only to allow one wire to pass through the hole.

Further with respect to step two, as the cutting elements are deployed, and the surgeon confirms the nucleus is engaged with the proximal cutting element, the surgeon turns the tip and retracts the tip from the chamber (while keeping the tip within the chamber).

In step three, the surgeon sweeps the tip to the other side. This motion assists in bringing the loops over the nucleus and positioning them at the center of the nucleus. The surgeon further makes sure to gently have the loops glide along the top of the nucleus. In step three, the device begins to capture the nucleus by deploying the loops further and capturing more of the nucleus, moving the loops from the side to the midline of the nucleus.

In step three, the loops hug and glide along the top and bottom surfaces of the nucleus. The loops match the contour and cross sectional profile of the nucleus. A cross sectional area of the loop increases with the cross-section of the nucleus. Loops are rigid enough to respond to the actuation by the slider and gear body, there is no lag in response. Loops provide haptic feedback to the user. Loops create and maintain the vacant space between the top and bottom half to neatly encapsulate the nucleus with less friction. Loops can smoothly glide along the nucleus. Surgeons are able to note the location of the loops once the top half is over the nucleus. It should also be noted that the loops have a color that is visible in the microscope (black, bright colors, smooth/shiny surface).

In step three, the bridge keeps the top end of the loops together as the cutting element is being deployed. The bridge creates asymmetry between the bottom and top edges. This causes tension/torque in the material and causes an inward tilt of the bottom edge, towards the nucleus. The effect of the bottom edge of the bridge hugging the nucleus pulls the top edge downwards and causes it to stick close to the nucleus. The bridge provides trajectory and unidirectionality of deployment. The bridge glides along the top surface of the nucleus as it is deployed. The bridge also has a connection to the top half of the tip allows the bridge to engage the surface of the nucleus easily and glide along it.

In step three, the cutting element is deployed at an angle, allowing it to capture the nucleus with more ease. Constricted movement of free wires enables translation forward. This function is facilitated by the holes in the tip being angled 30-50 degrees to the central line through the tip. The holes are close to the tip edge, 0.5 mm away. The holes are 0.4-0.6 mm in diameter, allowing for smooth deployment and snug enough to the CE. A cylinder at the back reduces the likelihood of the loops buckling within the tip and outside, especially at the tip-body interface. An inner lumen of tip contains just enough space for all the wires. The nucleus is captured by sweeping the loops from the side to the midline of the nucleus

In step three, the loops are deployed to be larger than the area covered by the captured nucleus. The tip is gently rotated towards the centerline, and the loops are tightened to capture and center the nucleus. Gently pushing or pulling the loops along the top surface of the nucleus towards the midline of the nucleus with a sinskey or another blunt instrument. The tip is retracted as the loops are expanded to allow loops to capture the nucleus and occupy the chamber

In step four, the loops are positioned at the center of the nucleus, and the slider is retracted to grasp the nucleus and hold it in place. The loops are guided easily to the center of the nucleus. The loops respond to the motion of the tips (swivel and rotation). The loops can smoothly move across the nucleus without getting caught in it. The loops maintain their position according to the plane of the nucleus. The loops can manipulate and adjust the plane of the nucleus to keep it aligned with the plane and angle of the tip by retracting the actuation element to “grab” the nucleus. The nucleus can be repositioned around the AC to the best position for fragmentation. There is minimal lag between the leading and following cutting element(s).

In step four, the bridge keeps the top end of the loops together as the cutting element is being deployed. The bridge creates asymmetry between the bottom and top edges. This causes tension/torque in the material and causes an inward tilt of the bottom edge, towards the nucleus. The bottom edge of the bridge hugging the nucleus pulls the top edge downwards and causes it to stick close to the nucleus. The bridge provides trajectory and unidirectionality of deployment. The bridge glides along the top surface of the nucleus as it is deployed.

In step four, the cutting element is deployed at an angle, allowing it to capture the nucleus with more ease. Once satisfied with centering, the surgeon can hold the tip and handle in position, and retract with slider or wheel. Surgeon may utilize a sinskey in a 6'o clock position to gently keep the nucleus in place while the loops are retracted (if needed).

In step five, the surgeon slowly retracts the slider using an index finger or thumb (depending on the preference of your hand position) to cut the nucleus. Three fragments are created with the two loops and a lower bridge. Four fragments are created with two loops and a diagonal bridge—the fourth fragment is created by cutting the central fragment into two pieces. The loops are retracted to fragment the nucleus into three or four fragments. In some cases, the cataract could be fragmented into five pieces.

In step five, the cutting element maintains a straight trajectory through the nucleus capture and fragmentation process. The cutting element does not slide at the initial point of cutting the nucleus. The cutting element can be visually seen. The cutting element withstands the tension required to cut a mature (>NS4 cataract). Therefore, the cutting element is able to cut through all grades and types of cataract. A 4-0 loop size, more specifically 183 microns of loop diameter is the most suitable for engaging the nucleus from the side and capturing the nucleus. Loops with a smaller diameter than 4-0 tend to be more flaccid and flexible within the anterior chamber. The additional flexibility (with reduced rigidity) makes reduces the ability of the loops to glide along the cataract in a controlled and predictable manner. This is unsuitable for cataract surgeons. Loops with a larger diameter than 4-0 tend to be more rigid and bulky. Additional bulk takes up too much space in the AC when deployed. Added rigidity pushes the nucleus away rather than conform to the surface geometry and side profile of the nucleus to encapsulate the nucleus. 6-0 nylon (specifically 90 micron) for the bridge is most suitable to allow the space between the two loops to be maintained, and to allow the following loop to move with the leading loop smoothly during capture, A 3 mm bridge length results in pieces that are approximately 3 mm each for a 9 mm nucleus. A 5 mm bridge length oriented at 45 degrees results in 4 fragments, with two central fragments between 2.5-3 mm in width and side fragments 3 mm or less in width for a 9 mm nucleus.

The cutting element is formed using an etched grid with appropriate markings and grooves to assemble cutting elements with the bridge at the appropriate distance, position, and angle. A continuous suture holder can be threaded to achieve large-volume production of cutting elements and bridge. Cutting of a nylon sheet with thickness equivalent to the 4-0 and 6-0 bridge diameters (183, 90 microns respectively) via laser cutting or other appropriate methods. Joints can be ultrasonically welded.

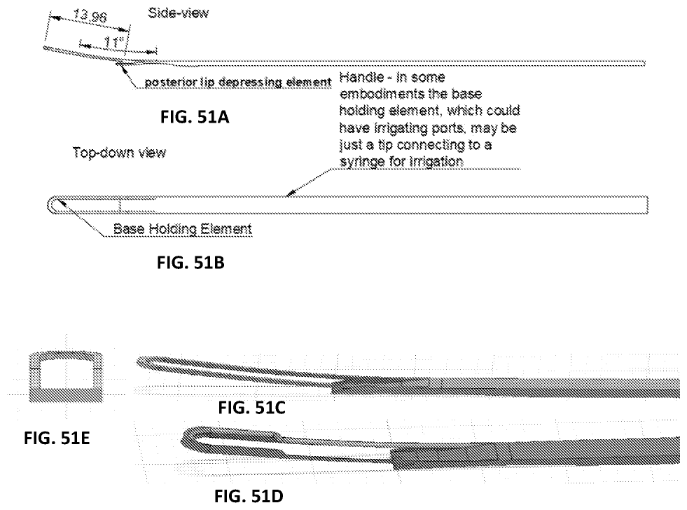

FIGS. 51A-51D illustrate views of fragment removal instruments, according to an embodiment of the present invention. For fragment removal, the mid portion of the fragments are removed first. In some embodiments, the mid portion will be cut diagonally across its length into two halves. These halves will then be removed through a variety of ways some of which includes, a suction element to pull the pieces out, a Vectis of at least 2 mm width and above, an irrigating Vectis of at least 2 mm width, injecting viscoelastic within the chamber to express the fragments out through the incision (sclera corneal or corneal). The side fragments are then removed following the removal of the fragments in the central piece. In this case similar techniques are used as in the removal of the central pieces. Alternative removal embodiments listed below can be used to remove the fragments from the AC. In some instances, the nucleus is engaged by inserting base below the nucleus. The novel element is a posterior lip depressing element, which remains outside and away from wound, until the nucleus is engaged, at which point, it is gently pushed forward to press on the posterior lip, opening the wound and facilitating removal. In some embodiments, the base holding element can have irrigation ports or can just be a tip connected to a syringe for irrigation.

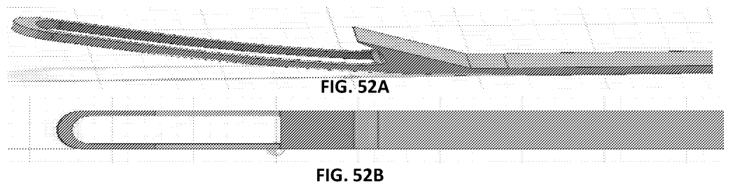

FIGS. 52A and 52B illustrate views of an alternate embodiment of a fragment removal instrument, according to an embodiment of the present invention. The base is inserted into the wound, and during maneuvering, the top wedge is positioned over the anterior lip of the wound, preventing over-entry into the wound. At the time of removal of the fragments, the wedge is pulled back and the posterior lip of the wound depressed to facilitate a gradient within the wound of the fragments. The angle or curvature of the base may be concave to create more space to hold the nucleus.

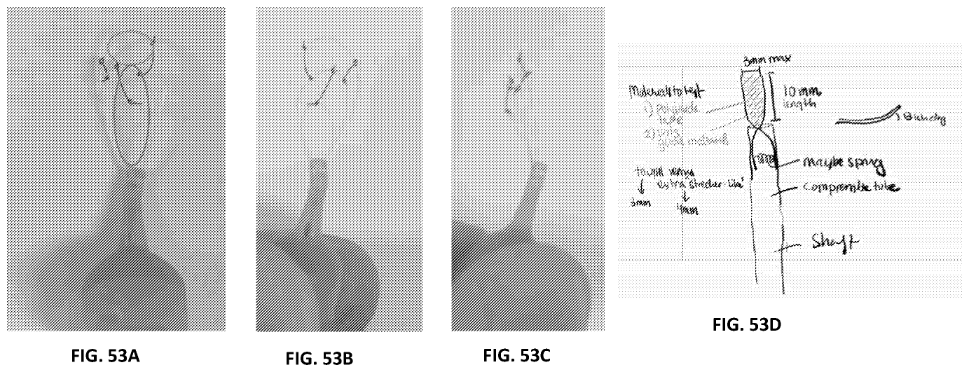

FIGS. 53A-53D illustrate views of removal instruments and tip attachments, according to an embodiment of the present invention. A flexible sheet of plastic at the edge of the Simcoe will conform to the shape of the fragment being captured to improve the suction force on the fragment. A thin, soft and flexible sheet operated by a cross-tweezer mechanism and fitting through a small incision—operating as a “stretcher” can also be used to capture and remove the cataract. A grasping net (two concentric loops oriented parallel to plane of iris, connected by a vertical bridge(s) to one larger loop—loops would be inserted to their side, rotated below the nucleus and tilted upwards. Fragment is engaged within the opening between the loops, loops are tightened and fragments are retracted. Grasping loops—a variety of grasping loops made of soft polymer such as nylon may be used to hold and remove the lens fragments.

In some embodiments, a Simcoe cannula is used to irrigate and aspirate the fragments out. The Simcoe cannula can have a hold on the fragments with the suction and pull the pieces though the wound. Using a tip that has a flexible sheet at the tip, like a parachute, the nucleus fragment is placed within the sheet that encapsulates the fragment. The sheet is pulled back to get flush with the fragment and take the shape of its geometry. Once pulled back, suction is applied to seal the sheet to the fragment preventing it from moving around. The fragment is then pulled through the wound. Sheet allows minimal disturbance of other ocular tissue due to its flexible, wavy and thin nature.

Once the bridge is retracted halfway or ¾ of the cataract, the device along with the loops and the nucleus and its fragment will be pulled out of the AC. This will result in the mid fragment being pulled out with the loops and the bridge. A port at the wound (scleral corneal or corneal incision) maintains the maximum opening of the wound for various incision sizes from 2.75 mm to 4.5 mm. Therefore, the cross-sections are from 2.4 mm² and 6.44 mm² respectively. The port conforms to the outer perimeters of different incision sizes from 2.75 mm to 4.5 mm. Maintains the pressure within the AC to about 40 mm Hg through passive injection of viscoelastic or saline—enclosed system of 40 mmHg. Silicone or other soft material shaped as a pucker with a fixed size hole or cross-slits that sits in the wound—different pucker sizes for different wounds—each one having a fixed size opening for fitting the tip snugly without any viscoelastic leak and eliminating need for expanding incision after entry

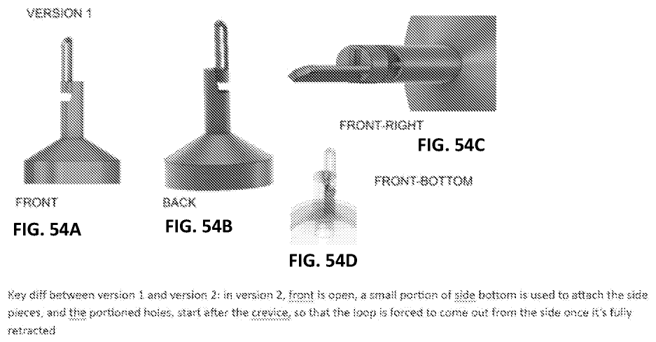

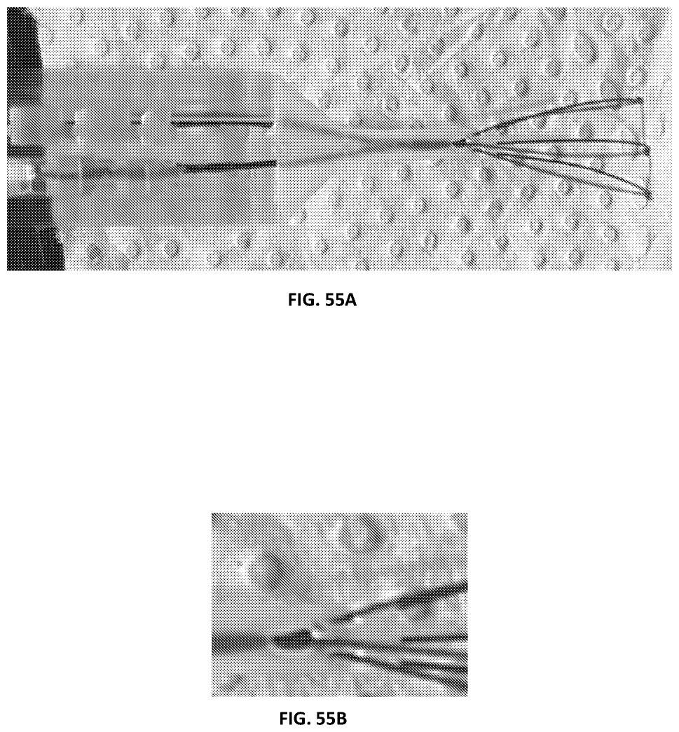

FIGS. 54A-54D illustrate views of a tip and hub of a device, according to another embodiment of the present invention. FIGS. 55A and 55B illustrate views of a tip and hub of a device, according to the embodiment of FIGS. 54A-54D. The device includes a channel to re-set the loop to the edge of the tip following retraction. A key difference between version 1 and version 2 is that in version 2 the front is open to a small portion of the side bottom is used to attach the side pieces and the portioned holes start after the crevice so that the loop is forced to come out from the side once it is fully retracted. In the embodiment of FIGS. 54A-54D and 55A and 55B. Each loop is actuated by its own slider. There are three loops and three exit holes, and each loop is actuated independently upon deployment. All of the slider/actuators are retracted at the same time to fragment the cataract.

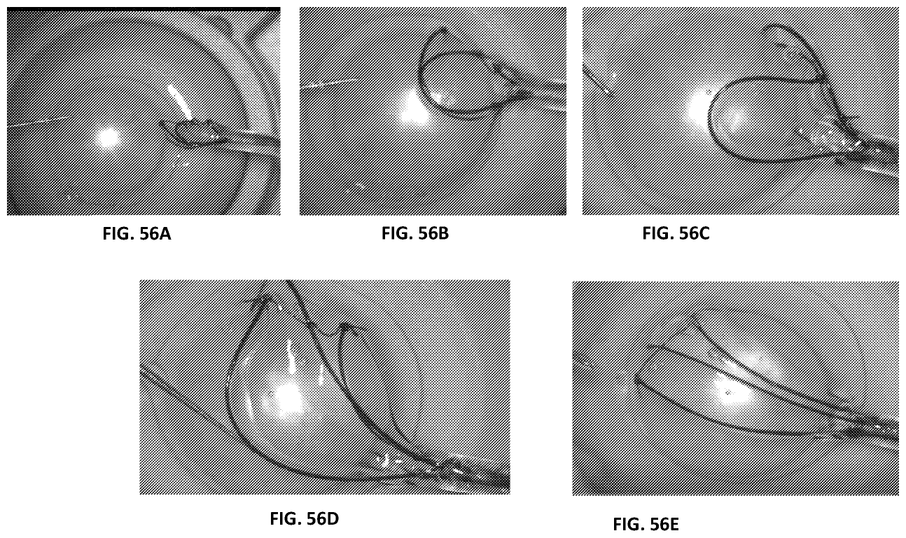

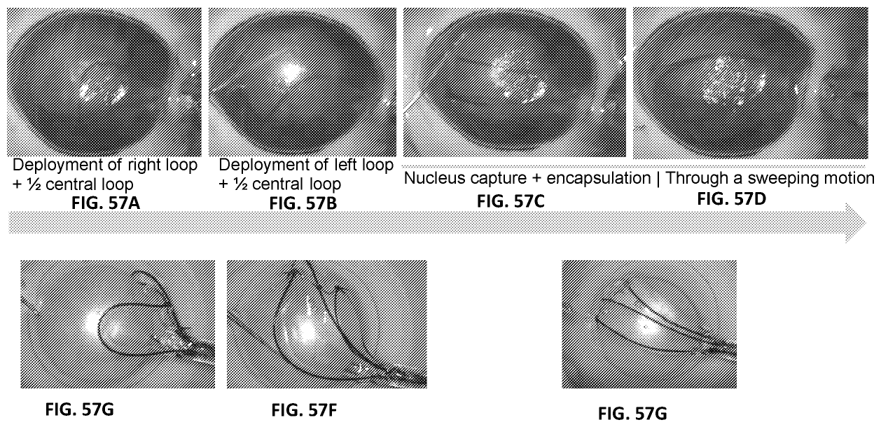

FIGS. 56A-56E illustrate a workflow for fragmentation of a cataract, using a device according to an embodiment of FIGS. 54A-54D. FIGS. 57A-57G further illustrate a workflow for fragmentation of a cataract, according to an embodiment of the present invention. The tip is inserted within the AC. A slider connected to one loop is deployed. The loop curves in the direction of the bridge attachment. The curvature of the loop creates a vacant space between the loops to then capture the nucleus. The loop continues to be deployed to glide over the top and bottom of the cataract to engage it and create a scaffold to hold the cataract in place. The scaffold is built/deployed to the side of the cataract—the side away from the deployed loop. The loop continues to be deployed to glide over the top and bottom of the cataract to engage it create a larger scaffold. The other two loops to the right side remain docked within the tip. The loop acts like a glove to support the bottom and top of the cataract preventing it from moving too close to the endothelial cells of the cornea. The loop also prevents the cataract from moving down towards the posterior capsule. The two other loops connected to the same actuator are deployed. This method results in the encapsulation of the nucleus from the right to the left side. Loops glide along the periphery of the nucleus and situate themselves equidistance along the width of the cataract as defined by the length of the bridges between each loop.

A secondary instrument can be used to nudge the nucleus at its periphery and keep it in place during encapsulation. At all times the device tip is at the wound. The two loops continue to be deployed allowing the entire system of the three loops to encircle the nucleus and capture it. Deployment of the loops enable the device to be symmetrically positioned on the cataract. Once the loops are positioned well, the cataract can be cut into 4-6 fragments by retracting the loops.

In this embodiment, the tip has 3 holes that are placed side by side on the bottom and top of the tip. The three holes can have varying configurations. In one configuration the holes are side by side. Alternately, the holes can be positioned in a triangular configuration with one in front of the rest or a reversed triangle with one below the other two. The hole width is from 0.1 mm to 1 mm and length 0.1 mm to 1 mm. The hole shape is generally circular or elliptical. Distance from edge of tip is 0.1 mm to 3 mm—OD and ID, angle of the tip are the same as mentioned in other embodiments.

In this embodiment, the cutting element is formed from 4-0 nylon sutures with a circular diameter of 0.15-0.199 mm. The cutting element is able to cut through all grades of cataract. The cutting element has a high flexural modulus to conform to the cross-sectional areas of different cataracts. Each end of the cutting element is threaded through a single hole on the tip. Rotating the tip would rotate the loops. Both loops are symmetrical and deployed together. Both loops have the same cross-sectional area (elliptical with 11 mm length and 4 mm height) In some embodiments, the loop closer to the cataract has a larger cross-sectional area. The cutting element, responds to actuation by the surgeons as one whole element—deploying and retracing as a unit together with the bridge.

In this embodiment, there are four bridge loop junctions. It should be noted that there could be more junctions depending on the number of bridges. The bridge is 0.1 mm to 4 mm apart. The device of this embodiment could be made with 1 bridge between each loop or any number of bridges at the edge of the loops. Bridge can be symmetrically placed across the loop or diagonally—orientation from 180 to 90 degrees (clockwise or counter). As mentioned in other embodiments: The bridge is composed of monofilament nylon 6-0 sutures. The bridge has a variable length from 3 mm to 5 mm. The location of the bridge can be variable—can be placed symmetrically along the loop. The bridge can be placed symmetrically on both loops (not participate in cutting the cataract) or diagonally (participate in fragmenting the central portion of the cataract). The bridge is adhered or knotted to the cutting element (immobile). Knots are spherical and from a diameter of 0 mm (does not protrude out) to 2 mm. The knots are made, in some embodiments, by spreading a bonding agent such as in some cases Loctite on the surface and dropping a small volume of cyanoacrylate (CA) on the knot to fill in any gaps between the knot created by the 6-0 suture on the 4-0 suture in that order. Any medical grade CA can be used.

For the cutting element, one of the loops is deployed and curves towards the other 2 loops that are docked within the tip. One of the loops creating the curvature starts creating a space for the periphery of the cataract to fit within the loop. The loops provide a good resolution of actuation. Cross-sectional area increases as the loop is deployed. A top edge of the loop glides over the top surface of the cataract while the bottom glides under, while having the middle loop 3 mm away from the tip via the bridge. Nylon does not exert undue tension and translates the force to more flexible parts on the loop—those locations that are unrestricted.

The bridge keeps the loops linked together. The bridge maintains the tension between all 3 loops. The bridge enables the loops to function independently and be controlled independently with each actuator—1 actuator for the left most loop and another actuator for the middle and right loop. The bridge divides the left most loop into top and bottom edges that glide along the top and bottom respectively.

Two loops on the right are deployed and capture the nucleus from the right to the left. Following capture and symmetrical positioning of the loops, the nucleus will be fragmented into six pieces. Two of the loops are deployed to capture the nucleus and straighten out all three loops. The whole cutting element moves from the right to the left to capture the nucleus. The loops glide along the nucleus and create a cross-sectional area to capture the nucleus. The loops maintain their position while docked in the tip. The bridge keeps all of the loops equidistant from each other at all times. The bridge enables the translation of the two loops to the distal end of the nucleus by following the curvature of the first loop deployed.

Other embodiments that have a mechanism to maintain loop position as they are retracted include 1) reinforcement of the loop with a stiffer secondary polymer or metal wire that is braided around some or all of the length of the loop, 2) a mobile bridge-like element that moves along the length of the loops, and 3) an elastic bridge-like element. In another embodiment, all cutting elements can have a trajectory that is perpendicular to the corresponding surface that the cutting elements first come in contact with, through the use of internal tracks in the body with transiently interlocking sliding mechanisms that allow independent trajectories of each cutting element while maintaining synchronicity of movement.

In each of these embodiments, the body can contain a hollow lumen that is attached to a syringe which can be pulled to remove fragments or provide suction. In other embodiments, the lumen and syringe can contain viscoelastic or other irrigating solution which is released into the AC at various times in the procedure during insertion, deployment, retraction or removal of the fragments, per the surgeon's preference.

For any of the embodiments, the cutting elements can be re-arranged around the flaps or prongs or guide rails of the tip manually by the surgeon or through the use of a secondary tool, such as one where two edges of a tweezer like instrument are engaged inside the cutting elements while they are retracted, such that the cutting elements re-dock around the flaps. The present invention includes several embodiments capable of manual cataract encapsulation, fragmentation, and optionally, removal, which are depicted below.

In each case, loops can be made of metals, such as stainless steel, copper, nitinol, or of other medical grade, biocompatible, sterilizable materials, including nylon, polyvinylidene fluoride, silk, polypropylene, and optionally coated. With respect to any of the cutting loops of the present invention, it is possible to use a number of different materials with varying diameters and cross sectional shapes. In some instances, it is possible to use super-elastic nitinol wire with varying cross-sectional diameter and geometry. In some instances, the cross-sectional diameter can further vary along the length of the wire. The diameter of the wire can be between approximately 0.003 inches to approximately 0.005 inches in diameter. The cross-sectional shape of the super-elastic nitinol wire can be round, flat or any other geometry known to or conceivable to one of skill in the art. The loops can alternately be formed from any other material known to or conceivable to one of skill in the art, including but not limited to other shape memory metals, steel, metal alloys, nylon, polymers, or other plastics or elastomers. If nylon is used, it can take the form of a nylon filament in a range between 6-0 to 2-0 suture sizes, which is approximately 0.07 mm to approximately 0.3 mm. Other non-metal, cross-sectional loop diameter ranges from approximately 0.3 to approximately 0.04 mm.

In other embodiments, the wires are cured on a flat plane with circular diameter of 2-4 cm or cured on a circular plane with a maximum diameter of 12 mm. Loops can be braided with a combination of materials. The device can be inserted through a corneal, scleral, sclero-corneal, or limbal incision, and may be accompanied by assistive/complimentary devices that go through the main incision site or through another incision as deemed necessary by the user.

Novel elements include the loop design, loop composition, loop dimensions, loop deployment mechanism, tip design, tip composition, actuation mechanism, hollow shaft and tip structure, openings in the tip and passive elements that stabilize and direct the movement of the cutting elements during deployment, cataract encapsulation, and fragmentation. These structural elements enable creation of unique fragment types and novel methods of use of the device, including location of use, methods of fragment creation, including via repeated deployment and retraction of cutting elements, and methods of cataract fragment removal, including through the lumen of the hollow tip and shaft.

In preferred embodiments, the cutting elements, tip and passive elements act synergistically, resulting in the cutting elements' lateral motion along the sides of the cataract toward the centerline as they simultaneously expand towards the distal ends of the cataract. In other embodiments, at least one cutting element is positioned along each side of the cataract, laterally or horizontally, and moves synchronously with the other cutting element(s) medially towards the centerline of the cataract. This approach generates a symmetric force to support the cataract as it is captured by the cutting elements and prevents nucleus tilt in the lateral and anterior to posterior planes. In many embodiments, the cataract may be supported independently by at least two cutting elements or in combination with a base. Both elements in these embodiments are positioned symmetrically from the centerline of the embodiment in order to create equal and opposite moments to prevent the tilt of the nucleus and bear an equal weight of the cataract.

In many embodiments, the anterior-posterior and lateral plane of the cataract is maintained during the capture and fragmentation of the cataract due to the equal distribution of the force in the cutting elements and the supportive normal force from the cutting elements and the base. In some embodiments, the cutting elements are connected to each other via at least one bridge between two cutting elements that keep them separated by a pre-defined maximum distance at certain points on the cutting element. This maintains the distance between cutting elements and allows for the creation of cataract fragments with a width equal to or smaller than that of the bridge.

In some embodiments, there is a tip consisting of at least one central lumen, from the front to its back, and at least one exit and entry point, a hole of varying shape and geometry on its surface per cutting element, with each cutting element being individually connected to the central lumen(s) through the hole(s) in the tip. In some embodiments, the channel between the exit/entry points and the central lumen of the tip is angled (0-45 degrees) to the surface of the tip while the entry and exit points of any one cutting element are at least 0.05 mm apart from each other (in the anterior-posterior direction).

In many embodiments, the entry points of the cutting elements are positioned in the same anterior-posterior plane, but spaced apart in the lateral plane from each other, thus spacing apart the individual cutting elements. In some embodiments, the entry point is not positioned in the same lateral plane as the exit point, creating an angle on the cutting element that is dependent on the relative position of the entry and exit point. In some embodiments, passive elements initially assist in deploying the cutting elements laterally to the centerline of the cataract followed by medial movement towards the centerline, all while maintaining synchronicity between cutting elements throughout the operation.

In many embodiments, the device can be directly inserted and used without requiring surgeon manipulation or rotation of the body of the device. In some embodiments, the cutting elements may be used to capture, re-orient and re-fragment the cataract in a variety of orientations. In many embodiments, there can be a lumen that is available for suctioning or retrieving cataract fragments. In some embodiments, the tapered surface between the distal and proximal ends of the tip creates a plug to prevent liquid material or any other matter from exiting the wound, maintaining the anterior chamber, and preventing complications. This prevents a drop in intraocular pressure within the eye while the device is used.

In many embodiments, the cataract can be fragmented into at least three pieces with each piece having a width of <3.5 mm. Size of the fragments, will vary based on the overall size of the cataract and the number of fragments. However, it is to be understood that the number of fragments is dependent on the embodiment of the device and the treatment circumstances, including the geometry, shape, and movement of the cutting elements, and all fragmentation patterns are considered included with the present invention. Generally, it is desirable to fragment the cataract into at least two fragments. Preferably, the cataract is broken into three to four fragments. In some embodiments and under certain treatment circumstances, it may be desirable or necessary to create more than three to four fragments, as would be known to or conceivable to one of skill in the art.

The fragmentation device embodiments described may be used to encapsulate and fragment cataracts following insertion of the tip through an incision ranging from 2.5-6.5 mm in the eye into the anterior chamber which contains a prolapsed cataract. In many embodiments, the tip is angled to the left or right of the cataract, along its equator, wherein the actuating mechanism is used to deploy the loops to the side of the cataract, wherein the tip is simultaneously angled towards the centerline while it is brought back closer to the wound as the cutting elements capture the cataract in the anterior chamber, wherein the actuating mechanism is retracted to retract the cutting elements causing the cataract to fragment into 3 or more pieces. Following removal of the cataract fragments, an optic of a suitable size comprising foldable IOLs, foldable Toric IOLs, and a rigid PMMA IOLs can be deployed through the same incision. The cutting element may include of one or multiple loops of the same or different material than the other loop or loops. An adjunct instrument may be inserted through the side port or paracentesis, located at any meridian along the corneal limbus, and used to push the cataract into the loops or position the components of the cutting element symmetrically along the encapsulated cataract. In some cases, the device tip itself may is inserted into the paracentesis and cutting element deployed to capture any grade and type of cataract as it is prolapsed into the anterior chamber of the eye. The cutting element may conform to the cross-sectional geometry of any grade and type of cataract at any point on the cataract. The cutting element may also glide along the surface of the nucleus in order to position the loops symmetrically on the cataract to prepare it for fragmentation. In some embodiments, the components of the cutting element are deployed to a cross-sectional profile greater than the cross-sectional profile of the cataract and the tip of the device is rotated clock-wise or anti-clockwise to position and center the components of the cutting elements along the surface of the cataract. The cutting element may be retracted partially and then the tip is pulled out to simultaneously fragment and remove the cataract fragment or fragments through the incision. Alternatively, the components of the cutting element are retracted to a cross-sectional profile equivalent to the cross-sectional profile of the cataract in order to grasp and position the cataract by rotation or translation of the tip within the anterior chamber of the eye without cutting the cataract. The cutting element may be deployed and used to capture cataract fragments, and retracted to further fragment the cataract. Fragments from the first fragmentation may also be further fragmented to create smaller fragments to be removed through at least a 3 mm incision. The cutting element may also be utilized to facilitate removal of the fragments. Notably the tip of the device can be rotated and the cutting elements disengaged from the cataract at any point during the procedure.

Notably, the cataract fragmentation embodiments described may be used in a variety of ocular surgeries, particularly those in which a tissue or implant may require fragmentation or removal. For example, the device may be used to cut implanted optics, such as an intraocular lens.

The embodiments described may be used in several different ways in order to enable successful cataract surgery. The cutting element may be placed to the side or below cataract fragments and the cataract pushed into the loops, and the cutting elements pulled out without retraction through the incision to remove the fragment(s). A suture attached to the superior rectus may also be used to provide countertraction to assist in the removal of fragments from at least a 3 mm incision. During the procedure itself, dispersive, cohesive or a combination of dispersive and cohesive viscoelastic may be used within the anterior chamber of the eye to maintain intraocular pressure of the anterior chamber and coat the corneal endothelium to deploy, capture and fragment the cataract in a safe and efficient manner. In some cases, an anterior chamber maintainer may be used during the procedure to maintain intraocular pressure between 20-60 mm Hg and direct the fragments towards the incision by utilizing fluid flow into the chamber upon depression of the lower lip of the wound.

The cutting elements described can be used on all sizes, grades, and hardness of cataract in practice. In many embodiments, upon fragmentation, the cataract fragments are aligned to the wound for facile removal.

These features and exemplary implementations are not meant to be considered limiting, and any features, materials, or variations on these embodiments known to or conceivable to one of skill in the art could be used. Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

1. A device for cataract surgery comprising:
a handle;
a cutting element comprising at least two loops, wherein the cutting element is deployed to capture and fragment a cataract in an anterior chamber of an eye;
an actuating mechanism disposed within the handle, wherein the actuating mechanism includes an actuating element disposed on a surface of the handle and wherein the actuating mechanism is configured for deploying or retracting the cutting element; and
a passive element configured to facilitate movement of the cutting element during deployment or retraction of the cutting element.

2. The device of claim 1 further comprising a tip coupled to the handle, wherein the tip defines holes through which the at least two loops of the cutting element exit.

3. The device of claim 2 wherein the holes through which the at least two loops of the cutting element exit are positioned in a variety of configurations along the surface of the tip.

4. The device of claim 2 wherein the tip defines a center lumen through which the cutting elements translate.

5. The device of claim 2 wherein the tip is sized to be inserted through at least a 3 mm incision in the eye along any position at or between the superior and inferior positions of a sclera, limbus and cornea.

6. The device of claim 2 wherein the tip and handle have a hollow central lumen to facilitate removal and/or collection of fragments.

7. The device of claim 2 wherein the cutting element, passive element and tip act synergistically, resulting in the cutting element's lateral motion along the sides of the cataract toward a centerline of the cataract as the cutting element simultaneously expands toward a distal ends of the cataract.

8. The device of claim 2 wherein the tip further comprises guides or guiding geometry that position the cutting elements around the cataract.

9. The device of claim 1 wherein the cutting element comprises at least one connector of varying lengths in which each connector is attached at a single point on at least two loops.

10. The device of claim 9 wherein the connector participates in cutting the central fragment dependent on the height of the cataract and angle of the connector in relation to the loops of the cutting element.

11. The device of claim 10, wherein the cutting element further comprises two or more connectors, wherein the cutting element is sized to hold a cataract fragment for removal.

12. The device of claim 10, wherein the connector, when retracted, remains partially exposed from the edge of the tip.

13. The device of claim 1 wherein the cutting element is formed from one or more loops of the same or different biocompatible metals or polymers, wherein the material is configured to conform to the cross-sectional geometry of any grade or type of cataract or intraocular lens (IOL) and wherein the material is configured to withstand the forces required to fragment any grade or type of cataract or IOL.

14. The device of claim 1 wherein the cutting element is configured to be disengaged from the cataract or IOL at any time.

15. The device of claim 1 wherein the actuating element comprises a slider.

16. The device of claim 1 wherein the actuating element comprises a wheel or rotational element that translates the cutting elements forward and backward further comprising of a gear mechanism with two or more gears of variable ratios.

17. The device of claim 15 wherein the actuating element is connected to a mechanical advantage system to generate more force than a simple linear actuator during retraction of the cutting element.

18. The device of claim 1 further comprising an active or passive supporting element that facilitates stabilization of the cataract and fragment removal.

19. A device of claim 1 further comprising a passive or active element within or on the handle that when activated, dispenses saline, viscoelastic material or other aqueous substance within the anterior chamber of the eye through channels within the handle and/or tip.

20. A device of claim 1 wherein the cutting elements and passive elements encapsulate and direct the fragments of the cataract to be removed through an at least 3 mm incision.
